SEQUENCE LISTING (47 sequences)

SEQ ID NO: 1 - DNA, 477 bp, Bacillus subtilis
  source 1..477: mol_type = genomic DNA
atgactatatgtttcctattattttcttcttattactttagcaatatttcacctcagaatccactgttcaaaaaaaattttttgcaacaattgtctccccaaggctttggcttttatagtaaaagccctacagaagaaaacatttcatttcacacaaaagaaaatttaaagttacctaatgcacttcccaataatttttttgggataaaaagagaaggaagagttcaggcaatagaattaggcaaaattgtagagaatatcgatccaaagaattggaaaacttgtgaaaacaacaactcctgcacaaatttagagaaacaaataaagcctattaaggttataaaaaatgaagattatatacatcttagcaaaggagaatacctaatatatcgccaaaaaccactctcatggtattggatagactttaagcaaactacctcttttgaaagaaaggtgctaaaaataaaaatagtatga

SEQ ID NO: 2 - DNA, 972 bp, Bacillus subtilis
  source 1..972: mol_type = genomic DNA
atgaagatattaaatagtttagaaggttatattgacacctataatccatggaaaaatacatatgcactttttagaagtttacttggtttctcaacattactagtactattattcaatagtactgatattttatttagttatagtgcaaataatgtcacatgtgaaaatgtctatatccctaccgctttttgttttgctaaagaatatagtatcaattttgagattataagatacttaatgatttttatattaaccttagtggttatagggtggagacctagatttaccggtttatttcactggtatatttgctatagtattcaaacttcagctttaactatcgatggtggagagcaaattgcaactgttctttcttttcttatattacctgttacattattagattcaaggcgaaatcattggaatataaagaaaaacaataatgaatctttcacaaagaagacagtattgttttatataatgacaataattaaaattcaagtttttatcatttatttaaacgcagctttagagcgattgaaaaataaagagtgggcagaaggaacagcaatttactatttcttttctgatccggtgtttggattacctgaatatcaacttaacttaatgaatccactacttgaaagcaattttattgttgtcatcacttggttagtaactatttttgagttgttcttagcagcaagcataatttcaaatatcagaataaagagaattgcccttgttttgggaatattatttcatattgggataatattcagcattggtattgtaagttttggcttgatcatgatatcagcattaattatatatctgcatcctgtacaacaaaatatcactatgaattggtgttctcctttatttaaatatatatatgtaaaaggaaagagaaatttcaaaagaataggaggtgaatcagtcaagtttcttacaaaattgtttcatagctaa

SEQ ID NO: 3 - DNA, 612 bp, Bacillus subtilis
  source 1..612: mol_type = genomic DNA
ttgaaaagtaaattacttaggctattgattgtttccatggtaacgatattggttttttcattagtaggactctctaaggagtcaagtacatctgctaaagaaaaccatacattttctggagaagattactttagaggacttttatttggacaaggggaagttggtaaattaatttcaaacgatttggaccctaaactcgtaaaagaggcaaatagtacagaaggtaaaaagttagtaaatgatgtagtcaaatttataaaaaaagatcaaccacaatatatggatgaattgaaacaatcgattgacagcaaagaccctaaaaaactcattgaaaatatgaccaaagcagaccaacttatccaaaaatatgctaagaaaaatgaaaacgtaaaatactcttctaataaagttactccatcttgtgggctttatgccgtctgtgtagcagctggatatttatatgttgtgggcgttaacgcagttgcattacaaacggctgccgcagtaacaactgcagtgtggaaatacgttgccaaatattcctcttcagcttctaataattctgatttagaagcggctgctgcaaaaaccctaaaattgattcatcaataa

SEQ ID NO: 4 - DNA, 273 bp, Bacillus subtilis
  source 1..273: mol_type = genomic DNA
atgaataatgttttcaaagctatatcagatccaacaaggagaaaaattttggatttgttaaaaggaggggatatgactgcgggagatattgctgagcattttaatatcagcaaaccgagtatttcgcaccacttgaatattttaaagcaagcggaagtcataagtgaccatcgcaaaggacaatttatttattactctctaaatacaacagtacttcaagactcaataaattggatgcttaactttataaataaaggggataacgatttatga

SEQ ID NO: 5 - DNA, 624 bp, Bacillus subtilis
  source 1..624: mol_type = genomic DNA
atgaagaaaaatataatttccataattattgtatgtttgagtttcttgacttcaattatattatatcaataccttccagaagaaataccgatacaatggtcaggaaataaaccggctgcaattgtatctaaacctttaacaatatttattatacctgttgttatgcttatttattatcttactttttatatgttaactattaagtctacacaaaaaaataaagcattgcttttcctagcttctaataatatgttaatccttttatatattttacaactctcaactctattgataagtttaggatacgaagtgaatattgatttaataatagggcttggtgttggtatctttttaattattggtggaaattctatgcagctagcagaacaaaaccatcttattggattgaggacaccttggacattaaaagatgagactgtttggaaactgggaaatcgctttgcctctaaagtacttgtagtgtgtggttttataatagctgttctttcattttttacaggagaatatataattcttattatgattgtgctagttcttctagcattagttatttcaacactagcttcatatcattattacaaaaaattaaacggttcacgttga

SEQ ID NO: 6 - DNA, 168 bp, Bacillus subtilis
  source 1..168: mol_type = genomic DNA
atgaaaagaaaccaaaaagaatgggaatctgtgagtaaaaaaggacttatgaagccgggaggtacttcgattgtgaaagctgctggctgcatgggctgttgggcctcgaagagtattgctatgacacgtgtttgtgcacttccgcatcctgctatgagagctatttaa

SEQ ID NO: 7 - DNA, 1233 bp, Bacillus subtilis
  source 1..1233: mol_type = genomic DNA
atgtcatatgatcgtgttaaagattttgatttgccagagttagcggttcatttacagcctcatggtgctgtaatgattgatagaaaaagtatgttttatttcagactcagtggacgtggagcacagttggcgtttctgttatcaaaaaacaaaaatcttcataaaacggcacgtatttgggagattatgaaaaaagaagagatgagtgctgaccaattaaaagaagagcttagtgcccatccatttacggaggcttggacggaagggctgttagatcagcctttacacgtttcgggttcgctagattcatatttacctattagttgtaccttacagttgacaaatgcttgtaatttaagctgttcgttttgctatgccagctcaggtaaaccatatcctgaggaattatctagtgaacaatggatattggttatgcaaaagctagcagcccatggagttgctgatattacgctgaccgggggtgaagcaaagctgatcaaagggtttaaagaattagtcgttgttgcaagttcgttgtttaccaatgtgaatgtatttagtaatggattgaactggcgagatgaagaagttgaattacttagtcacttaggcaatgtttctgtacagattagtattgatggtatggataatacacatgaccaattaagaggcagaaaaggcggctttaaagaaagtatgaataccattaaaaaattatcagaagcaaacattccagtgattgtcgctatgaccattaatgagtccaatgctgatgaagtatcagatgtggtggagcaatgtgccaatgcgggtgcttttatctttcgtgcaggaaaaacgttatctgttggacgtgcgacagaaggctttaaggctttggatattgattttgaagaaatggttcaaatacagcttagggaagcacgtcataaatggggagatcgcctgaacataattgattgggagcacgaagagagttcgttcacaacagatttttgtacaccaggatatcttgcttggtatataagagcagatggatacgtaactccttgccaattagaagatttgccgctagggcatattttagaagatagcatggctgacattggttcacctgctcgtttgcttcagttaaaatgtgaagcaaaaaattgcaaatgtatagggaaaattgagctatctgaaccggacttaccttttcaaaaagaagtcaaggcagggattcaggaatga

SEQ ID NO: 8 - DNA, 1491 bp, Bacillus subtilis
  source 1..1491: mol_type = genomic DNA
atgaatagtctatcattggtgttctggagtattttagcagttgttggattactgttatttattaaattcaaacccccaacaattgcttcactactcttaagcaaagatgaggcaaaagaaataagcattcaatttataaaagagtttgttgggatagatgtagagaactgggatttttattcagtatattggtatgaccacgatacagtaaataaacttcatcacttaggcatacttaagaaaaatagaaaggttttatatgatgttgggttggtcgaatcatggagagtccgtttcgttcaccagaatcaatcatttgtagttggtgtcaatgccaatcgagaaatcacttttttttatgcggatgttccgaaaaaaaccctttcggggaagtttgaacaagtttctccagagacactcaagcagaggttaatggcttcacctgatggactttggtctagagcaaatatgactggtactggtaaaaaagaggaggattttcgcgaggtcagtacttattggtacatagcggaagcgggagatattcggctcaaagtgactgttgaattacagggcggccgaatttcttatattggtactgaacaagaaatactaacagatcaaatgagtaaagtcattcgagatgaacaagtggaatcgacattcggagtatctggtatgctgggttcagctttagcgatgatccttgcgattctcatccttgtatttatggatgtgcaaacaagcataatcttcagtcttgttctgggtttgttgattataatatgccagtcattgacgctgaaagaagatattcaattaacaattgtaaatgcttatgatgcaagaatgagtgtcaaaacggtcagtttattaggtattttgtctacacttcttacaggattattaacaggatttgtagtatttatatgttcattggcaggaaatgcgcttgctggtgattttggatggaaaacgtttgaacaaccaatagttcagattttctatggaataggagcagggctcattagtttaggagtgacttctctgctgtttaacttattggagaaaaagcaatatttacgaatttcacctgagctttctaaccgaactgtctttctatcaggttttacctttaggcaaggattgaatatgagcatacaaagttcaattggagaagaggtcatctatcggctattaatgattccagtcatttggtggatgagtggaaatatcctcatctccattattgtatcttcctttttatgggcggtgatgcaccaagtaactggatatgacccaaggtggatacgttggctgcatctatttatattcggttgctttctgggagttctcttcatcaaatttggttttatttgtgtattagtagctcatttcattcataatttagtactcgtctgtatgccgctgtggcagttcaagcttcagaaacatatgcatcatgatcagccaaagcatacttcactctaa

SEQ ID NO: 9 - DNA, 720 bp, Bacillus subtilis
  source 1..720: mol_type = genomic DNA
atgcaattgatgcaagtacaaaatttaagcaaatgctatcgaaatggtgatggggttgaacatttgtctttctcaattcaacgaggagaaatcgtggcgctattggggccaaatggagctggaaaaacaacgacaattcgatgtttgacaggtctctataagccggataaaggagatatcctcattgagggttctcctccaggagatataaatgttcaaaaaaaagtcgcgcttattcctgatcagccatatttgtatcccgctttaactgctgctgaacatattcagtttagggcaagaggatatcatccaggtaaaaaagatgtaaaagaaagggtttatcatgcattgaaggaagtgcacttggaggagaaagcaaaccaattgtgtggtcagctgtcaagaggccaaaagcagcgggttgtcctggcgggggcgattgttcaagacgctttgctgtatatattagacgaaccgactgtgggccttgatatcccatcaaaacagtggctctctaactggttaaaaactaaaaccgatcaagggtgttcagcatttgtatcaactcatagtctagagtttgttatagagacagcagacagggtgatattaattagagacggcaaactcatgcaggatttgtatgttcctcaattcgaagaacaagcggaatggagaaaagaagtgattcgtcttctaggggaatggtcagatgaataa

SEQ ID NO: 10 - DNA, 1344 bp, Bacillus subtilis
  source 1..1344: mol_type = genomic DNA
atgccttttttgatcatgctcctttttgttggggccatcggatttcaagtaagttttgtttctagatctacgacatgggatatgagtattgctggttgggtacttacaggtgtttttatcctttatacggcatttggacttttttcaaatcgattaccaagtcaaatggcagatattatatggctttatggcactgccacatctttttcaaaagttgtgtatagtgttttatttttcagcgtcacttggaaggcgttgctttggatcatctcagccatattcggtgatgtattaattgtgcttctgtctggtgatcatatcaatttattaggccgatccataatttttgtagggctcttttttatcgctgaagtgtggttgatgtcggtctcttgtgccagaacagtgaagaaaatgaaaagggtatacgttttagtctttcttctaatgttaggcatttactccatatgtctttatcgcttcttctttctccaacattcatctgggatatgggagagcattgcccgttttattagtggcgttggattagtgtttgatacattgtcaccgttgtatgtggttgtatttattgggattatcacagtctcttttatgacaattgcttttacgagccgtcaggtagaaatgaaggaatcgttggtgaaagaagctgaattctgggaggaatttcaagaacgtcaatttggttcaggtcaaattatacagaaaccaaaaacgacttggtggggcttgcaaggtctaaatggcatttggtcttttctgtggttggaactgttgctttttaaaaaatatttattttttcatagcattcatacggtcatgctcagtggcgtcttttatgtcgtcattttcatgtatccagaatggttttatcttctattctttcttatcgtctcggcagtcatgttaagttcctattattcaggaattgtcagacattctcaatcaggcacccttcatttattccccggtgccctttggaaaaaaatcattattctagagctgacgaatacagtctggttgtatattctttactgtgtttctattacttttatggcagttgggaatttagtttattggtatatatatggcttagggatatatatatggttcatgacaataaggctttttgcttttacccatacaaaccgaaacgatattaagctttcattgcctcaatactacaagtcattttttatggcattaggcctgagcggcatttgtttgtatgtcattcatttattgactgctgactggtatacattagtggtggtcgtctgcatagggagcctaagttggtgcctgttttatcgtttcagatag

SEQ ID NO: 11 - DNA, 516 bp, Bacillus subtilis
  source 1..516: mol_type = genomic DNA
atgaattcaaatggtgataaattgtcgttatctgttcagaatttagcgaatacaaatgagatcacaattgttcaagcgataggtgagctaaagaaatcgggaaaagatgcaataccagttttggttgaggccttaaaagaggaaggctctttgtgtaatattgctgcagctgttttaggtgagtttggggaagacgcgagtgaagcagcggaggagctatcatgtttattaaaaagtcatgcggaagatacaagaatggcagcggcgatttcattaatgagaatcgggaagcccagtctgccctttgtcatcaaaatcgctcaagaaagtgaagggcaatcgtgcttttgggcatcttggtgtattgcatggattgatccgtcttgcattgaacctaagatgtataaatgtctaaaatacgagcatgaacatccttcaggaatagttgctccgtttgcggctgaagaggcattaggaaagctgattgcttttcagctgaaagataaggaggactga

SEQ ID NO: 12 - DNA, 426 bp, Bacillus subtilis
  source 1..426: mol_type = genomic DNA
atgaaagatgaacaaatgttgactgaatggccaagtcatttaccatggttgaatcaatcacagaatgattttacatttccaagtgacacatatcttcttctttatttttggtcaatgagctgtccaaactgtcaccaattaacagacaaagtccttcaagatataaaggatatgaatgtgaaagtaatcggagtacatgtcccctatatagaagaagagaaatctatggaggttgtcttgacgtatgctcttgataggggactagctattccgattgtattagaccaaaactatgagatcgtcacaacttgtcacgtacaaggcatccccagcttttgtctattaagtcaatacggtcagatcattactaaaacgatgggagatgttggttgggataagatgttaaaaaagattgcaggcttgtga

SEQ ID NO: 13 - protein, 158 aa, Bacillus subtilis
MTICFLLFSSYYFSNISPQNPLFKKNFLQQLSPQGFGFYSKSPTEENISFHTKENLKLPNALPNNFFGIKREGRVQAIELGKIVENIDPKNWKTCENNNSCTNLEKQIKPIKVIKNEDYIHLSKGEYLIYRQKPLSWYWIDFKQTTSFERKVLKIKIV

SEQ ID NO: 14 - protein, 323 aa, Bacillus subtilis
MKILNSLEGYIDTYNPWKNTYALFRSLLGFSTLLVLLFNSTDILFSYSANNVTCENVYIPTAFCFAKEYSINFEIIRYLMIFILTLVVIGWRPRFTGLFHWYICYSIQTSALTIDGGEQIATVLSFLILPVTLLDSRRNHWNIKKNNNESFTKKTVLFYIMTIIKIQVFIIYLNAALERLKNKEWAEGTAIYYFFSDPVFGLPEYQLNLMNPLLESNFIVVITWLVTIFELFLAASIISNIRIKRIALVLGILFHIGIIFSIGIVSFGLIMISALIIYLHPVQQNITMNWCSPLFKYIYVKGKRNFKRIGGESVKFLTKLFHS

SEQ ID NO: 15 - protein, 203 aa, Bacillus subtilis
MKSKLLRLLIVSMVTILVFSLVGLSKESSTSAKENHTFSGEDYFRGLLFGQGEVGKLISNDLDPKLVKEANSTEGKKLVNDVVKFIKKDQPQYMDELKQSIDSKDPKKLIENMTKADQLIQKYAKKNENVKYSSNKVTPSCGLYAVCVAAGYLYVVGVNAVALQTAAAVTTAVWKYVAKYSSSASNNSDLEAAAAKTLKLIHQ

SEQ ID NO: 16 - protein, 90 aa, Bacillus subtilis
MNNVFKAISDPTRRKILDLLKGGDMTAGDIAEHFNISKPSISHHLNILKQAEVISDHRKGQFIYYSLNTTVLQDSINWMLNFINKGDNDL

SEQ ID NO: 17 - protein, 207 aa, Bacillus subtilis
MKKNIISIIIVCLSFLTSIILYQYLPEEIPIQWSGNKPAAIVSKPLTIFIIPVVMLIYYLTFYMLTIKSTQKNKALLFLASNNMLILLYILQLSTLLISLGYEVNIDLIIGLGVGIFLIIGGNSMQLAEQNHLIGLRTPWTLKDETVWKLGNRFASKVLVVCGFIIAVLSFFTGEYIILIMIVLVLLALVISTLASYHYYKKLNGSR

SEQ ID NO: 18 - protein, 55 aa, Bacillus subtilis
MKRNQKEWESVSKKGLMKPGGTSIVKAAGCMGCWASKSIAMTRVCALPHPAMRAI

SEQ ID NO: 19 - protein, 410 aa, Bacillus subtilis
MSYDRVKDFDLPELAVHLQPHGAVMIDRKSMFYFRLSGRGAQLAFLLSKNKNLHKTARIWEIMKKEEMSADQLKEELSAHPFTEAWTEGLLDQPLHVSGSLDSYLPISCTLQLTNACNLSCSFCYASSGKPYPEELSSEQWILVMQKLAAHGVADITLTGGEAKLIKGFKELVVVASSLFTNVNVFSNGLNWRDEEVELLSHLGNVSVQISIDGMDNTHDQLRGRKGGFKESMNTIKKLSEANIPVIVAMTINESNADEVSDVVEQCANAGAFIFRAGKTLSVGRATEGFKALDIDFEEMVQIQLREARHKWGDRLNIIDWEHEESSFTTDFCTPGYLAWYIRADGYVTPCQLEDLPLGHILEDSMADIGSPARLLQLKCEAKNCKCIGKIELSEPDLPFQKEVKAGIQE

SEQ ID NO: 20 - protein, 496 aa, Bacillus subtilis
MNSLSLVFWSILAVVGLLLFIKFKPPTIASLLLSKDEAKEISIQFIKEFVGIDVENWDFYSVYWYDHDTVNKLHHLGILKKNRKVLYDVGLVESWRVRFVHQNQSFVVGVNANREITFFYADVPKKTLSGKFEQVSPETLKQRLMASPDGLWSRANMTGTGKKEEDFREVSTYWYIAEAGDIRLKVTVELQGGRISYIGTEQEILTDQMSKVIRDEQVESTFGVSGMLGSALAMILAILILVFMDVQTSIIFSLVLGLLIIICQSLTLKEDIQLTIVNAYDARMSVKTVSLLGILSTLLTGLLTGFVVFICSLAGNALAGDFGWKTFEQPIVQIFYGIGAGLISLGVTSLLFNLLEKKQYLRISPELSNRTVFLSGFTFRQGLNMSIQSSIGEEVIYRLLMIPVIWWMSGNILISIIVSSFLWAVMHQVTGYDPRWIRWLHLFIFGCFLGVLFIKFGFICVLVAHFIHNLVLVCMPLWQFKLQKHMHHDQPKHTSL

SEQ ID NO: 21 - protein, 239 aa, Bacillus subtilis
MQLMQVQNLSKCYRNGDGVEHLSFSIQRGEIVALLGPNGAGKTTTIRCLTGLYKPDKGDILIEGSPPGDINVQKKVALIPDQPYLYPALTAAEHIQFRARGYHPGKKDVKERVYHALKEVHLEEKANQLCGQLSRGQKQRVVLAGAIVQDALLYILDEPTVGLDIPSKQWLSNWLKTKTDQGCSAFVSTHSLEFVIETADRVILIRDGKLMQDLYVPQFEEQAEWRKEVIRLLGEWSDE

SEQ ID NO: 22 - protein, 447 aa, Bacillus subtilis
MPFLIMLLFVGAIGFQVSFVSRSTTWDMSIAGWVLTGVFILYTAFGLFSNRLPSQMADIIWLYGTATSFSKVVYSVLFFSVTWKALLWIISAIFGDVLIVLLSGDHINLLGRSIIFVGLFFIAEVWLMSVSCARTVKKMKRVYVLVFLLMLGIYSICLYRFFFLQHSSGIWESIARFISGVGLVFDTLSPLYVVVFIGIITVSFMTIAFTSRQVEMKESLVKEAEFWEEFQERQFGSGQIIQKPKTTWWGLQGLNGIWSFLWLELLLFKKYLFFHSIHTVMLSGVFYVVIFMYPEWFYLLFFLIVSAVMLSSYYSGIVRHSQSGTLHLFPGALWKKIIILELTNTVWLYILYCVSITFMAVGNLVYWYIYGLGIYIWFMTIRLFAFTHTNRNDIKLSLPQYYKSFFMALGLSGICLYVIHLLTADWYTLVVVVCIGSLSWCLFYRFR

SEQ ID NO: 23 - protein, 171 aa, Bacillus subtilis
MNSNGDKLSLSVQNLANTNEITIVQAIGELKKSGKDAIPVLVEALKEEGSLCNIAAAVLGEFGEDASEAAEELSCLLKSHAEDTRMAAAISLMRIGKPSLPFVIKIAQESEGQSCFWASWCIAWIDPSCIEPKMYKCLKYEHEHPSGIVAPFAAEEALGKLIAFQLKDKED

SEQ ID NO: 24 - protein, 141 aa, Bacillus subtilis
MKDEQMLTEWPSHLPWLNQSQNDFTFPSDTYLLLYFWSMSCPNCHQLTDKVLQDIKDMNVKVIGVHVPYIEEEKSMEVVLTYALDRGLAIPIVLDQNYEIVTTCHVQGIPSFCLLSQYGQIITKTMGDVGWDKMLKKIAGL

SEQ ID NO: 25 - DNA, 603 bp, synthetic construct
  source 1..603: mol_type = other DNA
atgagacctaagcatccgattaaacatcaaggcctgcctcaagaagttctgaatgaaaatctgctgagattctttgtggcaccgtttcctgaggtgttcggcaaagaaaaagttaatgaactgtcaaaagatattggctcagaatcaacagaagatcaagcaatggaagatattaaacaaatggaagctgaaagcattagctcatcagaagagattgttccgaatagcgtggaacaaaaacatattcaaaaggaggatgttccgagcgaaagatatctgggctatcttgaacaacttctgagacttaaaaagtataaggttccgcaacttgaaattgttccgaatagcgcagaagagagacttcatagcatgaaagaaggcattcatgcgcaacagaaagaaccgatgattggagttaatcaagaacttgcgtatttttatccggaactgtttagacaattctatcaactggatgcgtatccgtcaggcgcttggtattacgttccgctgggaacacaatatacagatgctccgtcttttagcgatattccgaatccgattggctctgaaaactctgaaaagacaacgatgccgctgtggtaa

SEQ ID NO: 26 - protein, 200 aa, Bos taurus
MRPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 27 - DNA, 2099 bp, synthetic construct
  source 1..2099: mol_type = other DNA
atgactatatgtttcctattattttcttcttattactttagcaatatttcacctcagaatccactgttcaaaaaaaattttttgcaacaattgtctccccaaggctttggcttttatagtaaaagccctacagaagaaaacatttcatttcacacaaaagaaaatttaaagttacctaatgcacttcccaataatttttttgggataaaaagagaaggaagagttcaggcaatagaattaggcaaaattgtagagaatatcgatccaaagaattggaaaacttgtgaaaacaacaactcctgcacaaatttagagaaacaaataaagcctattaaggttataaaaaatgaagattatatacatcttagcaaaggagaatacctaatatatcgccaaaaaccactctcatggtattggatagactttaagcaaactacctcttttgaaagaaaggtgctaaaaataaaaatagtatgaagatattaaatagtttagaaggttatattgacacctataatccatggaaaaatacatatgcactttttagaagtttacttggtttctcaacattactagtactattattcaatagtactgatattttatttagttatagtgcaaataatgtcacatgtgaaaatgtctatatccctaccgctttttgttttgctaaagaatatagtatcaattttgagattataagatacttaatgatttttatattaaccttagtggttatagggtggagacctagatttaccggtttatttcactggtatatttgctatagtattcaaacttcagctttaactatcgatggtggagagcaaattgcaactgttctttcttttcttatattacctgttacattattagattcaaggcgaaatcattggaatataaagaaaaacaataatgaatctttcacaaagaagacagtattgttttatataatgacaataattaaaattcaagtttttatcatttatttaaacgcagctttagagcgattgaaaaataaagagtgggcagaaggaacagcaatttactatttcttttctgatccggtgtttggattacctgaatatcaacttaacttaatgaatccactacttgaaagcaattttattgttgtcatcacttggttagtaactatttttgagttgttcttagcagcaagcataatttcaaatatcagaataaagagaattgcccttgttttgggaatattatttcatattgggataatattcagcattggtattgtaagttttggcttgatcatgatatcagcattaattatatatctgcatcctgtacaacaaaatatcactatgaattggtgttctcctttatttaaatatatatatgtaaaaggaaagagaaatttcaaaagaataggaggtgaatcagtcaagtttcttacaaaattgtttcatagctaacatttagataatggagaaataacttaatggaggtataataatttgaaaagtaaattacttaggctattgattgtttccatggtaacgatattggttttttcattagtaggactctctaaggagtcaagtacatctgctaaagaaaaccatacattttctggagaagattactttagaggacttttatttggacaaggggaagttggtaaattaatttcaaacgatttggaccctaaactcgtaaaagaggcaaatagtacagaaggtaaaaagttagtaaatgatgtagtcaaatttataaaaaaagatcaaccacaatatatggatgaattgaaacaatcgattgacagcaaagaccctaaaaaactcattgaaaatatgaccaaagcagaccaacttatccaaaaatatgctaagaaaaatgaaaacgtaaaatactcttctaataaagttactccatcttgtgggctttatgccgtctgtgtagcagctggatatttatatgttgtgggcgttaacgcagttgcattacaaacggctgccgcagtaacaactgcagtgtggaaatacgttgccaaatattcctcttcagcttctaataattctgatttagaagcggctgctgcaaaaaccctaaaattgattcatcaataa

SEQ ID NO: 28 - DNA, 2954 bp, synthetic construct
  source 1..2954: mol_type = other DNA
atgaaaagaaaccaaaaagaatgggaatctgtgagtaaaaaaggacttatgaagccgggaggtacttcgattgtgaaagctgctggctgcatgggctgttgggcctcgaagagtattgctatgacacgtgtttgtgcacttccgcatcctgctatgagagctatttaacatttgagaatagggagttgagcgtatttgcttatactccttattttctcttaagggggattttatatgtcatatgatcgtgttaaagattttgatttgccagagttagcggttcatttacagcctcatggtgctgtaatgattgatagaaaaagtatgttttatttcagactcagtggacgtggagcacagttggcgtttctgttatcaaaaaacaaaaatcttcataaaacggcacgtatttgggagattatgaaaaaagaagagatgagtgctgaccaattaaaagaagagcttagtgcccatccatttacggaggcttggacggaagggctgttagatcagcctttacacgtttcgggttcgctagattcatatttacctattagttgtaccttacagttgacaaatgcttgtaatttaagctgttcgttttgctatgccagctcaggtaaaccatatcctgaggaattatctagtgaacaatggatattggttatgcaaaagctagcagcccatggagttgctgatattacgctgaccgggggtgaagcaaagctgatcaaagggtttaaagaattagtcgttgttgcaagttcgttgtttaccaatgtgaatgtatttagtaatggattgaactggcgagatgaagaagttgaattacttagtcacttaggcaatgtttctgtacagattagtattgatggtatggataatacacatgaccaattaagaggcagaaaaggcggctttaaagaaagtatgaataccattaaaaaattatcagaagcaaacattccagtgattgtcgctatgaccattaatgagtccaatgctgatgaagtatcagatgtggtggagcaatgtgccaatgcgggtgcttttatctttcgtgcaggaaaaacgttatctgttggacgtgcgacagaaggctttaaggctttggatattgattttgaagaaatggttcaaatacagcttagggaagcacgtcataaatggggagatcgcctgaacataattgattgggagcacgaagagagttcgttcacaacagatttttgtacaccaggatatcttgcttggtatataagagcagatggatacgtaactccttgccaattagaagatttgccgctagggcatattttagaagatagcatggctgacattggttcacctgctcgtttgcttcagttaaaatgtgaagcaaaaaattgcaaatgtatagggaaaattgagctatctgaaccggacttaccttttcaaaaagaagtcaaggcagggattcaggaatgaatagtctatcattggtgttctggagtattttagcagttgttggattactgttatttattaaattcaaacccccaacaattgcttcactactcttaagcaaagatgaggcaaaagaaataagcattcaatttataaaagagtttgttgggatagatgtagagaactgggatttttattcagtatattggtatgaccacgatacagtaaataaacttcatcacttaggcatacttaagaaaaatagaaaggttttatatgatgttgggttggtcgaatcatggagagtccgtttcgttcaccagaatcaatcatttgtagttggtgtcaatgccaatcgagaaatcacttttttttatgcggatgttccgaaaaaaaccctttcggggaagtttgaacaagtttctccagagacactcaagcagaggttaatggcttcacctgatggactttggtctagagcaaatatgactggtactggtaaaaaagaggaggattttcgcgaggtcagtacttattggtacatagcggaagcgggagatattcggctcaaagtgactgttgaattacagggcggccgaatttcttatattggtactgaacaagaaatactaacagatcaaatgagtaaagtcattcgagatgaacaagtggaatcgacattcggagtatctggtatgctgggttcagctttagcgatgatccttgcgattctcatccttgtatttatggatgtgcaaacaagcataatcttcagtcttgttctgggtttgttgattataatatgccagtcattgacgctgaaagaagatattcaattaacaattgtaaatgcttatgatgcaagaatgagtgtcaaaacggtcagtttattaggtattttgtctacacttcttacaggattattaacaggatttgtagtatttatatgttcattggcaggaaatgcgcttgctggtgattttggatggaaaacgtttgaacaaccaatagttcagattttctatggaataggagcagggctcattagtttaggagtgacttctctgctgtttaacttattggagaaaaagcaatatttacgaatttcacctgagctttctaaccgaactgtctttctatcaggttttacctttaggcaaggattgaatatgagcatacaaagttcaattggagaagaggtcatctatcggctattaatgattccagtcatttggtggatgagtggaaatatcctcatctccattattgtatcttcctttttatgggcggtgatgcaccaagtaactggatatgacccaaggtggatacgttggctgcatctatttatattcggttgctttctgggagttctcttcatcaaatttggttttatttgtgtattagtagctcatttcattcataatttagtactcgtctgtatgccgctgtggcagttcaagcttcagaaacatatgcatcatgatcagccaaagcatacttcactctaa

SEQ ID NO: 29 - DNA, 6092 bp, synthetic construct
  source 1..6092: mol_type = other DNA
atgaaaagaaaccaaaaagaatgggaatctgtgagtaaaaaaggacttatgaagccgggaggtacttcgattgtgaaagctgctggctgcatgggctgttgggcctcgaagagtattgctatgacacgtgtttgtgcacttccgcatcctgctatgagagctatttaacatttgagaatagggagttgagcgtatttgcttatactccttattttctcttaagggggattttatatgtcatatgatcgtgttaaagattttgatttgccagagttagcggttcatttacagcctcatggtgctgtaatgattgatagaaaaagtatgttttatttcagactcagtggacgtggagcacagttggcgtttctgttatcaaaaaacaaaaatcttcataaaacggcacgtatttgggagattatgaaaaaagaagagatgagtgctgaccaattaaaagaagagcttagtgcccatccatttacggaggcttggacggaagggctgttagatcagcctttacacgtttcgggttcgctagattcatatttacctattagttgtaccttacagttgacaaatgcttgtaatttaagctgttcgttttgctatgccagctcaggtaaaccatatcctgaggaattatctagtgaacaatggatattggttatgcaaaagctagcagcccatggagttgctgatattacgctgaccgggggtgaagcaaagctgatcaaagggtttaaagaattagtcgttgttgcaagttcgttgtttaccaatgtgaatgtatttagtaatggattgaactggcgagatgaagaagttgaattacttagtcacttaggcaatgtttctgtacagattagtattgatggtatggataatacacatgaccaattaagaggcagaaaaggcggctttaaagaaagtatgaataccattaaaaaattatcagaagcaaacattccagtgattgtcgctatgaccattaatgagtccaatgctgatgaagtatcagatgtggtggagcaatgtgccaatgcgggtgcttttatctttcgtgcaggaaaaacgttatctgttggacgtgcgacagaaggctttaaggctttggatattgattttgaagaaatggttcaaatacagcttagggaagcacgtcataaatggggagatcgcctgaacataattgattgggagcacgaagagagttcgttcacaacagatttttgtacaccaggatatcttgcttggtatataagagcagatggatacgtaactccttgccaattagaagatttgccgctagggcatattttagaagatagcatggctgacattggttcacctgctcgtttgcttcagttaaaatgtgaagcaaaaaattgcaaatgtatagggaaaattgagctatctgaaccggacttaccttttcaaaaagaagtcaaggcagggattcaggaatgaatagtctatcattggtgttctggagtattttagcagttgttggattactgttatttattaaattcaaacccccaacaattgcttcactactcttaagcaaagatgaggcaaaagaaataagcattcaatttataaaagagtttgttgggatagatgtagagaactgggatttttattcagtatattggtatgaccacgatacagtaaataaacttcatcacttaggcatacttaagaaaaatagaaaggttttatatgatgttgggttggtcgaatcatggagagtccgtttcgttcaccagaatcaatcatttgtagttggtgtcaatgccaatcgagaaatcacttttttttatgcggatgttccgaaaaaaaccctttcggggaagtttgaacaagtttctccagagacactcaagcagaggttaatggcttcacctgatggactttggtctagagcaaatatgactggtactggtaaaaaagaggaggattttcgcgaggtcagtacttattggtacatagcggaagcgggagatattcggctcaaagtgactgttgaattacagggcggccgaatttcttatattggtactgaacaagaaatactaacagatcaaatgagtaaagtcattcgagatgaacaagtggaatcgacattcggagtatctggtatgctgggttcagctttagcgatgatccttgcgattctcatccttgtatttatggatgtgcaaacaagcataatcttcagtcttgttctgggtttgttgattataatatgccagtcattgacgctgaaagaagatattcaattaacaattgtaaatgcttatgatgcaagaatgagtgtcaaaacggtcagtttattaggtattttgtctacacttcttacaggattattaacaggatttgtagtatttatatgttcattggcaggaaatgcgcttgctggtgattttggatggaaaacgtttgaacaaccaatagttcagattttctatggaataggagcagggctcattagtttaggagtgacttctctgctgtttaacttattggagaaaaagcaatatttacgaatttcacctgagctttctaaccgaactgtctttctatcaggttttacctttaggcaaggattgaatatgagcatacaaagttcaattggagaagaggtcatctatcggctattaatgattccagtcatttggtggatgagtggaaatatcctcatctccattattgtatcttcctttttatgggcggtgatgcaccaagtaactggatatgacccaaggtggatacgttggctgcatctatttatattcggttgctttctgggagttctcttcatcaaatttggttttatttgtgtattagtagctcatttcattcataatttagtactcgtctgtatgccgctgtggcagttcaagcttcagaaacatatgcatcatgatcagccaaagcatacttcactctaatgattaggagaagatttgatgcaattgatgcaagtacaaaatttaagcaaatgctatcgaaatggtgatggggttgaacatttgtctttctcaattcaacgaggagaaatcgtggcgctattggggccaaatggagctggaaaaacaacgacaattcgatgtttgacaggtctctataagccggataaaggagatatcctcattgagggttctcctccaggagatataaatgttcaaaaaaaagtcgcgcttattcctgatcagccatatttgtatcccgctttaactgctgctgaacatattcagtttagggcaagaggatatcatccaggtaaaaaagatgtaaaagaaagggtttatcatgcattgaaggaagtgcacttggaggagaaagcaaaccaattgtgtggtcagctgtcaagaggccaaaagcagcgggttgtcctggcgggggcgattgttcaagacgctttgctgtatatattagacgaaccgactgtgggccttgatatcccatcaaaacagtggctctctaactggttaaaaactaaaaccgatcaagggtgttcagcatttgtatcaactcatagtctagagtttgttatagagacagcagacagggtgatattaattagagacggcaaactcatgcaggatttgtatgttcctcaattcgaagaacaagcggaatggagaaaagaagtgattcgtcttctaggggaatggtcagatgaataatatcatttcttttttatggcttcagtcaaaaaggcgttttgtaagtcaaggtcaggaaaaaaaaatgccttttttgatcatgctcctttttgttggggccatcggatttcaagtaagttttgtttctagatctacgacatgggatatgagtattgctggttgggtacttacaggtgtttttatcctttatacggcatttggacttttttcaaatcgattaccaagtcaaatggcagatattatatggctttatggcactgccacatctttttcaaaagttgtgtatagtgttttatttttcagcgtcacttggaaggcgttgctttggatcatctcagccatattcggtgatgtattaattgtgcttctgtctggtgatcatatcaatttattaggccgatccataatttttgtagggctcttttttatcgctgaagtgtggttgatgtcggtctcttgtgccagaacagtgaagaaaatgaaaagggtatacgttttagtctttcttctaatgttaggcatttactccatatgtctttatcgcttcttctttctccaacattcatctgggatatgggagagcattgcccgttttattagtggcgttggattagtgtttgatacattgtcaccgttgtatgtggttgtatttattgggattatcacagtctcttttatgacaattgcttttacgagccgtcaggtagaaatgaaggaatcgttggtgaaagaagctgaattctgggaggaatttcaagaacgtcaatttggttcaggtcaaattatacagaaaccaaaaacgacttggtggggcttgcaaggtctaaatggcatttggtcttttctgtggttggaactgttgctttttaaaaaatatttattttttcatagcattcatacggtcatgctcagtggcgtcttttatgtcgtcattttcatgtatccagaatggttttatcttctattctttcttatcgtctcggcagtcatgttaagttcctattattcaggaattgtcagacattctcaatcaggcacccttcatttattccccggtgccctttggaaaaaaatcattattctagagctgacgaatacagtctggttgtatattctttactgtgtttctattacttttatggcagttgggaatttagtttattggtatatatatggcttagggatatatatatggttcatgacaataaggctttttgcttttacccatacaaaccgaaacgatattaagctttcattgcctcaatactacaagtcattttttatggcattaggcctgagcggcatttgtttgtatgtcattcatttattgactgctgactggtatacattagtggtggtcgtctgcatagggagcctaagttggtgcctgttttatcgtttcagatagtggttatgtagagttattgttttaaaacgaggagaggggctcttttatgaattcaaatggtgataaattgtcgttatctgttcagaatttagcgaatacaaatgagatcacaattgttcaagcgataggtgagctaaagaaatcgggaaaagatgcaataccagttttggttgaggccttaaaagaggaaggctctttgtgtaatattgctgcagctgttttaggtgagtttggggaagacgcgagtgaagcagcggaggagctatcatgtttattaaaaagtcatgcggaagatacaagaatggcagcggcgatttcattaatgagaatcgggaagcccagtctgccctttgtcatcaaaatcgctcaagaaagtgaagggcaatcgtgcttttgggcatcttggtgtattgcatggattgatccgtcttgcattgaacctaagatgtataaatgtctaaaatacgagcatgaacatccttcaggaatagttgctccgtttgcggctgaagaggcattaggaaagctgattgcttttcagctgaaagataaggaggactgaaaggatgaaagatgaacaaatgttgactgaatggccaagtcatttaccatggttgaatcaatcacagaatgattttacatttccaagtgacacatatcttcttctttatttttggtcaatgagctgtccaaactgtcaccaattaacagacaaagtccttcaagatataaaggatatgaatgtgaaagtaatcggagtacatgtcccctatatagaagaagagaaatctatggaggttgtcttgacgtatgctcttgataggggactagctattccgattgtattagaccaaaactatgagatcgtcacaacttgtcacgtacaaggcatccccagcttttgtctattaagtcaatacggtcagatcattactaaaacgatgggagatgttggttgggataagatgttaaaaaagattgcaggcttgtga

SEQ ID NO: 30 - DNA, 6456 bp, synthetic construct
  source 1..6456: mol_type = other DNA
ttcttccctccgaacgaaccgcagttttgtccatatatgcctttttataacctatgagacaagttccttgaaaagacgaagaaaacatgttttactttgtaacaaatcaaaaatttttgtgcataagacttgaaagtcaaagatagtcagagtatactattaatcaaagttggtcaaacaaaccggccttttttaaaaatcaattggtcaaagatagtcaaatattcagtctgctttgagcatattggtttgaatgccgttaagtttgccgtatactaatagtcaaagaaggtcaaacccaatagacctttttattcactttcattggtcaaagatgatcaaattattaaggaggttttggcaaatgaaaagaaaccaaaaagaatgggaatctgtgagtaaaaaaggacttatgaagccgggaggtacttcgattgtgaaagctgctggctgcatgggctgttgggcctcgaagagtattgctatgacacgtgtttgtgcacttccgcatcctgctatgagagctatttaacatttgagaatagggagttgagcgtatttgcttatactccttattttctcttaagggggattttatatgtcatatgatcgtgttaaagattttgatttgccagagttagcggttcatttacagcctcatggtgctgtaatgattgatagaaaaagtatgttttatttcagactcagtggacgtggagcacagttggcgtttctgttatcaaaaaacaaaaatcttcataaaacggcacgtatttgggagattatgaaaaaagaagagatgagtgctgaccaattaaaagaagagcttagtgcccatccatttacggaggcttggacggaagggctgttagatcagcctttacacgtttcgggttcgctagattcatatttacctattagttgtaccttacagttgacaaatgcttgtaatttaagctgttcgttttgctatgccagctcaggtaaaccatatcctgaggaattatctagtgaacaatggatattggttatgcaaaagctagcagcccatggagttgctgatattacgctgaccgggggtgaagcaaagctgatcaaagggtttaaagaattagtcgttgttgcaagttcgttgtttaccaatgtgaatgtatttagtaatggattgaactggcgagatgaagaagttgaattacttagtcacttaggcaatgtttctgtacagattagtattgatggtatggataatacacatgaccaattaagaggcagaaaaggcggctttaaagaaagtatgaataccattaaaaaattatcagaagcaaacattccagtgattgtcgctatgaccattaatgagtccaatgctgatgaagtatcagatgtggtggagcaatgtgccaatgcgggtgcttttatctttcgtgcaggaaaaacgttatctgttggacgtgcgacagaaggctttaaggctttggatattgattttgaagaaatggttcaaatacagcttagggaagcacgtcataaatggggagatcgcctgaacataattgattgggagcacgaagagagttcgttcacaacagatttttgtacaccaggatatcttgcttggtatataagagcagatggatacgtaactccttgccaattagaagatttgccgctagggcatattttagaagatagcatggctgacattggttcacctgctcgtttgcttcagttaaaatgtgaagcaaaaaattgcaaatgtatagggaaaattgagctatctgaaccggacttaccttttcaaaaagaagtcaaggcagggattcaggaatgaatagtctatcattggtgttctggagtattttagcagttgttggattactgttatttattaaattcaaacccccaacaattgcttcactactcttaagcaaagatgaggcaaaagaaataagcattcaatttataaaagagtttgttgggatagatgtagagaactgggatttttattcagtatattggtatgaccacgatacagtaaataaacttcatcacttaggcatacttaagaaaaatagaaaggttttatatgatgttgggttggtcgaatcatggagagtccgtttcgttcaccagaatcaatcatttgtagttggtgtcaatgccaatcgagaaatcacttttttttatgcggatgttccgaaaaaaaccctttcggggaagtttgaacaagtttctccagagacactcaagcagaggttaatggcttcacctgatggactttggtctagagcaaatatgactggtactggtaaaaaagaggaggattttcgcgaggtcagtacttattggtacatagcggaagcgggagatattcggctcaaagtgactgttgaattacagggcggccgaatttcttatattggtactgaacaagaaatactaacagatcaaatgagtaaagtcattcgagatgaacaagtggaatcgacattcggagtatctggtatgctgggttcagctttagcgatgatccttgcgattctcatccttgtatttatggatgtgcaaacaagcataatcttcagtcttgttctgggtttgttgattataatatgccagtcattgacgctgaaagaagatattcaattaacaattgtaaatgcttatgatgcaagaatgagtgtcaaaacggtcagtttattaggtattttgtctacacttcttacaggattattaacaggatttgtagtatttatatgttcattggcaggaaatgcgcttgctggtgattttggatggaaaacgtttgaacaaccaatagttcagattttctatggaataggagcagggctcattagtttaggagtgacttctctgctgtttaacttattggagaaaaagcaatatttacgaatttcacctgagctttctaaccgaactgtctttctatcaggttttacctttaggcaaggattgaatatgagcatacaaagttcaattggagaagaggtcatctatcggctattaatgattccagtcatttggtggatgagtggaaatatcctcatctccattattgtatcttcctttttatgggcggtgatgcaccaagtaactggatatgacccaaggtggatacgttggctgcatctatttatattcggttgctttctgggagttctcttcatcaaatttggttttatttgtgtattagtagctcatttcattcataatttagtactcgtctgtatgccgctgtggcagttcaagcttcagaaacatatgcatcatgatcagccaaagcatacttcactctaatgattaggagaagatttgatgcaattgatgcaagtacaaaatttaagcaaatgctatcgaaatggtgatggggttgaacatttgtctttctcaattcaacgaggagaaatcgtggcgctattggggccaaatggagctggaaaaacaacgacaattcgatgtttgacaggtctctataagccggataaaggagatatcctcattgagggttctcctccaggagatataaatgttcaaaaaaaagtcgcgcttattcctgatcagccatatttgtatcccgctttaactgctgctgaacatattcagtttagggcaagaggatatcatccaggtaaaaaagatgtaaaagaaagggtttatcatgcattgaaggaagtgcacttggaggagaaagcaaaccaattgtgtggtcagctgtcaagaggccaaaagcagcgggttgtcctggcgggggcgattgttcaagacgctttgctgtatatattagacgaaccgactgtgggccttgatatcccatcaaaacagtggctctctaactggttaaaaactaaaaccgatcaagggtgttcagcatttgtatcaactcatagtctagagtttgttatagagacagcagacagggtgatattaattagagacggcaaactcatgcaggatttgtatgttcctcaattcgaagaacaagcggaatggagaaaagaagtgattcgtcttctaggggaatggtcagatgaataatatcatttcttttttatggcttcagtcaaaaaggcgttttgtaagtcaaggtcaggaaaaaaaaatgccttttttgatcatgctcctttttgttggggccatcggatttcaagtaagttttgtttctagatctacgacatgggatatgagtattgctggttgggtacttacaggtgtttttatcctttatacggcatttggacttttttcaaatcgattaccaagtcaaatggcagatattatatggctttatggcactgccacatctttttcaaaagttgtgtatagtgttttatttttcagcgtcacttggaaggcgttgctttggatcatctcagccatattcggtgatgtattaattgtgcttctgtctggtgatcatatcaatttattaggccgatccataatttttgtagggctcttttttatcgctgaagtgtggttgatgtcggtctcttgtgccagaacagtgaagaaaatgaaaagggtatacgttttagtctttcttctaatgttaggcatttactccatatgtctttatcgcttcttctttctccaacattcatctgggatatgggagagcattgcccgttttattagtggcgttggattagtgtttgatacattgtcaccgttgtatgtggttgtatttattgggattatcacagtctcttttatgacaattgcttttacgagccgtcaggtagaaatgaaggaatcgttggtgaaagaagctgaattctgggaggaatttcaagaacgtcaatttggttcaggtcaaattatacagaaaccaaaaacgacttggtggggcttgcaaggtctaaatggcatttggtcttttctgtggttggaactgttgctttttaaaaaatatttattttttcatagcattcatacggtcatgctcagtggcgtcttttatgtcgtcattttcatgtatccagaatggttttatcttctattctttcttatcgtctcggcagtcatgttaagttcctattattcaggaattgtcagacattctcaatcaggcacccttcatttattccccggtgccctttggaaaaaaatcattattctagagctgacgaatacagtctggttgtatattctttactgtgtttctattacttttatggcagttgggaatttagtttattggtatatatatggcttagggatatatatatggttcatgacaataaggctttttgcttttacccatacaaaccgaaacgatattaagctttcattgcctcaatactacaagtcattttttatggcattaggcctgagcggcatttgtttgtatgtcattcatttattgactgctgactggtatacattagtggtggtcgtctgcatagggagcctaagttggtgcctgttttatcgtttcagatagtggttatgtagagttattgttttaaaacgaggagaggggctcttttatgaattcaaatggtgataaattgtcgttatctgttcagaatttagcgaatacaaatgagatcacaattgttcaagcgataggtgagctaaagaaatcgggaaaagatgcaataccagttttggttgaggccttaaaagaggaaggctctttgtgtaatattgctgcagctgttttaggtgagtttggggaagacgcgagtgaagcagcggaggagctatcatgtttattaaaaagtcatgcggaagatacaagaatggcagcggcgatttcattaatgagaatcgggaagcccagtctgccctttgtcatcaaaatcgctcaagaaagtgaagggcaatcgtgcttttgggcatcttggtgtattgcatggattgatccgtcttgcattgaacctaagatgtataaatgtctaaaatacgagcatgaacatccttcaggaatagttgctccgtttgcggctgaagaggcattaggaaagctgattgcttttcagctgaaagataaggaggactgaaaggatgaaagatgaacaaatgttgactgaatggccaagtcatttaccatggttgaatcaatcacagaatgattttacatttccaagtgacacatatcttcttctttatttttggtcaatgagctgtccaaactgtcaccaattaacagacaaagtccttcaagatataaaggatatgaatgtgaaagtaatcggagtacatgtcccctatatagaagaagagaaatctatggaggttgtcttgacgtatgctcttgataggggactagctattccgattgtattagaccaaaactatgagatcgtcacaacttgtcacgtacaaggcatccccagcttttgtctattaagtcaatacggtcagatcattactaaaacgatgggagatgttggttgggataagatgttaaaaaagattgcaggcttgtga

SEQ ID NO: 31 - DNA, 2463 bp, synthetic construct
  source 1..2463: mol_type = other DNA
ttcttccctccgaacgaaccgcagttttgtccatatatgcctttttataacctatgagacaagttccttgaaaagacgaagaaaacatgttttactttgtaacaaatcaaaaatttttgtgcataagacttgaaagtcaaagatagtcagagtatactattaatcaaagttggtcaaacaaaccggccttttttaaaaatcaattggtcaaagatagtcaaatattcagtctgctttgagcatattggtttgaatgccgttaagtttgccgtatactaatagtcaaagaaggtcaaacccaatagacctttttattcactttcattggtcaaagatgatcaaattattaaggaggttttggcaaatgactatatgtttcctattattttcttcttattactttagcaatatttcacctcagaatccactgttcaaaaaaaattttttgcaacaattgtctccccaaggctttggcttttatagtaaaagccctacagaagaaaacatttcatttcacacaaaagaaaatttaaagttacctaatgcacttcccaataatttttttgggataaaaagagaaggaagagttcaggcaatagaattaggcaaaattgtagagaatatcgatccaaagaattggaaaacttgtgaaaacaacaactcctgcacaaatttagagaaacaaataaagcctattaaggttataaaaaatgaagattatatacatcttagcaaaggagaatacctaatatatcgccaaaaaccactctcatggtattggatagactttaagcaaactacctcttttgaaagaaaggtgctaaaaataaaaatagtatgaagatattaaatagtttagaaggttatattgacacctataatccatggaaaaatacatatgcactttttagaagtttacttggtttctcaacattactagtactattattcaatagtactgatattttatttagttatagtgcaaataatgtcacatgtgaaaatgtctatatccctaccgctttttgttttgctaaagaatatagtatcaattttgagattataagatacttaatgatttttatattaaccttagtggttatagggtggagacctagatttaccggtttatttcactggtatatttgctatagtattcaaacttcagctttaactatcgatggtggagagcaaattgcaactgttctttcttttcttatattacctgttacattattagattcaaggcgaaatcattggaatataaagaaaaacaataatgaatctttcacaaagaagacagtattgttttatataatgacaataattaaaattcaagtttttatcatttatttaaacgcagctttagagcgattgaaaaataaagagtgggcagaaggaacagcaatttactatttcttttctgatccggtgtttggattacctgaatatcaacttaacttaatgaatccactacttgaaagcaattttattgttgtcatcacttggttagtaactatttttgagttgttcttagcagcaagcataatttcaaatatcagaataaagagaattgcccttgttttgggaatattatttcatattgggataatattcagcattggtattgtaagttttggcttgatcatgatatcagcattaattatatatctgcatcctgtacaacaaaatatcactatgaattggtgttctcctttatttaaatatatatatgtaaaaggaaagagaaatttcaaaagaataggaggtgaatcagtcaagtttcttacaaaattgtttcatagctaacatttagataatggagaaataacttaatggaggtataataatttgaaaagtaaattacttaggctattgattgtttccatggtaacgatattggttttttcattagtaggactctctaaggagtcaagtacatctgctaaagaaaaccatacattttctggagaagattactttagaggacttttatttggacaaggggaagttggtaaattaatttcaaacgatttggaccctaaactcgtaaaagaggcaaatagtacagaaggtaaaaagttagtaaatgatgtagtcaaatttataaaaaaagatcaaccacaatatatggatgaattgaaacaatcgattgacagcaaagaccctaaaaaactcattgaaaatatgaccaaagcagaccaacttatccaaaaatatgctaagaaaaatgaaaacgtaaaatactcttctaataaagttactccatcttgtgggctttatgccgtctgtgtagcagctggatatttatatgttgtgggcgttaacgcagttgcattacaaacggctgccgcagtaacaactgcagtgtggaaatacgttgccaaatattcctcttcagcttctaataattctgatttagaagcggctgctgcaaaaaccctaaaattgattcatcaataa

SEQ ID NO: 32 - DNA, 309 bp, Bacillus subtilis
  source 1..309: mol_type = genomic DNA
atgaaaaaatttattgctttactgttctttatattgcttctttcgggttgcggggttaatagtcaaaagagtcaaggtgaagatgtatcgccagacagtaacattgaaacaaaagaaggtacttatgtagggttagctgatactcatacaatagaagtaacagtagataatgagccggttagtcttgatatcactgaagaatcgacaagtgatcttgataagtttaacagtggagataaggtcacgattacatatgaaaaaaatgatgagggtcagcttctgttaaaagatattgaacgtgccaactaa

SEQ ID NO: 33 - DNA, 2118 bp, Bacillus subtilis
  source 1..2118: mol_type = genomic DNA
tcaataacttgtcagagttgtacctggatagtaaaatttcaaaattgaactatatgaatctccagcttctgctcttgcttttgctccatattgactcattccgatcccgtgaccatatcctttaccgctgattgtgtactttgaagtatctttttttacagtcacataagtacttttaaatacagtcgctccaatcattgttcttaactcacttgttggtacactaatggttgtaattttgctcaagttataggagccagtactgcttttaacaaaatacttcactttcatcgatgctgtttttgcacgctgcccttgtgtcgttccactaaagcttaaatcgtcaatacttgcaattttgacagaatcagcgcttgtttctttatttttcaagatccagttcttaactccggaaagtcttgcagaatcagtttcagtcgctgaagaccaccaagaagaaggcttcgttaagtccaaagatttcgtatctagctgttgttttgacaaagtaagcgtccagcctatttgaggatcctttgtatctttttttgcaatcaagtaaggtacacttgacgaccacacttcattactagcttctgtgtaaccaccattactggaagaataagctgcagttataagagatccattatatttcagaactttccccttcgtttgctctaccgctttattggtattcgaattccaactgtagccaccataaacttgaaaagcggttgtatcaggtacagtagttcctgtttttgttattgagtaggttcttgcagcgacagtctgtgctttaagagcctcaagtgaccagctagcaggcatttcattcggaattactccttttaagtaatcctcaaatggaatattctcatttaccggtcggatatatttagtagactcaatagagaaattaaccgtgccaagatactgctttccatcaagactaattctgtttgaagtagaatagttttctggtttaattcttagggagtttccgtaagttttgatgttttcaaggtttaattttccgctgttgatttttagattataggtgccaccgtcagctaaataaaattcatcagcaagtgaattctcaacttcagcagaaattgaactggtacttccaataaagtcgtatgcatatgtggctttatctttcgttatacttttagccgcagccggaacgctatcttgtttaacaaataaaatttggctgtttttcttcgaggcaagcgaagcgcctatcaatacgtctgcatattttgtcccgttcgtcattacgactttatcagccttcaactttagttgcttaattatatttgccgtaagttcatatctggttgatccaggaatcctttgcacagttgaagtttttttaatttggttttctacactatcactgacgctaaatgaactgcctataatgatgacttgttttggcaaatcataatccggaagcttgtctttctctgtaagcaaaatagggtatccgtgtgctgctgcatatggagcaatggctaaagcgtcttgaaatactcttccggttacaacgatcgctttatcatagctgcccatttgctttgcaatattttctgataagacatatctattttttccgcttatccttttcacagcgccatatgatttcagcttgttttctacgtcttttgaaatgcttcgcgctcccccaatgattaaaatattatcagggttaaacttagcaatttgtctttcagtagtttttgttaacgtgtcaggctgagtaaataaaactggtgcgttcagttttttcgccaatggaataacaggcaatgcatcaataaaaatatctcggttcaccaatatcactgtattaggatttttccactggctgttggatgcaagagtcgcagtttcgtatcgtgaagcgcctgcaaaacgatccgtaacagcaacattatctccagttactttataaaatcctgttggcgatagactgatgctagatttatttccaatataatttaacaatttaactgagatgtttgagtctgctgcaaaagaaactgatggaatcaataataagattgcagcaagtgaacacactatcaattgtttgcaagatttcaa

SEQ ID NO: 34 - DNA, 1491 bp, Bacillus subtilis
  source 1..1491: mol_type = genomic DNA
ttatctgtaataagatactgtgccgtcatgaatagcttgtgcagctttatctttataaaccgcttgcttcaatttacttgcatctgatgcattagtgataaaggcagtttcaactaaaacactcggcattttagaatatttaataacatagaaagcagctgtttttactccccggtctctcgttccaagattagccgctaactttggttgaatttgttcagccagtctcttgctatttgcagcttgatatgttgtatcgtagtacgtctcacttccatttggtgatgagctatcattagcatttgcatgtatactgagaaataaatctgcttgtgcagaagctgctttatttactctctcctgtaaagaataaaaagtatcattagatcttgacagtactggaagagcacctgaagcatttagctttgtattgactcttttcgctatatcaaggttgacttctttctcaaggagtccattgccgattgctcctgaatcttgatcaccgtgacccggatcaataaagattgtttcacctacaactggattctttagctgattagcaacctttgtgcttacggcaggagtgtttccgataatcataaagtttgacatgtttttacttccaataattttacgggctcctgtagataaattttcaccatttgtaagaataagagattgcttcttcttagctgccagtgtagctcctgcaatagagtcagggtagctgaatccattgcttacatatacggtgcttgttgataaattaagtttttgtacgatatttgcagcaagctcatatctgtttgaaccgctaattcttgtaggagaaggtaacttgttgtataccgtattgctgatacttccagtgcctcctacaacaacggtactcgaaattcccttatcttttatcacagacgtagtcgcactatttatagatgttttatttgtaaaaagaattggatacccgtttttcgctgcataagggatgacggccggagcgtctgcatataagaagccgttcaaaataacagcttttgaagtcgcacccatcgcttttgccacccgtgcagccgtatcataacggttgcttcctgcaattcgtttaatacttatccccaagcttttaatctggttagcagtgttagaagaaacagcaggtgttccgcctacaataattacatttttagtctgcatctctttcaatcttgttttcgtttcatatgaaagcttatcagaattagtgtaaagcaatggcgcattcttctggtaagcaagaggtgctgctgaaatagcatctgcataggaactcccaccaacaattacagctgtacttgctgttgaatacatttgctttgatatttgtacagcagtgccgtatctattgcttcccccaactcttttcactgagttatcggccaaagctgttggcacaaaaagtatgagccccagaaaacacattgttaggacttttatataagaacgcaa

SEQ ID NO: 35 - DNA, 1005 bp, Bacillus subtilis
  source 1..1005: mol_type = genomic DNA
atgaaaaagcaaatcattacagctacgacagcagttgttttaggatcgacgttatttgcaggagcggcatctgcacaaagcattaaggtgaaaaaaggcgacacgttatgggatctttcaagaaaatacgacacaacgatcagtaaaattaaatcagagaaccaccttcgttcagacattatttatgtgggacaaactttatcgattaacggcaaatctacaagttcaaaaagcagcagttcttcttcctcttcttctacatacaaagtaaagagcggggacagcctttggaaaatttcaaaaaaatacggcatgacaatcaatgaactgaagaagctgaatggcttaaaatcagatttgcttcgtgttggacaagtcctgaaactgaaaggttcaactagttcaagcagctccagctcatcaaaagtgtcatcgtcttcaacttctacttataaagtgaagagcggagacagcctttctaaaattgcgagcaaatacggcactacggttagcaaattaaaaagcttaaacggcttaaaatcagatgtaatctatgtcaaccaagtattgaaggtgaaaggaacaagcacaagcagctcaaaacctgcttcatcttcatcgtcttcaagcagcaaaacgtcatctacatcacttaatgtgagcaagctggtttctgatgcaaaagcgttagtcggaacgccatataaatggggcggaacgacaacttcaggctttgactgcagcggattcatttggtacgtactgaataaacaaacaagtgtgggcagaacaagcactgcaggatactggagttctatgaagagcattgccagcccgtctgttggtgatttcgtcttcttcacaacatataaatccggcccttctcacatggggatttacattggaaacaacagtttcattcatgcaggatctgacggcgtacaaatcagcagcctgaacaacagctactggaagcctcgttacctcggtgcgaaaagattctaa

SEQ ID NO: 36 - DNA, 1104 bp, Bacillus subtilis
  source 1..1104: mol_type = genomic DNA
atgtcagttttcactaatagctacattccagtcaataagtatactagaccaggtttgaaattacagggtgtgaaaaaatgcgtcctacactatacagccaatccgggtgcaggtgcagacaatcatcgaagatacttcagtaatgcacaagtttatgcatcagctcacattttcgtagataaggctgaagcaatttgtatcattccattaaatgaagtagcttaccatgcaaatgatattcagcaaagagatagtgccggaaatccttatcgaggagttgctgcgctgaaacctaacgctaactttctttctattggagttgaaatgtgccttgaaaaagacggttcattccattcagatacagttgaaagaactgaggatgtgttcgttgaattatgtaataagtttggtttagatcctattgatgatattgttcgtcattatgacatcacccataagaattgccctgcaccatgggtatctaacagccaaaaatttgtagactttaaaaatcgagtaaaggcaaaaatgtcaggcaaatctgtttcaaaagcttctccaactaaaccaacaacctcctccccttcctcttcatcagcagtaagtggttcactaaaatcaaaagttgacggacttcgcttctattcaaaaccatcttgggaagataaagatgttgtcggcacagtaaataaaggcatcggatttcctacagttgtagagaaagttaaagttggatctgcctatcaatacaaagttaagaactcaaaaggcactacatattacatcactgcttctgacaaatatgttgatgttacaggatcagttaaaacctcttcctctgccccaaaaacaacatcaacttcttcaagttcctcatctattaaatccgtaggaaaaatcaaaattgtcggtgtatcaagcgctgcaatcgtaatggacaagcctgatcgaaatagttctaaaaatattggcacagttaagcttggaagcactatttcaatttctggttcagttaaaggtaaaaacaattccaatggctactgggaagttatttataaaggtaaacgtggatatatctcgggacagtttggatcaacaatctaa

SEQ ID NO: 37 - DNA, 213 bp, Bacillus subtilis
  source 1..213: mol_type = genomic DNA
atggaaatggatataacacaatatttaagtacccaggggccatttgctgttttattttgttggctacttttctacgtaatgaaaactagtaaggaaagagagtcgaaactttataatcaaatcgattctcaaaacgaagtactgggtaaattcagtgaaaagtacgatgttgtaattgaaaagctagataaaatcgaacaaaattttaagtag

SEQ ID NO: 38 - DNA, 267 bp, Bacillus subtilis
  source 1..267: mol_type = genomic DNA
atgtttgagaatattgataaaggcacaattgttaggactcttttgctcgcaatagctttactcaatcaaataatggtgatgctgggtaaagcagcattcatcattaacgaagaggacataaatcatttatatgattgtttatatacaattttcactatcgtcttcacaaccagtactactaccgcagcatggttcaaaaacaattacataactgcaaaaggaaaaaaacaaaaacaagttctaaaaaaagagaacttgtttaaatag

SEQ ID NO: 39 - DNA, 819 bp, Bacillus subtilis
  source 1..819: mol_type = genomic DNA
atggccattaaagttgtaaagaatctagtctctaaatcaaagtatggattgaaatgtcctaatccaatgaaagctgaatatatcactattcataacactgcgaatgatgcttcagcagccaatgagatttcttacatgaagaataactctagctcaacaagttttcactttgcagtagacgataaacaagtcattcaaggtattccaacgaatcgtaacgcttggcacacaggagatggaacaaacggtacagggaatcgcaagtctattggtgtcgaaatttgttatagcaagtcaggaggggtacgatataaggcagcggaaaagcttgctattaagtttgtggctcagctacttaaagaacgtggatggggtattgatcgagtccgcaaacatcaagactggaatggtaagtattgcccgcaccgcattttgtcagagggaagatggattcaagttaagactgcaattgaagcagaattgaaaaagttgggcggaaaaacaaactcaagcaaagcaagtgtagctaaaaagaaaacaacaaacacaagcagcaaaaaaacgtcatatgcgctaccatccggtatttttaaagtgaagagcccaatgatgagaggggaaaaggtaacacaaattcaaaaagcactggctgcactatacttttacccggataaaggagcaaaaaacaacggcattgacggcgtgtatggtccgaaaacagcagatgcaattagacgattccagtctatgtacgggcttactcaagacggtatttacggaccaaaaacgaaagcgaaacttgaagctctcttgaagtaa

SEQ ID NO: 40 - protein, 102 aa, Bacillus subtilis
MKKFIALLFFILLLSGCGVNSQKSQGEDVSPDSNIETKEGTYVGLADTHTIEVTVDNEPVSLDITEESTSDLDKFNSGDKVTITYEKNDEGQLLLKDIERAN

SEQ ID NO: 41 - protein, 705 aa, Bacillus subtilis
MKSCKQLIVCSLAAILLLIPSVSFAADSNISVKLLNYIGNKSSISLSPTGFYKVTGDNVAVTDRFAGASRYETATLASNSQWKNPNTVILVNRDIFIDALPVIPLAKKLNAPVLFTQPDTLTKTTERQIAKFNPDNILIIGGARSISKDVENKLKSYGAVKRISGKNRYVLSENIAKQMGSYDKAIVVTGRVFQDALAIAPYAAAHGYPILLTEKDKLPDYDLPKQVIIIGSSFSVSDSVENQIKKTSTVQRIPGSTRYELTANIIKQLKLKADKVVMTNGTKYADVLIGASLASKKNSQILFVKQDSVPAAAKSITKDKATYAYDFIGSTSSISAEVENSLADEFYLADGGTYNLKINSGKLNLENIKTYGNSLRIKPENYSTSNRISLDGKQYLGTVNFSIESTKYIRPVNENIPFEDYLKGVIPNEMPASWSLEALKAQTVAARTYSITKTGTTVPDTTAFQVYGGYSWNSNTNKAVEQTKGKVLKYNGSLITAAYSSSNGGYTEASNEVWSSSVPYLIAKKDTKDPQIGWTLTLSKQQLDTKSLDLTKPSSWWSSATETDSARLSGVKNWILKNKETSADSVKIASIDDLSFSGTTQGQRAKTASMKVKYFVKSSTGSYNLSKITTISVPTSELRTMIGATVFKSTYVTVKKDTSKYTISGKGYGHGIGMSQYGAKARAEAGDSYSSILKFYYPGTTLTSY

SEQ ID NO: 42 - protein, 496 aa, Bacillus subtilis
MRSYIKVLTMCFLGLILFVPTALADNSVKRVGGSNRYGTAVQISKQMYSTASTAVIVGGSSYADAISAAPLAYQKNAPLLYTNSDKLSYETKTRLKEMQTKNVIIVGGTPAVSSNTANQIKSLGISIKRIAGSNRYDTAARVAKAMGATSKAVILNGFLYADAPAVIPYAAKNGYPILFTNKTSINSATTSVIKDKGISSTVVVGGTGSISNTVYNKLPSPTRISGSNRYELAANIVQKLNLSTSTVYVSNGFSYPDSIAGATLAAKKKQSLILTNGENLSTGARKIIGSKNMSNFMIIGNTPAVSTKVANQLKNPVVGETIFIDPGHGDQDSGAIGNGLLEKEVNLDIAKRVNTKLNASGALPVLSRSNDTFYSLQERVNKAASAQADLFLSIHANANDSSSPNGSETYYDTTYQAANSKRLAEQIQPKLAANLGTRDRGVKTAAFYVIKYSKMPSVLVETAFITNASDASKLKQAVYKDKAAQAIHDGTVSYYR

SEQ ID NO: 43 - protein, 334 aa, Bacillus subtilis
MKKQIITATTAVVLGSTLFAGAASAQSIKVKKGDTLWDLSRKYDTTISKIKSENHLRSDIIYVGQTLSINGKSTSSKSSSSSSSSSTYKVKSGDSLWKISKKYGMTINELKKLNGLKSDLLRVGQVLKLKGSTSSSSSSSSKVSSSSTSTYKVKSGDSLSKIASKYGTTVSKLKSLNGLKSDVIYVNQVLKVKGTSTSSSKPASSSSSSSSKTSSTSLNVSKLVSDAKALVGTPYKWGGTTTSGFDCSGFIWYVLNKQTSVGRTSTAGYWSSMKSIASPSVGDFVFFTTYKSGPSHMGIYIGNNSFIHAGSDGVQISSLNNSYWKPRYLGAKRF

SEQ ID NO: 44 - protein, 367 aa, Bacillus subtilis
MSVFTNSYIPVNKYTRPGLKLQGVKKCVLHYTANPGAGADNHRRYFSNAQVYASAHIFVDKAEAICIIPLNEVAYHANDIQQRDSAGNPYRGVAALKPNANFLSIGVEMCLEKDGSFHSDTVERTEDVFVELCNKFGLDPIDDIVRHYDITHKNCPAPWVSNSQKFVDFKNRVKAKMSGKSVSKASPTKPTTSSPSSSSAVSGSLKSKVDGLRFYSKPSWEDKDVVGTVNKGIGFPTVVEKVKVGSAYQYKVKNSKGTTYYITASDKYVDVTGSVKTSSSAPKTTSTSSSSSSIKSVGKIKIVGVSSAAIVMDKPDRNSSKNIGTVKLGSTISISGSVKGKNNSNGYWEVIYKGKRGYISGQFGSTI

SEQ ID NO: 45 - protein, 102 aa, Bacillus subtilis; identical to SEQ ID NO: 40

SEQ ID NO: 46 - protein, 70 aa, Bacillus subtilis
MEMDITQYLSTQGPFAVLFCWLLFYVMKTSKERESKLYNQIDSQNEVLGKFSEKYDVVIEKLDKIEQNFK

SEQ ID NO: 47 - protein, 88 aa, Bacillus subtilis
MFENIDKGTIVRTLLLAIALLNQIMVMLGKAAFIINEEDINHLYDCLYTIFTIVFTTSTTTAAWFKNNYITAKGKKQKQVLKKENLFK

(19) United States
(12) Patent Application Publication
(10) Pub. No.: US 2023/0407361 A1
(43) Pub. Date: Dec. 21, 2023

(54) INDUCIBLE CELL LYSIS SYSTEM

(71) Applicant: Motif FoodWorks, Inc., Boston, MA (US)

(72) Inventors: Lennart BIERMANN, Stuttgart (DE); Marius HENKEL, Ostfildern (DE); Lars LILGE, Bad Ditzenbach (DE); Rudolf HAUSMANN, Stuttgart-Vaihingen (DE); Philippe PROCHASSON, Boston, MA (US)

(21) Appl. No.: 18/336,868

(22) Filed: Jun. 16, 2023

Related U.S. Application Data
(60) Provisional application No. 63/353,291, filed on Jun. 17, 2022.

(51) Int. Cl. C12P 21/00; C12N 15/75; C12N 1/36

(52) U.S. Cl. CPC C12P 21/00; C12N 15/75; C12N 1/36; C12N 2800/101

(57) ABSTRACT

A programmable cell-lysis system and related compositions and methods are described, which facilitate expression and harvest of recombinant proteins from host cells. The programmable cell-lysis system comprises a host cell comprising a polynucleotide sequence encoding a cell lysis promoting polypeptide or peptide operably linked to an inducible promoter. The cell lysis systems can be configured to produce various heterologous polypeptides for different applications.

### CROSS-REFERENCE TO RELATED APPLICATIONS

This application claims priority from the U.S. Provisional Application No. 63/353,291 filed Jun. 17, 2022. The contents of the aforementioned application is hereby incorporated by reference in its entirety.

### SEQUENCE LISTING

The present application contains a Sequence Listing which has been submitted in .XML format via PatentCenter and is hereby incorporated herein by reference in its entirety. Said WIPO Sequence Listing was created on Jun. 16, 2023 is named 106753_750195_US_SequenceListing.xml and is 82 kilobytes in size.

### FIELD

The present disclosure relates to engineered cells and cell cultures, and more specifically to cells and cell systems engineered to facilitate collection of recombinant proteins from the cells.

### BACKGROUND

Heterologous gene expression in engineered microbial cell lines is a primary means of generating proteins in various industries. Cost-effective, commercial-scale production, harvest and purification of recombinant proteins remain significant challenges. Multiple interconnected factors such as the choice of host cell, protein being expressed, expression system being used, culture system being used, etc. must be balanced against one another, making optimization of any one system complex. Recovering a non-native, heterologous protein at reasonable yield remains a serious challenge. For non-secreted proteins, the cells must be lysed, and the target heterologous protein separated from unwanted cellular components. In addition to incurring considerable time and expense in the target protein recovery process, current methods provide a less than ideal yield. A clear need remains for improved systems that facilitate harvesting of recombinant proteins from host cells.

### SUMMARY

Various aspects of the current disclosure encompass an engineered host cell, wherein the host cell comprises: (a) modification of at least one nucleic acid sequence encoding a sporulation-promoting polypeptide, a cell lysis inhibitor polypeptide or a combination thereof; and optionally, (b) a first polynucleotide sequence encoding at least one cell lysis promoting polypeptide. In some aspects, the engineered host cell comprises at least one cell lysis promoting polypeptide.

In some aspects, the engineered host cell is derived from a Bacillus subtilis 168, 3NA, BMV9, or IIG-Bs-20-1 strain. In some aspects, the at least one nucleic acid sequence of (a) is selected from The engineered host cell of any one of claim 1 or 2, wherein the at least one nucleic acid sequence of (a) is selected from the polynucleotide sequence of sdpR, sdpI, spo0A, sigW, yfhL, yknW, yknX, yknY, yknZ, clpP, arbB or any combination thereof. or any combination thereof. In some aspects, the cell lysis promoting polypeptide is SkfA, SkfB, SkfC, SkfE, SkfF, SkfG, SkfH, SdpA, SdpB, SdpC, SdpI, SdpR, LytA, LytB, LytC, LytE, BlyA, BhlA, BhlB, CwlA, or a derivative or fragment thereof. In some aspect, the polynucleotide sequence encoding the cell lysis promoting polypeptide comprises a nucleic acid sequence having at least 85%, 90%, 95% or 99% sequence identity with a sequence as provided in SEQ ID NOs: 1-12, 32-39 or a fragment or functional derivative thereof. In some aspects, the first polynucleotide sequence comprises a sequence selected from SEQ ID NOs: 1-12, 32-39, or a fragment thereof. In some aspects, the first polynucleotide sequence comprises the nucleic acid sequence encoding a polypeptide having at least 85%, 90%, 95% or 99% sequence identity with SEQ ID NOs: 13-24, 40-47 or a fragment thereof.

In some aspects, the first polynucleotide sequence of (b) comprises a nucleic acid sequence encoding at least 2, or at least 3, or at least 4, or at least 5 or more, cell lysis promoting polypeptides. In some aspects, the first polynucleotide sequence of (b) is operably linked to a first promoter. In some aspects, the first promoter comprises a constitutive or an inducible promoter. In some aspects, the inducible promoter may be a thermosensitive, a chemosensitive, or a photosensitive promoter.

In some aspects, the engineered host cell as disclosed herein may further comprise a second polynucleotide sequence comprising a second nucleic acid sequence encoding a heterologous polypeptide operably linked to a second promoter. In some aspects, the heterologous polypeptide is, for example a nutritive, therapeutic, enzymatic, and/or a food preservative protein. In some aspects, the heterologous polypeptide is selected from casein, a casein subunit, a heme protein, a lactalbumin, a lactoglobulin, an ovalbumin, an ovotransferrin, an ovomucoid, an ovomucin, and a lysozyme. In some aspects, the heme protein is selected from a hemoglobin, a soy leghemoglobin, or a myoglobulin. In some aspects, the heterologous polypeptide comprises α-casein, β-casein, or κ-casein.

In some aspects, the heterologous polypeptide comprises α-lactalbumin.

In some aspects, the second promoter comprises a constitutive or an inducible promoter system. In some aspects, the inducible promoter is, for example, a thermosensitive, a chemosensitive, or a photosensitive promoter.

Examples of chemosensitive promoter include P_{grac}100, P_{spac}, P_{xylA}, P_{licB}, PT7, PT7_{lac}, P_{manP}, and P_{manR}. Examples of suitable constitutive promoter comprises a Pveg promoter.

In some aspects, the engineered host cell may comprise an expression vector, a plasm id vector, a bacteriophage, a transposon, or genomic DNA comprising the first polynucleotide sequence and/or the second polynucleotide sequence. In some aspects, the plasm id vector is a dual expression vector comprising the first polynucleotide sequence and the second polynucleotide sequence.

In some aspects, the current disclosure also encompasses a method of producing a heterologous polypeptide, the method comprising: a) culturing the engineered host cell as disclosed herein; b) expressing the heterologous polypeptide in the host cell. In some aspects, the method further comprises inducing cell lysis by maintaining the host cell for a time and under conditions sufficient for expression of at least one cell lysis promoting polypeptide. In some aspects, the method further comprises harvesting the heterologous polypeptide from culture supernatant. In some aspects, the host cell comprises an inducible promoter and the method further comprises introducing the host cell to conditions that induce the promoter to induce the expression of the heterologous polypeptide. In some aspects, the inducible promoter is a chemosensitive promoter is selected from P_{grac}100, Pspac, PxylA, PlicB, PT7, PT7lac, PmanP, and PmanR. In some aspects of the disclosed method, the culturing of the engineered host cell is performed in a fermenter. In some aspects, the heterologous polypeptide comprises a nutritive, therapeutic, enzymatic, and/or a food preservative protein.

In some aspects, the heterologous polypeptide is selected from casein, a casein subunit, a heme protein, a lactalbumin, a lactoglobulin, an ovalbumin, an ovotransferrin, an ovomucoid, an ovomucin, and a lysozyme. In some aspects, the heme protein is selected from a hemoglobin, a soy leghemoglobin, or a myoglobulin. In some aspects, the heterologous polypeptide comprises α-casein, β-casein, or κ-casein. In some aspects, the heterologous polypeptide comprises α-lactalbumin.

In some aspects, the current disclosure also encompasses an engineered cell lysis system for production of a heterologous polypeptide, the system comprising a host cell as disclosed herein.

In some aspects, the current disclosure also encompasses a culture comprising the host cell as disclosed herein, and a heterologous polypeptide.

In some aspects, the current disclosure also encompasses a method for recovering a heterologous polypeptide produced by a host cell in culture, the method comprising: (a) introducing or having introduced an engineered nucleic acid construct or engineered expression vector comprising a first polynucleotide sequence encoding a cell lysis promoting protein or fragment or derivative thereof, wherein the cell expresses the heterologous polypeptide; (b) inducing expression of the cell lysis protein or peptide in the cell, thereby lysing the cell to form a lysed cell mixture; and (c) harvesting the heterologous polypeptide from the lysed cell mixture. In some aspects, the cell is derived from a Bacillus subtilis 168, 3NA, BMV9, or IIG-Bs-20-1 strain.

In some aspects, the current disclosure also encompasses a method of recovering a heterologous polypeptide produced in a cell culture comprising: (a) culturing the host cell as disclosed herein, under conditions sufficient for expression of the heterologous polypeptide; (b) inducing the expression of the cell lysis protein or peptide thereby lysing the cell to form a lysed cell mixture; and (c) harvesting the heterologous polypeptide from the lysed cell mixture.

Various aspects the present disclosure encompass an engineered nucleic acid construct or engineered expression vector comprising a nucleotide sequence encoding a cell lysis promoting protein or peptide operably linked to an inducible heterologous promoter or to an inducible heterologous promoter system. In some aspects the engineered nucleic acid construct or engineered expression vector comprises a nucleotide sequence has at least 85%, 90%, 95% or 99% sequence identity with a mature polypeptide coding sequence for an SKF and/or SDP protein or peptide. In some aspects the SKF protein or peptide is selected from SkfA, SkfB, SkfC, SkfE, SkfG, and SkfH. In some other aspects the SDP protein or peptide is selected from SdpA, SdpB, and SdpC.

In some aspects the engineered nucleic acid construct or engineered expression vector comprises a mature polypeptide coding sequence as set forth in any one of SEQ ID NOs: 1-12.

In some aspects the current disclosure encompasses engineered nucleic acid construct or engineered expression vector comprising a nucleotide sequence encoding a cell lysis promoting protein or peptide operably linked to an inducible heterologous promoter, wherein the inducible heterologous promoter or inducible heterologous promoter system comprises a thermosensitive, a chemosensitive, or a photosensitive promoter. Non-limiting examples of chemosensitive promoter selected from P_{grac}100, P_{spac}, P_{xylA}, P_{licB}, PT7, PT7_{lac}, P_{manP}, and P_{manR}. In some aspects the heterologous promoter system is a thermosensitive promoter system. In some aspects the thermosensitive promoter system comprises a first promoter operably linked to the polynucleotide encoding the cell lysis promoting protein or peptide, and a second promoter operably linked to a nucleic acid encoding a transcription factor for the first promoter, wherein the second promoter is thermosensitive or controlled by a thermosensitive regulator as in the thermosensitive promoter system comprising a Clp promoter/CtsR repressor system.

In some aspects the current disclosure encompasses an engineered cell lysis system for recovering a heterologous polypeptide produced by a host cell in culture, the system comprising: (a) the nucleic acid construct encoding a cell lysis system; and (b) a second nucleic acid construct comprising a polynucleotide encoding the heterologous polypeptide operably linked to a constitutive promoter system or a second inducible promoter system. The engineered cell lysis system may comprise a dual expression vector system comprising a first expression vector comprising a nucleotide sequence encoding the nucleic acid construct of (a), and a second expression vector comprising a nucleotide sequence encoding the nucleic acid sequence of (b). In some aspects the engineered lysis system has disrupted or modified expression of a gene for example sdpR, sdpI, spo0A, sigW, yfhL, yknW, yknX, yknY, yknZ, clpP, arbB and any combination thereof.

In some aspects the second nucleic acid construct in the engineered cell lysis system comprises an inducible or a constitutive promoter system, wherein inducible promoter system comprises a thermosensitive, a chemosensitive, or a photosensitive promoter. In some aspects the chemosensitive promoter is P_{grac}100, P_{spac}, P_{xylA}, P_{licB}, PT7, PT7_{lac}, P_{manP}, and P_{manR}. In some aspects the promoter is a constitutive promoter for example the Pveg promoter.

In some aspects the second nucleic acid comprises a polynucleotide encoding a heterologous polypeptide comprising a nutritive, therapeutic, enzymatic, and/or a food preservative protein, for example a casein, a casein subunit, a heme protein, a lactalbumin, a lactoglobulin, an ovalbumin, an ovotransferrin, an ovomucoid, an ovomucin. In some aspects the heterologous polypeptide is a lysozyme, or a hemoglobin, a soy leghemoglobin, or a myoglobulin. In some aspects the heterologous polypeptide comprises α-Casein, β-Casein, or κ-Casein. In some other aspects the heterologous polypeptide comprises α-lactalbumin.

In some aspects the current disclosure also encompasses host cell comprising the nucleic acid construct or expression vector disclosed herein and/or the engineered cell lysis system disclosed herein. In some aspects the host cell has disrupted or modified expression of one or more of the genes encoding the SDP proteins, SKF proteins, and any combination thereof. In some aspects the host cell is a non-sporulating cell with disrupted or modified expression of one or more of the genes regulating the expression of the SDP and/or the SKF proteins. Examples of SDP proteins include SdpA, SdpB, SdpC, SdpR, SdpI. Examples of SKF proteins include SkfA, SkfB, SkfC, SkfE, SkfF, SkfG, SkfH and combinations thereof. In some aspects the host cell is a Bacillus subtilis cell. In some aspects the host cell is derived from Bacillus subtilis 3NA. In some aspects the host cell is derived from Bacillus subtilis BMV9.

In some aspects the current disclosure encompasses a culture comprising the host cells disclosed herein.

In some aspects the current disclosure encompasses a method for recovering a heterologous polypeptide produced by a cell in culture, the method comprising: (a) introducing or having introduced an engineered nucleic acid construct or engineered expression vector comprising a nucleotide sequence encoding a cell lysis promoting protein or peptide, wherein the cell expresses the heterologous polypeptide; (b) inducing expression of the cell lysis protein or peptide in the cell, thereby lysing the cell to form a lysed cell mixture; and (c) harvesting the heterologous polypeptide from the lysed cell mixture.

In some aspects the current disclosure encompasses a method of recovering a heterologous polypeptide produced in a cell culture comprising: (a) culturing the host cell disclosed herein under conditions sufficient for expression of the heterologous polypeptide; (b) inducing the expression of the cell lysis protein or peptide thereby lysing the cell to form a lysed cell mixture; and (c) harvesting the heterologous polypeptide from the lysed cell mixture. In some exemplary aspects the heterologous polypeptide comprises a nutritive, therapeutic, enzymatic and/or a food preservative protein. In some aspects the heterologous polypeptide comprises a protein for example casein, a casein subunit, a heme protein, a lactalbumin, a lactoglobulin, an ovalbumin, an ovotransferrin, an ovomucoid, an ovomucin, or a lysozyme. In some aspects the heterologous polypeptide comprises hemoglobin, soy hemoglobin, or myoglobulin. In some exemplary aspects the heterologous polypeptide comprises α-casein, β-casein, or κ-casein. In some aspects the heterologous polypeptide comprises α-lactalbumin. In some aspects the current disclosure encompasses a host cell having a modified expression of a gene selected from sdpR, sdpI, spo0A, sigW, yfhL, yknW, yknX, yknY, yknZ, clpP, arbB and any combination thereof. In some aspects the host cell further comprises an engineered nucleic acid construct or engineered expression vector comprising a nucleotide sequence encoding a cell lysis promoting protein or peptide operably linked to an inducible heterologous promoter or to an inducible heterologous promoter system. In some aspects the cell lysis promoting protein or peptide may include SkfA, SkfB, SkfC, SkfE, SkfF, SkfG, SkfH, SdpA, SdpB, SdpC, SdpR, SdpI, and any combinations thereof.

In various aspects of the cell lysis systems, host cells and methods disclosed herein, expression of a gene is modified, the host cell is a bacterial and the modified gene can be clpP or clpE. In a non-limiting example, the host cell is Bacillus subtilis and the expression of clpP is modified.

### BRIEF DESCRIPTION OF THE FIGURES

FIG. 1 is a schematic illustration providing an overview of a Bacillus subtilis sporulation and cannibalism process (Gonzalez-Pastor, J. E. (2011).

FIG. 2 is a schematic illustration of genes encoding cannibalism factors and native operons or regulatory sequences in Bacillus subtilis (González-Pastor, J. E. (2011).

FIG. 3A shows the plasmid map of pHT254-αS1-Casein, with casein gene linked with a C-terminal His-tag cloned under control of the P_{grac}100 promoter.

FIG. 3B shows the plasmid map of pHT1469-αS1-Casein, with the C-terminally His-tagged casein gene including an N-terminal amyQ leader sequence for secretion cloned under the P_{grac}100 promoter.

FIG. 3C shows the plasmid map of pDG148-Stu_Casein, with the C-terminally linked casein gene cloned under the IPTG-inducible P_{spac} promoter.

FIG. 3D shows the plasmid map of pMutin_Casein, with the C-terminally linked casein gene cloned under the IPTG-inducible P_{spac} promoter.

FIG. 4 is a schematic illustration of a casein expression system using a chromosomally integrated T7 expression system wherein the T7 polymerase gene is cloned under a native constitutive promoter and casein gene expression is under a T7 promoter.

FIG. 5A Casein expression from pHT254-αS1-Casein from different E. coli strains (lanes 2, 6, 7, 9) after induction with 1 mM IPTG.

FIG. 5B Purification of the αS1-casein after IPTG-mediated expression using the pDG148-Stu_casein expression system. Casein was purified using immobilized metal affinity chromatography.

FIG. 6A Schematic illustration of the biosynthesis and maturation process of SDP killing factor.

FIG. 6B Schematic illustration of the biosynthesis and maturation process of SKF killing factor.

FIG. 7A shows the plasm id map of pDG148-Stu_sdpABC, with the sdpABC gene cluster cloned under the control of IPTG-inducible P_{spac} promoter.

FIG. 7B shows the plasmid map of pDG148-Stu_skfABC, with the skfABC gene cluster cloned under the control of IPTG-inducible P_{spac} promoter.

FIG. 7C shows the plasmid pMutin-skfABCEFGH, with the skfABCEFGH operon cloned under the control of IPTG-inducible P_{spac} promoter.

FIG. 8A shows the process of the Gibson cloning using the pJOE6732.1 plasmid for the marker less promoter exchange of the native Pₛₖₚ against the heat-inducible P_{clpE} promoter.

FIG. 8B shows the Gibson cloning using the pJOE6732.1 plasmid for the markerless promoter exchange of the native P_{sdp} against the heat-inducible P_{clpE} promoter

FIG. 8C shows the requirements for the removal of the resistance marker after chromosomal integration using pJOE6732.1 plasmid, which encodes the Cre recombinase. After recognition of the flanking lox-sites (lox71 and lox66), the middle sequence segment is removed, resulting in a final lox72 site.

FIG. 9 shows a general schematic of the cell lysis plate assay.

FIG. 10 are photographs showing the results of a plate assay where an IPTG-inducible sdpABC overexpression system was used for overnight production of the SDP killing factor on agar plates, but in a background strain with sigW deletion. The corresponding indicator strains are specified in the figures. The halo indicates the efficiency of the SDP killing factor in relation to the indicator strain.

FIG. 11A is a schematic showing the effect of AbrB on cell lysis. AbrB is an inhibitor of cell lysis, that is regulated by Spo0A. Deletion of AbrB can potentially enhance cell lysis.

FIG. 11B provides photographs showing the results of a plate lysis assay with strains deleted for spo0A or abrB or both. Cells deleted for abrB show a clear zone of clearance as shown in the figure. The strains used in the experiment are specified in the figure.

FIG. 11C provides the growth curves for B. subtilis 168 ΔabrB pDG148-sdpABC in LB-Medium. The cells were induced after 3 hrs (gray). An uninduced sample was used as control (black).

FIG. 12 provides a photograph of the plate assay conducted with the reduced genome IIG-Bs-20-1 (B. subtilis 168). A clear inhibition zone could be seen around the strain, indicating at at least some of the genes deleted impact cell lysis in comparison to the control (B. subtilis 168).

FIG. 13 is a schematic overview of Sdp killing factor formation and the impact of SdpC on the various deletion mutant strains tested.

FIG. 14A is a schematic of the plate lysis assay conducted for mass spectrometric analysis for lysis genes.

FIG. 14B provides photographs of the plate lysis assay for mass spectrometric analysis. The marked areas were cut out and sent for proteome analysis.

FIG. 15A provides a schematic of the stages of processing of the SdpC polypeptide (Pérez Morales 2013).

FIG. 15B provides a growth curve at the exponential phase of growth for the strain used for purification of SdpC (n=2). The experimental culture was induced with 1 mM IPTG after 3 hrs and growth was monitored up to 9 hrs (Cells were grown at 37° C., with shaking at 120 rpm). Cell lysis in the medium would result in reduction of the optical density at 600 nm.

FIG. 15C provides a growth curve at the stationary phase of growth for the strain used for purification of SdpC (n=2). The experimental culture was induced with 1 mM IPTG after entering stationary phase (37° C., shaking at 120 rpm). Cell lysis in the medium would result in reduction of the optical density at 600 nm.

FIG. 15D is a Coomassie stained SDS-PAGE analysis of secreted proteins after induction in stationary growth phase and exponential growth phase as provided in FIGS. 15B and 15C. Highlighted regions correspond to the area on the gel where toxin/precursors should be visible or detectable. (C=Control; I=Induced)

FIG. 15E is a silver stained SDS-PAGE analysis of the culture supernatant after concentration. Samples were concentrated from 600 mL to 30 mL and loaded on a gel. Highlighted regions correspond to the area on the gel where the protein should be visible or detectable.

FIG. 15F provides a photograph of a plate lysis assay using 2 μL of concentrated supernatant onto BMV9 ΔsdpABC-IR ΔsigW (softagar).

FIG. 15G is a plasmid map of the construct for use in expression and purification of a His-tagged version of SdpC.

FIG. 15H is a plasmid map of the construct for use in expression and purification of an un-tagged version of SdpC.

FIG. 16A is a schematic of a potential promoter exchange construct using the P_{clpE} for heat inducible lysis system for expression of LytB and LytC.

FIG. 16B is a schematic of a potential promoter exchange construct using the P_{mtiA} for mannitol inducible lysis system for expression of LytB and LytC.

FIG. 17 is the plasmid map for pHT254_sdpABC with a stronger promoter P_{grac}100100.

### DETAILED DESCRIPTION

The present disclosure provides compositions and methods relating to an engineered, inducible cell lysis system. Disclosed are compositions including engineered nucleic acid constructs, engineered expression vectors, and systems and recombinant cell lines using them, and methods of use thereof, to achieve inducible cell lysis in a heterologous expression system.

The present disclosure describes new and improved recombinant systems for achieving high protein expression and efficient recovery of heterologous polypeptides from host cells. The disclosure is based in part on the surprising discovery that a defence mechanisms in sporulating bacteria can be engineered to provide for inducible cell lysis in a heterologous expression system, resulting in more efficient recovery of non-secreted heterologous polypeptides from the host cells.

I. Compositions

In one aspect, the present disclosure provides engineered nucleic acid constructs, engineered expression vectors, engineered systems and recombinant cell lines to provide for inducible cell lysis in a heterologous expression system.

One aspect of the present disclosure provides engineered nucleic acid constructs or engineered expression vectors comprising a polynucleotide including a nucleotide sequence encoding a cell lysis promoting protein or peptide, operably linked to an inducible heterologous promoter system. The inducible promoter can be chosen so that cell lysis of a cell comprising the engineered nucleic acid construct or expression vector can be carefully controlled.

(i) Cell Lysis Promoting Factors

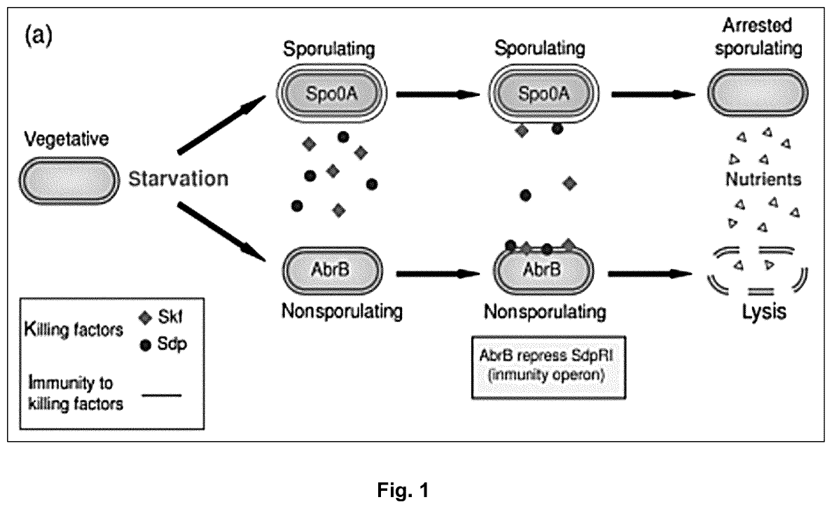

The sporulation process is a defense mechanism used by certain bacteria such as Bacillus subtilis in response to stress. Sporulation is an energy intensive process and may be disadvantages to the fitness of a bacterial population. Bacterial populations have an ingenious mechanism to avoid early commitment to sporulation. Entry into the sporulation process is governed by a regulatory protein Spo0A, that also regulates the skf (sporulation killing factor) and sdp (sporulating delay factor) operons. Additional regulators such as abrB, which encodes a repressor that controls the expression of genes involved in starvation-induced processes such as sporulation and the production of antibiotics and degradative enzymes can also impact sporulation. Restricted nutrients trigger expression of various proteins of the skf and sdp operon. The secreted SKF killing factor triggers cell lysis of neighboring susceptible cells. Without being bound by theory, it is thought that the SDP killing factor may cause a delay in commitment to sporulation in the host cell. In this context, “susceptible cells” are cells that do not express “immunity factors” also encoded by the skf operon, which are tied to the expression of the other SKF/SDP proteins. FIG. 1 is a schematic diagram of this process often called as the cannibalism stress response. As shown in FIG. 1, expression of these “cell lysis” proteins or peptides is normally under the control of an Spo0A master regulator that triggers the expression of these operons. Together, these events delay sporulation in the non-susceptible cells while allowing them to utilize nutrients made available by lysis of surrounding susceptible cells. Table A provides a list of genes in the two operons and the encoded proteins.

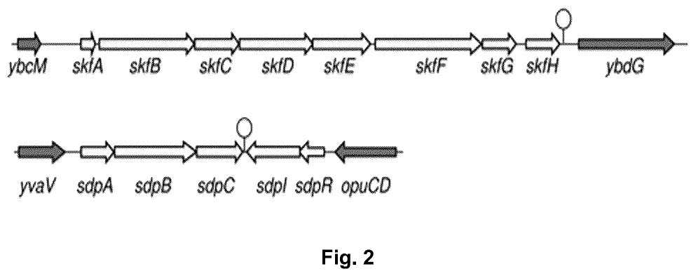

In various aspects, a cell lysis promoting factor comprises a sporulation killing factor (SKF) or a sporulation delaying protein (SDP) or proteins that impact the expression of these factors. Non-limiting examples of a cell lysis promoting protein or peptide are the SKF and SDP proteins expressed by Bacillus subtilis. Table A provides a list of genes in the two operons and the encoded proteins and FIG. 2 is a schematic illustration showing the genetic organization of the SKF (e.g., skfA, skfB, skfC, skfE, skfF, skfG, and skfH) and SDP (sdpA, sdpB, sdpC, sdpI, sdpR) operons in B. subtilis.

TABLE A
List of genes and encoded proteins by the SDP and SKF operons

| Gene | NCBI Gene ID | SEQ ID NO. | Uniprot ID | SEQ ID NO. |
|---|---|---|---|---|
| sdpA | 938629 | 1 | O34889 | 13 |
| sdpB | 936240 | 2 | O34616 | 14 |
| sdpC | 936227 | 3 | O34344 | 15 |
| sdpR | 936249 | 4 | O32242 | 16 |
| sdpI | 936238 | 5 | O32241 | 17 |
| skfA | 938506 | 6 | O31422 | 18 |
| skfB | 938501 | 7 | O31423 | 19 |
| skfC | 8302931 | 8 | O31425 | 20 |
| skfE | 938498 | 9 | O31427 | 21 |
| skfF | 938494 | 10 | O31428 | 22 |
| skfG | 938502 | 11 | O31429 | 23 |
| skfH | 938499 | 12 | O31430 | 24 |

In various aspects, a cell lysis promoting factor of the current disclosure also encompasses polypeptide or RNA effector that impacts the expression and/or functioning of the SDP and SKF proteins. These may include but are not limited to chaperones (for example CsaA), maturation factors (for example signal peptidases like SipS and/or SipT), proteins effectors for posttranslational modifications (for example disulfide bond isomerases like BdbB or BdbC), secretory pathway proteins (for example SecA, SecY, SecE, SecG) etc. Specifically, the pre-version of SDP killing factor is targeted intracellularly by the chaperon CsaA before SDP is secreted by the general secretion system of B. subtilis. Extracellularly, truncation of pre-SDP is facilitated by the signal peptidases SipS and SipT. In addition, SdpA and SdpB are involved in post-translational activation of SDP killing factor via an unknown mechanism.

In some aspects, the lysis promoting factor maybe a protein or a set of proteins that are involved in cell wall lysis. Non-limiting examples of such proteins include proteins encoded by the lytABC operon, various holin-like proteins that regulate cell lysis, for example bhlA and bhlB, and autolysins like cwlA. The lytABC operon encodes the LytABC autolysin complex that specifically sensitizes motile cells to killing by monovalent cations. LytC encodes a critical peptidoglycan hydrolase that mediates autolysis of vegetative cells. LytE mutants exhibit reduced cell lysis. LytD and LytG encode peptidoglycan hydrolases. In one aspect, the current disclosure also encompasses overexpression and/or heterologous expression of one or more genes of the lytABC operon to promote lysis. In one aspect, the current disclosure also encompasses overexpression and/or heterologous expression of one or more genes additional genes for example bhlA, bhlB and cwlA. In some aspects, the current disclosure also encompasses expression or heterologous expression of one of more of SDP/SKF proteins, LytA, LytB, LytC, LytE, BlyA, BhlA, BhlB, CwlA or any combination thereof, to promote lysis.

TABLE B
Additional genes that promote lysis

| Gene | NCBI Gene ID | SEQ ID NO. | Uniprot ID | SEQ ID NO. |
|---|---|---|---|---|
| lytA | 936784 | 32 | Q02112 | 40 |
| lytB | 936795 | 38 | Q02113 | 41 |
| lytC | 936777 | 34 | Q02114 | 42 |
| lytE | 939269 | 35 | P54421 | 43 |
| blyA | 939130 | 36 | O31982 | 44 |
| bhlA | 939132 | 37 | O31983 | 45 |
| bhlB | 939127 | 38 | O31984 | 46 |
| cwlA | 937772 | 39 | P24808 | 47 |

In some aspects the cell lysis promoting factor could be a nucleotide sequence that impacts the expression of the SDP/SKF, LytA, LytB, LytC, LytE, BlyA, BhlA, BhlB, CwlA proteins or any combination thereof. These may include but are not restricted to enhancers, UTRs etc.

(ii) Heterologous Polypeptides

A goal of recombinant production of a protein of interest in a host cell, e.g., a bacterial cell, is to achieve commercially relevant quantities of the protein of interest (POI). Recombinant protein expression is generally accomplished by constructing an expression construct or cassette which includes a nucleotide sequence (DNA) encoding the protein, operably linked to a promoter from a regulated gene, wherein the promoter controls expression of the protein. The host cell is transformed, for example by introducing the expression construct or cassette into the host cell using a vector such as a plasmid. The thus transformed host cell is cultured for a time and under culture conditions sufficient for the protein to be expressed under the control of the promoter. Methods, vectors, and cloning techniques for preparing and introducing exogenous nucleic acid sequences (e.g., those encoding a recombinant protein) into cells are well-known (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, NY, 3rd edition, 2001).

In this context, the inducible cell lysis system provided herein is designed to attain a high level of protein yield and recovery of a non-secreted recombinant protein. Accordingly, the cell lysis system can comprise a polynucleotide comprising a nucleotide sequence encoding any heterologous polypeptide of interest. Although the heterologous polypeptide of interest may be any protein, in various aspects the present disclosure contemplates that the heterologous polypeptide may comprise a nutritive protein, (e.g., a protein that can be added or incorporated into an edible, nutritional composition), enzymes, a therapeutic protein, and/or a food preservative protein. By way of non-limiting examples, a heterologous polypeptide may consist of or comprise a casein, a casein subunit such as an α-casein, a β-casein or a k-casein, a heme protein, a lactalbumin, a lactoglobulin or any egg white protein such as an ovalbumin, an ovotransferrin, an ovomucoid, an ovomucin, and a lysozyme. In various aspects, a heme protein may be any hemoglobin, such as a mammalian hemoglobin or a plant hemoglobin such as soy leghemoglobin; or any myoglobin, such as a mammalian myoglobin or a fish myoglobin. In various aspects, the heterologous polypeptide may comprise an α-lactalbumin. It will be understood that a single protein of interest may exhibit any two or more of nutritive, therapeutic and food preservative functions. For example, ovalbumin or ovomucoid may each be of interest as a nutritive protein and also as a tumor suppression (anti-cancer) agent; ovotransferrin may be of interest as a nutritive protein and as an antimicrobial agent, or an anticancer agent; lysozyme may be of interest as a food preservative.

In various aspects, the heterologous polypeptide can comprise a casein (e.g., a alpha-Casein). In various aspects, the alpha-Casein can comprise an amino acid sequence having at least 85%, at least 90%, at least 95%, at least 96%, at least 97%, at least 98%, or at least 99% sequence identity to SEQ ID NO: 26. For example, the alpha-Casein can comprise an amino acid sequence comprising SEQ ID NO: 26. In various aspects, the alpha-Casein can be encoded by a nucleotide sequence having at least 85%, at least 90%, at least 95%, at least 96%, at least 97%, at least 98%, or at least 99% sequence identity to SEQ ID NO: 25. For example, the alpha-Casein can be encoded by a nucleotide sequence comprising SEQ ID NO: 25.

In some other aspects, the heterologous polypeptide/protein of interest (POI) may be any other protein. In some aspects, a POI or a variant thereof may be an enzyme for example acetyl esterases, aminopeptidases, amylases, arabinases, arabinofuranosidases, carbonic anhydrases, carboxypeptidases, catalases, cellulases, chitinases, chymosins, cutinases, deoxyribonucleases, epimerases, esterases, α-galactosidases, β-galactosidases, α-glucanases, glucan lysases, endo-β-glucanases, glucoamylases, glucose oxidases, α-glucosidases, β-glucosidases, glucuronidases, glycosyl hydrolases, hemicellulases, hexose oxidases, hydrolases, invertases, isomerases, laccases, ligases, lipases, lyases, mannosidases, oxidases, oxidoreductases, pectate lyases, pectin acetyl esterases, pectin depolymerases, pectin methyl esterases, pectinolytic enzymes, perhydrolases, polyol oxidases, peroxidases, phenoloxidases, phytases, polygalacturonases, proteases, peptidases, rhamno-galacturonases, ribonucleases, transferases, transport proteins, transglutaminases, xylanases, hexose oxidases, and combinations thereof. In some aspects, a POI or a variant POI thereof is an enzyme selected from Enzyme Commission (EC) Number EC 1, EC 2, EC 3, EC 4, EC 5 or EC 6.

In some aspects, the heterologous polypeptide of interest could be a therapeutic protein for example an antibodies, vaccines, blood factors, protein scaffolds, fusion proteins, anticoagulants, growth factors, hormones, interferons, interleukins, and thrombolytics.

The nucleic acid sequence encoding the heterologous polypeptide may optionally be linked to a selectable marker, such as a nucleic acid sequence encoding hypoxanthine-guanine phosphoribosyltransferase (HPRT), dihydrofolate reductase (DHFR), and/or glutamine synthase (GS), wherein any of HPRT, DHFR, and/or GS can be used as an amplifiable selectable marker. The nucleic acid sequence encoding the heterologous polypeptide may optionally be codon optimized to facilitate and/or promote expression in a heterologous system (i.e., a bacterial system).

(iii) Engineered Cell Lysis Constructs and Vectors

In some aspects the current disclosure encompasses engineered nucleic acid constructs that facilitate controlled expression of cell lysis promoting proteins or peptides.

Accordingly, in various aspects, an engineered nucleic acid construct or engineered expression vector can comprise a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a mature polypeptide coding sequence for an SKF and/or SDP protein or a fragment of derivative thereof. In various aspects, the polynucleotide comprises a nucleotide sequence having at least 85%, at least 90%, at least 95%, or at least 99% sequence identity with a mature polypeptide coding sequence for an SKF and/or SDP protein or a fragment of derivative thereof.

In various aspects, the SKF protein or peptide can be selected from SkfA, SkfB, SkfC, SkfE, SkfF, SkfG, SkfH and any combinations thereof. In further aspects, the SDP protein or peptide can be selected from SdpA, SdpB, SdpC, SdpI, SdpR, and any combinations thereof.

Accordingly, in various aspects the engineered nucleic acid construct or engineered expression vector comprises one or more nucleotide sequences having at least 85%, at least 90%, at least 95%, or at least 99% sequence identity to any one or more of SEQ ID NOs: 1 to 12. In various embodiments, the engineered nucleic acid construct or engineered expression vector comprises any one or more of SEQ ID NOs: 1 to 12.

In various aspects, an engineered nucleic acid construct or engineered expression vector can comprise a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a polypeptide coding sequence for LytA, LytB, LytC, LytE, and/or BlyA (LYT system) or a fragment or derivative thereof. In various aspects, the polynucleotide comprises a nucleotide sequence having at least 85%, at least 90%, at least 95%, or at least 99% sequence identity with a mature polypeptide coding sequence for an LytA, LytB, LytC, LytE, BlyA protein (LYT system) or a fragment or derivative thereof. In some aspects, an engineered nucleic acid construct or engineered expression vector can comprise a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to a polypeptide coding sequence for BhlA, BhlB, and/or Cwl. In some aspects, the engineered nucleic acid construct or engineered expression vector can comprise a nucleotide sequence having at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% to one or more of polypeptide coding sequences for SkfA, SkfB, SkfC, SkfE, SkfF, SkfG, SkfH, SdpA, SdpB, SdpC, SdpI, SdpR, LytA, LytB, LytC, LytE, BlyA, BhlA, BhlB, Cwl or any combination thereof.

Accordingly, in various aspects the engineered nucleic acid construct or engineered expression vector comprises one or more nucleotide sequences having at least 85%, at least 90%, at least 95%, or at least 99% sequence identity to any one or more of SEQ ID NOs: 32 to 39. In various embodiments, the engineered nucleic acid construct or engineered expression vector comprises any one or more of SEQ ID NOs: 32 to 39.

In some aspects, the engineered nucleic acid construct or engineered expression vector comprises at least one, at least two, at least three, at least 4, at least or more of the nucleotide sequence at least about 85% identical to SEQ ID NO: 1-12, SEQ ID NOs: 32-39 or any combination thereof. In some aspects, the engineered nucleic acid construct or engineered expression vector comprises at least one, at least two, at least three, at least 4, at least 5, or more of the nucleotide sequence of SEQ ID NO: 1-12, SEQ ID NOs: 32-39 or any combination thereof.

In various aspects, the engineered nucleic acid construct or engineered expression vector comprises a nucleotide sequence encoding for a polypeptide having at least 85%, at least 90%, at least 95%, or at least 99% sequence identity to any one of SEQ ID NOs: 13 to 24 or a biologically active fragment or derivative thereof. In various aspects, the engineered nucleic acid construct or engineered expression vector comprises a polynucleotide sequence encoding for a polypeptide having an amino acid sequence comprising any one of SEQ ID NOs: 13 to 24 or a biologically active fragment or derivative thereof. In various aspects, the engineered nucleic acid construct or engineered expression vector comprises a nucleotide sequence encoding for a polypeptide having at least 85%, at least 90%, at least 95%, or at least 99% sequence identity to any one of SEQ ID NOs: 40 to 47 or a biologically active fragment or derivative thereof. In various aspects, the engineered nucleic acid construct or engineered expression vector comprises a polynucleotide sequence encoding for a polypeptide having an amino acid sequence comprising any one of SEQ ID NOs: 40 to 47 or a biologically active fragment or derivative thereof. In some aspects, the engineered nucleic acid construct or engineered expression vector comprises a polynucleotide sequence encoding for a polypeptide having an amino acid sequence comprising any one or more of SEQ ID NOs: 13 to 24 or a biologically active fragment or derivative thereof, or SEQ ID NOs: 40 to 47 or a biologically active fragment or derivative thereof, or any combination thereof.

In various aspects the current disclosure also encompasses engineered nucleic acid constructs or engineered vector comprising a nucleotide sequence encoding for a polypeptide or RNA effector that impacts the expression and/or functioning of the SDP and SKF proteins. These may include but are not limited to chaperones, (for example CsaA), maturation factors (for example signal peptidases like SipS and/or SipT), proteins effectors for posttranslational modifications (for example disulfide bond isomerases like BdbB or BdbC), secretory pathway proteins (for example SecA, SecY, SecE, SecG), proteins involved in cell lysis like Lyt operon proteins, holins, autolysins etc. In some aspects the current disclosure also encompasses nucleic acid constructs and engineered vector comprising a nucleotide sequence that impacts the expression of the SDP/SKF proteins. These may include but are not restricted to enhancers, UTRs.

In various embodiments, the nucleic acid sequence encoding one or more of the cell lysis protein or peptide (e.g., an SKF or SDP protein or fragment or derivative thereof, or a Lyt protein or fragment or derivative thereof) is operably linked to an inducible heterologous promoter system. Non-limiting examples of heterologous promoter systems include LacI repressor system, CtsR repressor system, ManR activator system, T7 promoter system and use of synthetic promoters for highly constitutively active gene expression (e.g., P₄₃, P_{grac}100). The inducible promoter can be chosen so that cell lysis of a cell comprising the engineered nucleic acid construct or expression vector can be carefully controlled.

In various aspects, the inducible promoter system can comprise for example a thermosensitive, a chemosensitive, or a photosensitive regulator (e.g., an activator or repressor). Exemplary thermosensitive promoter systems include P_{clpE}, P_{ctsR}, and a CtsR/P_{clpE} system. Exemplary chemosensitive promoter systems include P_{grac}100, P_{spac}, P_{xylA}, P_{licB}, PT7_{lac}, P_{manP}, P_{manR}, or P_{spac}.

In general, the inducible promoter system can comprise (a) a promoter/operon/transcription factor that controls and facilitates expression of the protein of interest (e.g., the cell lysis protein) and (b) a second promoter/operon/transcription factor/regulator that controls expression of the promoter/transcription factor in (a). As would be understood by one of skill in the art, any number of different combinations of first and second promoters can be used. Further, the system can comprise more than two promoters (e.g., three, four, five, etc. . . . ) each operably linked to ultimately control expression of the cell lysis protein or peptide. The system can be rendered inducible by linking the expression or activity of any of these promoters to a promoter with activity modified by exposure to an external stimulus (e.g., a chemical, heat, light). Alternatively, the system can be rendered inducible by using regulating proteins (like a repressor) and linking the activity of the regulating protein to an external stimulus.

As a non-limiting example, the inducible promoter system can comprise a nucleic acid sequence encoding a promoter operably linked to an inducible repressor. In various aspects, the inducible repressor can be thermosensitive, chemosensitive, or photosensitive. For example, the inducible repressor can be thermosensitive and in various instances can comprise a thermosensitive CtsR repressor. In various aspects, the inducible repressor (e.g., CtsR) can repress expression of the promoter operably linked to the gene(s) encoding the SKF or SDP protein or peptide. For example, in various aspects, the promoter operably linked to the gene(s) encoding the cell lysis protein or peptide can be a clpE promoter that is repressed by CtsR. Accordingly, in one non-limiting example, the inducible promoter system that controls the expression of the cell lysis proteins can comprise a thermosensitive CtsR/clpE system. In this system, an elevated temperature can inactivate the CtsR repressor, and thereby increasing expression of a clpE promoter and the cell lysis proteins. In effect, this system results in the controlled lysis of cells in a cell control by a simple change in temperature—allowing for more productive yield and higher collection of any protein expressed by those cells.

Other inducible promoter systems can be used in place of the CtsR/clpE system described herein. For example, LacI/P_{spac}, XylR/xylA, SigB/SigB, T7 promoter system can all be used.

In various aspects, the inducible promoter system operably linked to the polynucleotide encoding the cell lysis protein or peptide is completely heterologous to the cell lysis protein or peptide as it occurs in nature. In other words, the inducible promoter does not comprise a promoter operably linked to the cell lysis protein or peptide as it occurs in nature (e.g., the Spo0A regulator or component thereof). In other aspects, the inducible promoter can be derived from a promoter or regulator operably linked to the cell lysis protein or peptide as it occurs in nature (e.g., the Spo0A regulator or component thereof) and made inducible by, for example, operably linking it to another inducible promoter or regulator.

In various aspects, altering an endogenous promoter or regulator normally linked to a cell lysis protein or peptide can occur via a “promoter exchange” where a native gene in a recombinant cell (e.g., an endogenous cell lysis gene in a cell line) is engineered to be operably linked to a promoter that controls expression of the cell lysis gene differently than normal expression. For example, the expression of native operon for the SKF/SDP system in Bacillus subtilis is affected by the Spo0A master regulator. In some embodiments, this regulator or another endogenous promoter for a cell lysis gene can be modified, for example disabled, excised (and not replaced) or removed and replaced with an inducible promoter. In various aspects, the inducible promoter may already be native to the cell. For example, as described above, the cell lysis gene can be operably linked to a clpE promoter, which is a native promoter in B. subtilis but it not normally linked to cell lysis protein expression. Accordingly, in various aspects the non-native nucleic acid construct or non-native expression vector can be derived from a chromosomal or genetic sequence in a host cell. In other embodiments, the non-native nucleic acid construct or non-native expression vector can comprise an exogenous vector (e.g., a plasmid, cosmid, or other construct or vector) that can exist separately from a chromosomal or genomic DNA of a host cell but can be delivered to a cell. Accordingly, in various embodiments, the polynucleotide encoding the cell lysis protein or peptide is endogenous to the expression system. In other embodiments, the polynucleotide encoding the cell lysis protein or peptide is exogenous to the expression system.

(iv) Engineered Heterologous Gene Expression Vectors and Constructs

Bacillus subtilis is considered as a universal cell factory or production system for industry, agriculture and biomaterial applications including chemicals, enzymes and antimicrobials. One aspect of the current disclosure is to develop constructs and systems to improve production and harvest of materials from Bacillus subtilis and related strains.

In some aspects, the current disclosure encompasses the engineered nucleic acid construct or engineered expression vector and cell lysis systems that maximize expression, production and/or recovery of heterologous polypeptides in Bacillus subtilis and related strains. Accordingly, in various aspects, the engineered nucleic acid construct or engineered expression vector for heterologous expression may comprise a nucleotide sequence encoding a polypeptide for one or more heterologous polypeptides as provided in section A(ii). In some aspects the nucleotide sequence encoding the polypeptide may be codon optimized for protein expression in Bacillus.

In an exemplary aspect, the engineered nucleic acid construct or engineered expression vector encodes a αS1-Casein. Accordingly, the engineered nucleic acid construct or engineered expression vector comprises a nucleotide sequence having at least 85%, at least 90%, at least 95%, or at least 99% sequence identity to SEQ ID NO: 25. In an aspect, the engineered nucleic acid construct or engineered expression vector comprises a nucleotide sequence encoding for a polypeptide having at least 85%, at least 90%, at least 95%, or at least 99% sequence identity to SEQ ID NOS: 26. In various aspects, the engineered nucleic acid construct or engineered expression vector comprises a polynucleotide sequence encoding for a polypeptide having an amino acid sequence comprising SEQ ID NO: 25 or 26.

In various other exemplary aspects, the engineered nucleic acid construct or engineered expression vector for heterologous expression may comprise a nucleotide sequence encoding a polypeptide for one or more heterologous polypeptides of relevance to the application for which the heterologous expression is desired.

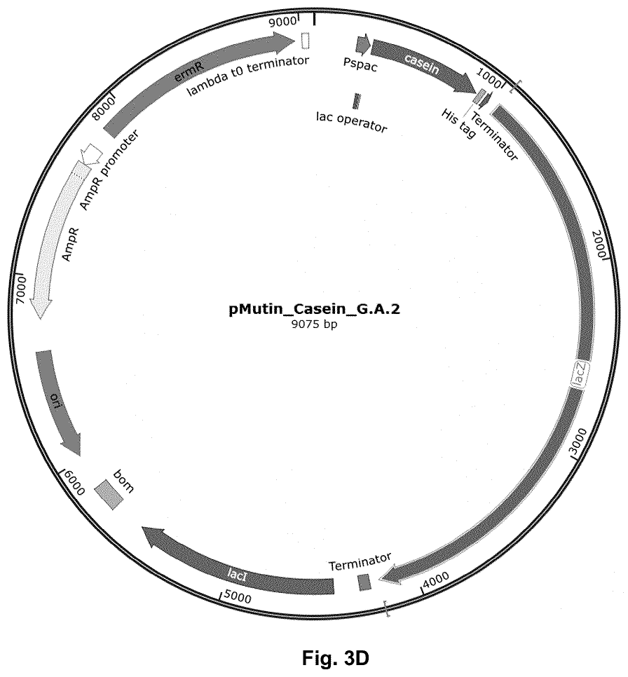

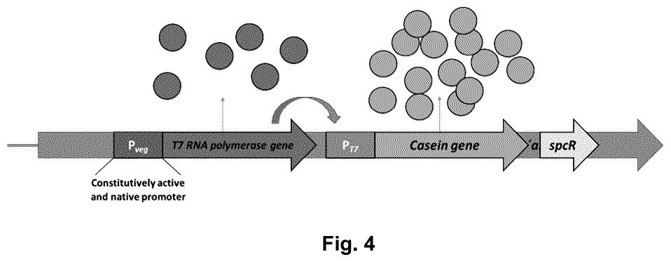

In various aspects, a polynucleotide encoding for the heterologous polypeptide can be operably linked to a separate promoter system. In general, the separate promoter system is native or synthetic to facilitate high expression of the heterologous polypeptide in a recombinant cell line (e.g., the host cell described above). In some aspects, the promoter system may be a constitutive promoter system. In some aspects the promoter system maybe an inducible promoter system. In some aspect the promoter may be a native Bacillus promoter. In some aspects the promoter system may be heterologous to Bacillus. In some aspects, the second promoter system facilitates the expression of a regulator or transcription factor that, in turn, controls and maintains strong expression of the heterologous polypeptide. A second/separate promoter system can be, for example, a T7 promoter system, where expression of a T7 RNA polymerase is linked to a constitutive or inducible promoter. In various aspects, the constitutive promoter is native or endogenous to a recombinant cell line expressing the heterologous polypeptide. In various aspects, the constitutive promoter is non-native to a recombinant cell line expressing the heterologous polypeptide. Exemplary second promoters that are contemplated for use in various aspects of this disclosure include a native or synthetic promotor for expression of the T7-RNA-polymerase gene. As a non-limiting example, FIG. 4 shows an exemplary system linking an endogenous P veg promoter with T7 promoter expression to drive continuous expression of a heterologous polypeptide (casein) in accordance with various aspects of this disclosure.

In various aspects, the second inducible promoter system comprises a nucleic acid encoding a heterologous polypeptide operably linked to an inducible promoter. Suitable inducible promoters used in this second inducible system are ideally distinct from the inducible promoter used to control expression of the cell lysis proteins or peptides. Here, they are induced in a different way from the promoter of the cell lysis proteins or peptides. Inducible promoter system can comprise for example a thermosensitive, a chemosensitive, or a photosensitive regulator (e.g., an activator or repressor). Exemplary thermosensitive promoter systems include CtsR regulator and the promoter region upstream of the clpE gene. Exemplary chemosensitive promoter systems include P_{grac}100, P_{spac}, P_{xylA}, P_{licB}, PT7_{lac}, P_{manP}, P_{manR}.

(v) Engineered Cell Lysis and Expression System

An engineered cell lysis system as contemplated herein includes at least an engineered expression nucleic acid construct comprising a nucleic acid sequence encoding at least one cell lysis protein or a derivative or fragment thereof, operably linked to an inducible promoter system for controlling the expression of the cell lysis protein or peptide. It will be understood that an engineered cell lysis system may include multiple other elements relating to expression of the heterologous polypeptide, and that the elements may be assembled and introduced to the host cell in various ways, such as in one or more vectors, as described further below. Accordingly, any of the engineered nucleic acid constructs or engineered expression vectors described above in section (a) may be incorporated into the engineered cell lysis system. As described further below, the nucleic acid constructs and/or expression vectors can be localized to a cell (e.g., a host cell) which may, optionally, include other constructs or vectors to facilitate expression of other proteins or peptides of interest (e.g., a commercially important heterologous polypeptide).

In various aspects, the engineered cell lysis system comprises a first nucleic acid construct comprising a nucleotide sequence encoding a cell lysis promoting protein or peptide operably linked to an inducible heterologous promoter system (e.g., an engineered nucleic acid construct provided above), and optionally further comprises a second nucleic acid construct comprising a polynucleotide encoding a heterologous polypeptide operably linked to a separate promoter system. The first and second nucleic acid constructs may be provided as one or more expression vectors as described below.

One or more expression vectors are provided having a nucleotide sequence encoding one or more components of the engineered cell lysis system provided herein. In various aspects, an expression vector (e.g., as described in section (a)) can comprise a polynucleotide encoding the cell lysis proteins or peptides operably linked to an inducible promoter system. In some aspects, a single expression vector is provided having (a) a polynucleotide sequence encoding the cell lysis promoting protein operably linked to an inducible heterologous promoter system and (b) a polynucleotide sequence encoding a heterologous polypeptide operably linked to a separate promoter system. In various aspects, a pair of dual vectors is provided, wherein a first expression vector sequence comprises the nucleic acid sequence of (a) and a second expression vector sequence comprising the nucleic acid sequence of (b).

Any of the expression vectors provided herein may comprise additional expression control sequences (e.g., enhancer sequences, Kozak sequences, polyadenylation sequences, transcriptional termination sequences, etc.), selectable reporter sequences (e.g., antibiotic resistance genes), origins of replication, T-DNA border sequences, and the like. The plasm id or viral vector may further comprise RNA processing elements such as glycine tRNAs, or Csy4 recognition sites. Additional information about vectors and use thereof may be found in “Current Protocols in Molecular Biology”, Ausubel et al., John Wiley & Sons, New York, 2003, or “Molecular Cloning: A Laboratory Manual”, Sambrook & Russell, Cold Spring Harbor Press, Cold Spring Harbor, NY, 3rd edition, 2001.

Likewise, nucleic acid constructs provided herein may be DNA or RNA, linear or circular, single-stranded or double-stranded, or any combination thereof. The nucleic acid constructs may be codon optimized for efficient translation into protein in the cell of interest. Codon optimization programs are available as freeware or from commercial sources.

In various aspects, the inducible cell lysis system is incorporated or delivered into a host cell. Therefore, a host cell is provided comprising the engineered nucleic acid construct or engineered expression vector that allows for inducible expression of a cell lysis protein or peptide, as described above.

In some aspects, the engineered cell lysis system can comprise a modified chromosomal or genomic polynucleotide of the host cell. For example, as described above, various host cells (e.g., Bacillus subtilis) endogenously comprise polynucleotide sequences for cell lysis proteins or peptides (e.g., the SDP/SKP system, LYT system, holin-like proteins (for example BhlA, BhlB), autolysins like CwlA, or fragments or derivatives thereof and any combination thereof). In various aspects, the engineered cell lysis system can comprise an endogenous polynucleotide encoding for a cell lysis protein or peptide operably linked to a heterologous inducible promoter system. Likewise, the second nucleic acid construct comprising a polynucleotide encoding the heterologous polypeptide, if present, can be incorporated into a chromosomal or genomic polynucleotide.

II. Host Cells/Recombinant Cell Lines

The present disclosure also provides a host cell comprising any non-native nucleic acid construct or expression vector, and/or an engineered cell lysis and expression system, as described herein.

In various aspects, the host cell can be from a cell line comprising an endogenous cell lysis protein or peptide gene. In various aspects, the host cell can comprise a cell line where endogenous expression of the cell lysis protein or peptide is modified. In some aspects, the host cell may be a prokaryotic cell. In some aspects, the host cell can be a bacterial cell such as, for example, a Bacillus subtilis cell or other sporulating cell. In one non-limiting example, the host cell is a bacterial cell comprising a deletion in an immunity SDP or SKF protein and/or a deletion of master regulator for sporulation initiation (e.g., Spo0A) and/or in a deletion of AbrB. In various aspects the host cell may exhibit disrupted or modified expression of one or more genes that negatively impact cell lysis. As used herein the terms “disruption,” “disrupting,” or “disrupted”, “modification” or “modified” refer to genetic modifications that alter/reduce the level of expression of a target gene. In some aspects, the disruption can be due to a deletion of at least one nucleotide within or near the target gene or a deletion of part or all of a target gene, as described above. In other aspects, the disruption also can be due to a substitution of at least one nucleotide and/or an insertion of at least one nucleotide within or near the target gene. In further aspects, the disruption can be due to an insertion of one or more exogenous polynucleotides within or near the target gene. In some aspects the reduced or lack of expression could be due to a modification in the promoter, enhancer, regulatory sequence and in genes affecting the expression of these gene. In general, as used herein, modified expression refers to reduced or eliminated expression or induced expression of the target gene. In some embodiments, the modification can result in reduced level of expression (e.g., express less than 30%, less than 25%, less than 20%, less than 10%, less than 5% of the level of an unmodified cell). In some embodiments, the modification can result in eliminated expression (e.g., no expression or an undetectable level of RNA and/or protein expression). Expression can be measured using any standard RNA-based, protein-based, and/or antibody-based detection method (e.g., RT-PCR, ELISA, flow cytometry, immunocytochemistry, and the like). Detectable levels are defined as being higher that the limit of detection (LOD), which is the lowest concentration that can be measured (detected) with statistical significance by means of a given detection method. In some embodiments, the disruption can be an altered expression pattern of one or more gene. In some aspects the expression is temporally altered and inducible. In some exemplary aspects, the expression may be altered by a promoter exchange in the host cell such that the native promoter is replaced by an inducible promoter.

In some aspects the host cell may exhibit modified expression of one or more proteases that impact heterologous gene expression, for example by degrading the protein produced. In some aspects the host cell may be a bacterial cell in which expression of ClpP is modified. In some aspects the host cell is a Bacillus cell, such as a Bacillus subtilis cell, which exhibits modified expression of ClpP.

In some aspects, the host cell may overexpress on or more proteins that promote cell lysis. Non-limiting examples of such genes include SkfA, SkfB, SkfC, SkfE, SkfF, SkfG, SkfH, SdpA, SdpB, SdpC, SdpI, SdpR, SipS, SipT LytA, LytB, LytC, LytE, BlyA, BhlA, BhlB, CwlA.

Table B provides a list of some exemplary genes that may be modified or overexpressed in the host cell of the engineered lysis system.

TABLE C
Engineered host cells

| Overexpression | Modification/deletion |
|---|---|
| sdpA | sdpI |
| sdpB | sdpR |
| sdpC | sigW |
| skfA | yfhL |
| skfB | yknW |
| skfC | yknX |
| skfE | yknY |
| skfF | yknZ |
| skfG | clpP |
| skfH | abrB |
| sipS | |
| sipT | |
| lytA | |
| lytB | |
| lytC | |
| lytE | |
| blyA | |
| bhlA | |
| bhlB | |
| cwlA | |

By way of non-limiting example, the host cell can be, or can be derived from the Bacillus subtilis 3NA cell line. B. subtilis 3NA is a mutant strain of Bacillus subtilis that does not sporulate (due to an inactivated/disabled Spo0A regulator). The complete genome sequence of B. subtilis 3NA can be found in Genome Announc 3(2):e00084-15. and is incorporated herein by reference in its entirety. The present disclosure contemplates that further genetic modifications to B. subtilis 3NA, which are capable of achieving high cell density fermentation, can allow for greater and precise control of cell lysis even under high cell density conditions. Additional non-limiting examples of suitable Bacillus subtilis strains, in the context of which one or more lysis promoting modifications can be contemplated include Bacillus subtilis BMV9 (Δspo0A; abrB*; ΔmanPA; trp⁺), Bacillus subtilis 168 and a reduced genome version of Bacillus subtilis 168 (IIG-Bs-20-1).

Other exemplary prokaryotic cell lines include sporulating bacteria such as, by way of non-limiting example, B. cereus, B. megaterium, B. velezensis, Lysinibacillus, Anoxybacillus, and Paenibacillus, and cell lines derived therefrom, including those comprising a deletion in an immunity SDP or SKF protein (e.g., SdpR/SdpI) and/or a deletion of master regulator for sporulation initiation such as that described for B. subtilis 3NA.

A host cell comprising the engineered cell lysis system may comprise one or more nucleic acid constructs, each including one or more nucleic acid sequences encoding elements of the engineered cell lysis and expression system. In various aspects, the host cell can comprise a polynucleotide encoding the cell lysis proteins or peptides operably linked to an inducible promoter system. In some aspects, the host cell comprises a single expression vector having (a) a polynucleotide sequence encoding the cell lysis promoting protein operably linked to an inducible heterologous promoter system and (b) a polynucleotide sequence encoding a heterologous polypeptide operably linked to a separate promoter system. In various aspects, the host cell comprises a pair of dual vectors, wherein a first expression vector sequence comprises the nucleic acid sequence of (a) and a second expression vector sequence comprising the nucleic acid sequence of (b). In some aspects the host cell may comprise one or more elements of the cell lysis and expression system chromosomally integrated into the host cell.

In various aspects, the host cells are maintained for a time and under conditions sufficient for the protein of interest to be expressed at least to detectable levels, such that the host cells and protein of interest are present in culture together. Methods, appropriate cell media and culture systems for culturing cells for protein expression are well known and readily commercially available. In a non-limiting example, cells can be grown under rotation (e.g., between 120-180 rpm) in a mineral salt medium (e.g., as in Klausmann et al., 2021) or in LB medium using a temperature optimized for cell growth and target protein accumulation (e.g., around 37° C.). If a thermosensitive promoter is linked to the cell lysis proteins, lysis of the cells can be induced between 45 and 52° C. Additional exemplary methods for culturing cells (e.g., in a bioreactor) are described in Klausmann et al., 2021 (“Bacillus subtilis High Cell Density Fermentation Using a Sporulation-Deficient Strain for the Production of Surfactin” Applied Microbiology and Biotechnology (2021) 105: 4141-4151), which is incorporated herein by reference in its entirety.

III. Methods for Generating Non-Native Nucleotide Constructs, Expression Vectors and Cell Lines

Recombinant production of a protein in a host cell, e.g., a bacterial cell, may provide for a more desirable vehicle for producing the protein in commercially relevant quantities. The recombinant production of a protein is generally accomplished by constructing an expression cassette in which the DNA coding for the protein is placed under the expression control of a promoter from a regulated gene. The expression cassette is introduced into the host cell, usually by plasmid-mediated transformation, or phage, viral or transposon transduction. Production of the protein is then achieved by culturing the transformed host cell under inducing conditions necessary for the proper functioning of the promoter contained on the expression cassette.

Fungal cells may be transformed with a vector by a process involving protoplast formation, transformation of the protoplasts, and regeneration of the cell wall in a manner known per se. Transformation of a fungal host cell with two or more vectors, alone or together (co-transformation) is very inefficient and limited by the availability of useful selectable markers. The engineered nucleic acid or nucleotide constructs, and/or the expression vectors described herein can be produced using standard recombinant methods.

Those of skill in the art will also appreciate that expression vectors as described herein can comprise additional regulatory sequences (e.g., termination sequence, translational control sequence, etc.), as well selectable marker sequences. Plasm ids are known in the art, including those based on pBR322, PUC, and so forth. Viral vectors may also be used to provide intracellular expression of the cell lysis protein or peptide. Suitable viral vectors include bacteriophages, retroviral vectors, lentiviral vectors, adenoviral vectors, adeno-associated virus vectors, herpes virus vectors, and so forth.

The recombinant cell lines expressing the cell lysis system described herein can be produced using standard methods as well. For example, in B. subtilis, natural competence development and electroporation can be used for transformation of genetic material. In various aspects, plasm ids can be constructed using Gibson Assembly with any required restriction enzymes, alkaline phosphatases and ligases as understood by one of skill in the art. In additional aspects, Phusion PCR can be used to fuse different DNA segments. Chromosomal integration can be achieved by using homologous recombination of target sequences, using, for example a gene editing technology such as a CRISPR/Cas system, a zinc finger nuclease, or a Transcription activator-like effector nuclease (TALEN).

IV. Methods

A further aspect of the present disclosure provides a method for making and recovering a heterologous polypeptide produced by a cell in culture. The method can comprise (a) introducing or having introduced a cell lysis protein or peptide to the cell, wherein the cell expresses the heterologous polypeptide; (b) inducing expression of the cell lysis protein or peptide in the cell, thereby lysing the cell to form a lysed cell mixture; and (c) harvesting the heterologous polypeptide from the lysed cell mixture. Some other aspects the method can comprise (a) introducing or having introduced a heterologous polypeptide expression system, (b) introducing or having introduced a cell lysis protein or peptide to the cell, (c) inducing the expression of the heterologous polypeptide; (b) inducing expression of the cell lysis protein or peptide in the cell, thereby lysing the cell to form a lysed cell mixture; and (c) harvesting the heterologous polypeptide from the lysed cell mixture

In further aspects, a method for recovering a heterologous polypeptide produced in a cell culture is provided. In various aspects, the method comprises (a) culturing a host cell provided herein comprising the inducible cell lysis system, under conditions sufficient for expression of the heterologous polypeptide; (b) inducing the expression of the cell lysis protein or peptide thereby lysing the cell to form a lysed cell mixture; and (c) harvesting the heterologous polypeptide from the lysed cell mixture.

As used herein, the term “culturing” refers to any method that can be used for proliferating the cells of the current disclosure. Methods of culturing cells, for example Bacillus subtilis cells are well known in the art and can be suitably modified to enhance the expression, and/or subsequent lysis and/or recovery of the heterologous polypeptide. In some aspects, the cells can be cultured in a small-scale culture system, for example a flask, or a mid to large scale culture system for example a fermentation system. Fermentation methods well known in the art can be applied to ferment the modified and unmodified Bacillus cells of the disclosure.

In some aspects, the cells are cultured under batch or continuous fermentation conditions. A classical batch fermentation is a closed system, where the composition of the medium is set at the beginning of the fermentation and is not altered during the fermentation. At the beginning of the fermentation, the medium is inoculated with the desired organism(s) for example a engineered host cell of the current disclosure. In this method, fermentation is permitted to occur without the addition of any components to the system. Typically, a batch fermentation qualifies as a “batch” with respect to the addition of the carbon source, and attempts are often made to control factors such as pH and oxygen concentration. The metabolite and biomass compositions of the batch system change constantly up to the time the fermentation is stopped. Within typical batch cultures, cells can progress through a static lag phase to a high growth log phase, and finally to a stationary phase, where growth rate is diminished or halted. If untreated, cells in the stationary phase eventually die. In general, cells in log phase are responsible for the bulk of production of product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO2. Batch and fed-batch fermentations are common and known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one aspect, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being drawn off should be balanced against the cell growth rate in the fermentation. Methods of modulating nutrients and growth factors for continuous fermentation processes, as well as techniques for maximizing the rate of product formation, are well known in the art of industrial microbiology.

Thus, in certain aspects, a heterologous polypeptide produced by a transformed/transduced or modified host cell may be recovered from the culture medium by conventional procedures including separating the host cells or cell debris from the medium by centrifugation or filtration, or if necessary, induction of cell lysis where suitable and removing the supernatant from the cellular fraction and debris. Typically, after clarification, the proteinaceous components of the supernatant or filtrate are precipitated by means of a salt, e.g., ammonium sulfate. The precipitated proteins are then solubilized and may be purified by a variety of chromatographic procedures, e.g., ion exchange chromatography, gel filtration.

In various aspects, inducing expression of the cell lysis protein or peptide and/or heterologous polypeptide comprises applying one or more external stimuli to the cell or cell culture. In various aspects, the external stimulus can be heat or light (e.g., when expression of the cell lysis protein or peptide is controlled by a heat sensitive protein). In other aspects, the external stimulus can be a chemical or a nutrient (e.g., when expression of the cell lysis protein or peptide is controlled by a chemical or nutrient dependent protein). In various aspects, applying an external stimulus can also comprise depriving the culture or cells of a necessary nutrient or chemical.

In some aspects, the current disclosure also encompasses methods for enhancing the yield or recovery or both, of the one or more heterologous polypeptides of interest. In some aspects, the disclosed methods can reduce the cost of production and harvesting of the heterologous polypeptide of interest. In some aspects, the disclosed methods can help increase the yield of the heterologous polypeptide of interest. In some aspects, the yield may be improved by at least about 0.1%, at least about 0.5%, at least about 1%, at least about 5%, at least about 6%, at least about 7%, at least about 8%, at least about 9%, or at least about 10% or more of the heterologous polypeptide of interest, relative to an unmodified parental or wild type cell.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

A “genetically modified” cell refers to a cell in which the nuclear, organellar or extrachromosomal nucleic acid sequences of a cell has been modified, i.e., the cell contains at least one nucleic acid sequence that has been engineered to contain an insertion of at least one nucleotide, a deletion of at least one nucleotide, and/or a substitution of at least one nucleotide.

The terms “genome modification” and “genome editing” refer to processes by which a specific nucleic acid sequence in a genome is changed such that the nucleic acid sequence is modified. The nucleic acid sequence may be modified to comprise an insertion of at least one nucleotide, a deletion of at least one nucleotide, and/or a substitution of at least one nucleotide. The modified nucleic acid sequence is inactivated such that no product is made. Alternatively, the nucleic acid sequence may be modified such that an altered product is made.

The term “heterologous” refers to an entity that is not native to the cell or species of interest.

The terms “nucleic acid” and “polynucleotide” refer to a deoxyribonucleotide or ribonucleotide polymer, in linear or circular conformation. For the purposes of the present disclosure, these terms are not to be construed as limiting with respect to the length of a polymer. The terms may encompass known analogs of natural nucleotides, as well as nucleotides that are modified in the base, sugar and/or phosphate moieties. In general, an analog of a particular nucleotide has the same base-pairing specificity, i.e., an analog of A will base-pair with T. The nucleotides of a nucleic acid or polynucleotide may be linked by phosphodiester, phosphothioate, phosphoramidite, phosphorodiamidate bonds, or combinations thereof.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or an engineeredly occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

A “wild type” protein amino acid sequence can refer to a sequence that is naturally occurring and encoded by a germ line genome. A species can have one wild type sequence, or two or more wild type sequences (for example, with one canonical wild type sequence and one or more non-canonical wild type sequences). A wild type protein amino acid sequence can be a mature form of a protein that has been processed to remove N-terminal and/or C-terminal residues, for example, to remove a signal peptide. An amino acid sequence that is “derived from” a wild type sequence or other amino acid sequence disclosed herein can refer to an amino acid sequence that differs by one or more amino acids compared to the reference amino acid sequence, for example, containing one or more amino acid insertions, deletions, or substitutions as disclosed herein. The terms “derivative,” “variant,” “variations” and “fragment,” when used herein with reference to a polypeptide, refers to a polypeptide related to a wild type polypeptide, for example either by amino acid sequence, structure (e.g., secondary and/or tertiary), activity (e.g., enzymatic activity) and/or function. Derivatives, variants, “variations” and fragments of a polypeptide can comprise one or more amino acid variations (e.g., mutations, insertions, and deletions), truncations, modifications, or combinations thereof compared to a wild type polypeptide. A part or fragment of a polypeptide may correspond to at least 1%, at least 2%, at least 3%, at least 4%, at least 5%, at least 10%, at least 20%, at least 30%, at least 40% of the length of a polypeptide, such as a polypeptide having an amino acid sequence identified by a specific SEQ ID NO., or having at least 50%, or at least 60%, or at least 70%, or at least 80%, or at least 90% of the length (in amino acids) of the polypeptide.

Within the context of the present application, a protein is represented by an amino acid sequence, and correspondingly a nucleic acid molecule or a polynucleotide is represented by a nucleic acid sequence. Identity and similarity between sequences: throughout this application, it should be understood that for each reference to a specific amino acid sequence using a unique sequence identifier (SEQ ID NO.), the sequence may be replaced by: a polypeptide represented by an amino acid sequence comprising a sequence that has at least 60% sequence identity or similarity with the reference amino acid sequence. Another preferred level of sequence identity or similarity is 65%. Another preferred level of sequence identity or similarity is 70%. Another preferred level of sequence identity or similarity is 75%. Another preferred level of sequence identity or similarity is 80%. Another preferred level of sequence identity or similarity is 85%. Another preferred level of sequence identity or similarity is 90%. Another preferred level of sequence identity or similarity is 95%. Another preferred level of sequence identity or similarity is 98%. Another preferred level of sequence identity or similarity is 99%.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length (in amino acids or nucleotides) of two given SEQ ID NOS or based on a portion thereof. A portion of a full-length sequence may be referred to as a fragment, and preferably means at least 50%, 60%, 70%, 80%, 90%, or 100% of the length (in amino acids or nucleotides) of a reference sequence. “Identity” also refers to the degree of sequence relatedness between two amino acid sequences, or between two nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [b162], Gap Open: 1.53, Gap extension: 0.123, Order: aligned, Tree rebuilding number: 2, Guide tree output: ON [true], Max iterate: 2, Perform FFTS: none is used).

As used herein, the terms “target site”, “target sequence”, or “nucleic acid locus” refer to a nucleic acid sequence that defines a portion of a nucleic acid sequence to be modified or edited or disrupted and to which a homologous recombination composition may be engineered to target.

The terms “upstream” and “downstream” refer to locations in a nucleic acid sequence relative to a fixed position. Upstream refers to the region that is 5′ (i.e., near the 5′ end of the strand) to the position, and downstream refers to the region that is 3′ (i.e., near the 3′ end of the strand) to the position.

A protein of interest (POI) of the instant disclosure can be any endogenous or heterologous polypeptide, and it may be a variant, derivative or fragment of such a POI. The protein can contain one or more disulfide bridges or is a protein whose functional form is a monomer or a multimer, i.e., the protein has a quaternary structure and is composed of a plurality of identical (homologous) or non-identical (heterologous) subunits, wherein the POI or a variant POI thereof is preferably one with properties of interest.

Techniques for determining nucleic acid and amino acid sequence identity are known in the art. Typically, such techniques include determining the nucleotide sequence of the mRNA for a gene and/or determining the amino acid sequence encoded thereby and comparing these sequences to a second nucleotide or amino acid sequence. Genomic sequences may also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=H IGH SCORE; Databases=non-redundant, GenBank+EMBL+DDBJ+PDB+GenBank CDS translations+Swiss protein+Spupdate+PIR. Details of these programs may be found on the GenBank website. With respect to sequences described herein, the range of desired degrees of sequence identity is approximately 80% to 100% and any integer value therebetween. Percent identities between sequences can be at least 70-75%, at least 80-82%, at least 85-90%, at least 92%, at least 95%, or at least 98% sequence identity.

As used herein the term “cell lysis promoting polypeptide” or “cell lysis promoting protein” or “cell lysis promoting peptide” corresponds to any polypeptide sequence which by its presence in a cell or overexpression in a cell, directly or indirectly (i.e., by its' influence on another cell factor such as a protein, polypeptide, peptide, polynucleotide including DNA or RNA, RNA and/or protein expression, RNA and/or protein degradation) enhances the lysis of a cell for example Bacillus subtilis, when compared to another cell which does not comprise or overexpress said polypeptide.

As various changes could be made in the above-described cells and methods without departing from the scope of the invention, it is intended that all matter contained in the above description and in the examples given below, shall be interpreted as illustrative and not in a limiting sense.

### EXAMPLES

All patents and publications mentioned in the specification are indicative of the levels of those skilled in the art to which the present disclosure pertains. All patents and publications are herein incorporated by reference to the same extent as if each individual publication was specifically and individually indicated to be incorporated by reference.

The publications discussed throughout are provided solely for their disclosure before the filing date of the present application. Nothing herein is to be construed as an admission that the invention is not entitled to antedate such disclosure by virtue of prior invention.

The following examples are included to demonstrate the disclosure. It should be appreciated by those of skill in the art that the techniques disclosed in the following examples represent techniques discovered by the inventors to function well in the practice of the disclosure. Those of skill in the art should, however, in light of the present disclosure, appreciate that many changes could be made in the disclosure and still obtain a like or similar result without departing from the spirit and scope of the disclosure, therefore all matter set forth is to be interpreted as illustrative and not in a limiting sense.

Bacillus subtilis is considered as a universal cell factory or production system for industry, agriculture and biomaterial applications including chemicals, enzymes and antimicrobials. One aspect of the current disclosure is to develop constructs and systems to improve production and harvest of materials from Bacillus subtilis and related strains.

The following examples explore the use of B. subtilis BMV9 (Δspo0A; abrB*; ΔmanPA; trp⁺) strain, and multiple originally constructed derivative strains for heterologous polypeptide production. Strain BMV9 has a nonsense mutation in spo0A which makes it a non-sporulating strain particularly useful for high cell density fermentation processes. In addition, the elongation of abrB gene that is present in the parent 3NA strain confers natural competence to BMV9, making it easier to modify the strains. Importantly, the strains were engineered and tested for inducible self-lysis capabilities for ease of harvest at large scale. αS1-Casein production was tested in these studies. However, production of other test proteins selected from β-casein, k-casein, bovine hemoglobin, bovine myoglobin, seal myoglobin, fish myoglobin, soy leghemoglobin, lactoglobulin, ovalbumin, or α-lactalbumin may be tested.

### Example 1. Production of Bovine αS1 Casein by Bacillus subtilis

Production of Casein in Bacillus subtilis was tested in this exemplary case. For this purpose, bovine αS1 casein was codon-optimized with regard to B. subtilis codon usage (SEQ ID NO: 25).

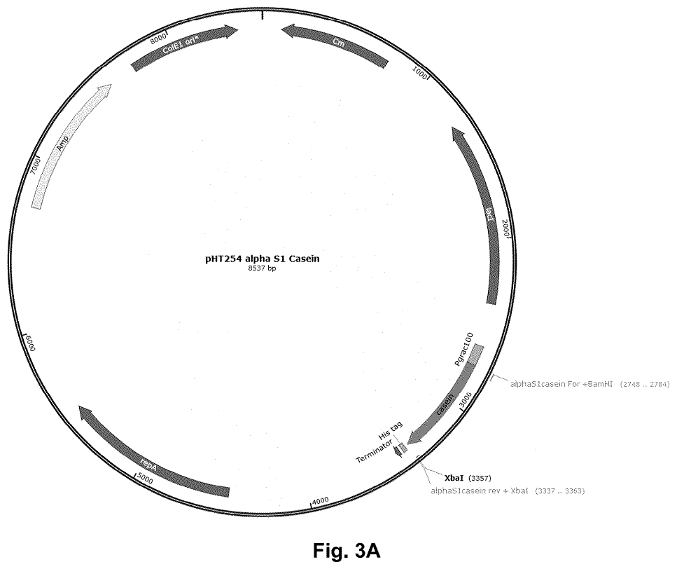

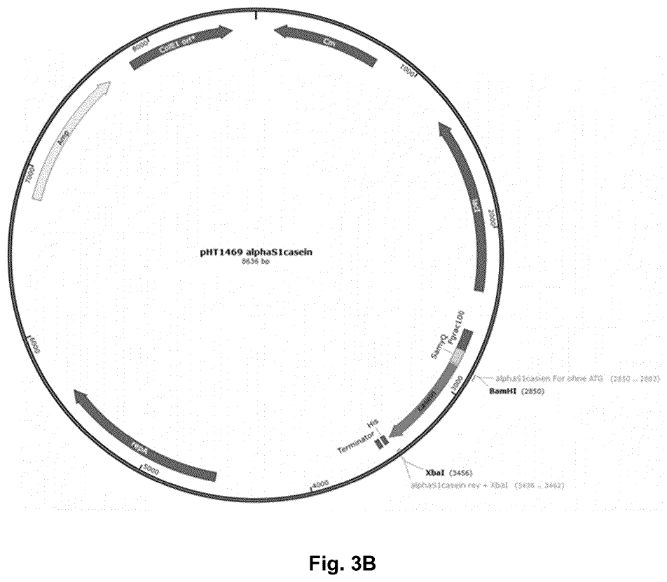

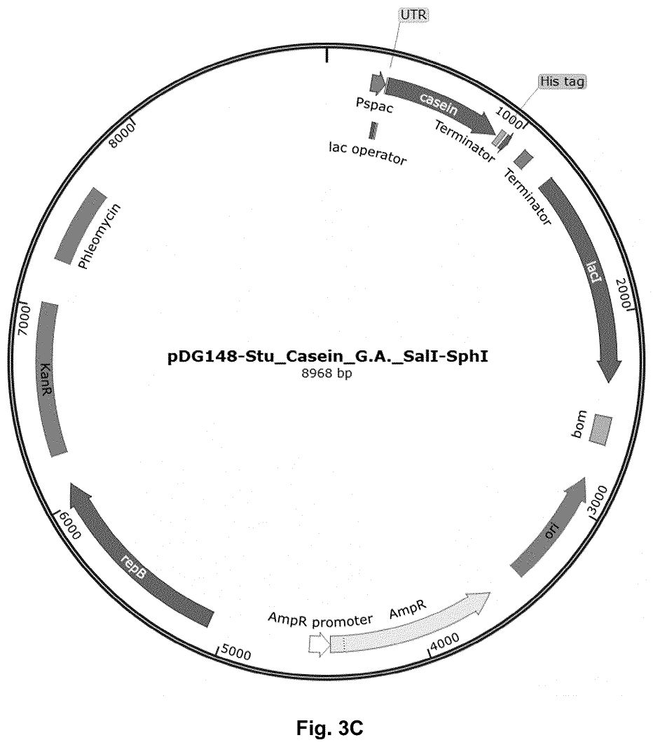

To ensure the highest possible αS1 casein production and accumulation prior to lysis, different shuttle vectors expression systems were tested. This included standard expression constructs including pHT254 and pHT1469 that use the P_{grac}100 promoter for gene expression and cause the expressed protein to form inclusion bodies. Additionally, IPTG inducible Pspac promoter expression systems including pDG148-Stu and pMutin were also tested. Both later tested vectors allow rapid cloning with high translational efficiency. FIGS. 3A-D provides the plasmid map of pHT254 (Phan et al. Novel plasmid-based expression vectors for intra- and extracellular production of recombinant proteins in Bacillus subtilis, pHT1469, pDG148-Stu and pMutin respectively with the αS1 casein gene.

Additionally, a chromosomal integration of a codon optimized αS1 casein (SEQ ID NO: 25), under a T7 RNA polymerase system was also tested. For this, a polynucleotide encoding the T7 RNA polymerase from Escherichia phage T7 was codon-optimized for B. subtilis and integrated at the amyE locus of the chromosome of strain BMV9. Different native promoter sequences from B. subtilis were tested for an optimal expression of T7 RNA polymerase. In addition, different altered T7 promoter regions were coupled with the αS1 casein gene. Finally, different promoter combinations (for T7 RNA polymerase and αS1 casein expression) were tested for the most promising αS1 casein expression and accumulation. FIG. 4 provides a schematic of the integrated construct.

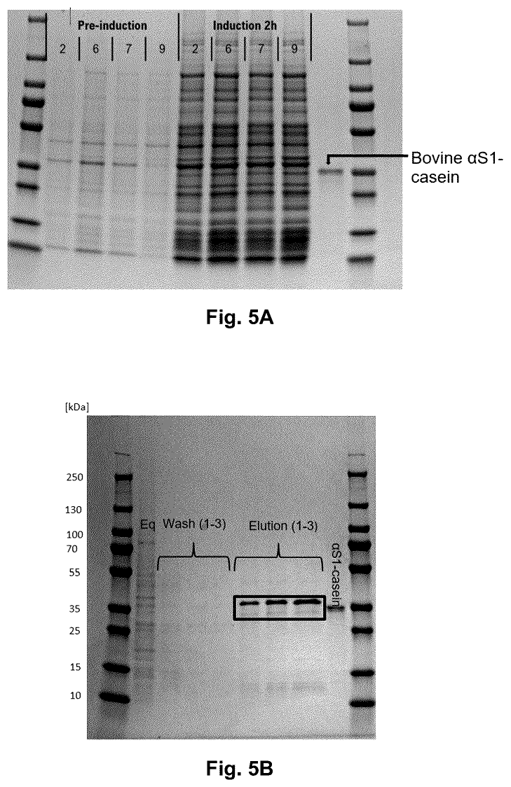

Due to the hydrophobic nature of αS1 casein, the expressed protein tends to accumulate in B. subtilis inclusion bodies which were isolated and analysed using standard protocols. Of the tested systems, both pHT254 (FIG. 5A) and pDG148-Stu expression systems provided robust expression of αS1 casein and the protein expressed from pDG148-Stu could be purified using a c-terminal 6his-tag using the Ni-NTA purification system (see FIG. 5B).

### Example 2: Programmed Cell Lysis System

After intracellular αS1 casein accumulation in inclusion bodies in a bacterial host cell, an economical way of cell disruption is necessary. B. subtilis encodes the operons skfABCEFGH and sdpABC, which facilitate selective lysis of cells when resources are limited, to delay the sporulation process in surviving cells. Both the skf and sdp operons are under control of the master regulator Spo0A which also facilitates the expression of immunity factors—ensuring that sporulating cells are spared from cell lysis. This system makes intracellular nutrients available for sporulating cells so that survival is ensured. Accordingly, under native conditions the Skf and Sdp killing factors operate for the cannibalism of B. subtilis cells, to the survival benefit of the species. The system can however be advantageously engineered for programmable cell lysis.

a) Strain Selection

As a first step towards engineering a production strain B. subtilis BMV9 strain, which is a derivative of a sporulation deficient 3NA strain was selected as a starting point. The strain has a nonsense mutation in the spo0A gene, which renders this master regulator gene non-functional for sporulation initiation. Thus, the 3NA and the derivative BMV9 production strains are sporulation-deficient B. subtilis. Being sporulation deficient, when expression of the Skf and Sdp killing factors is induced in a cell suspension of modified B. subtilis BMV9, without inducing expression of the genes that are the immunity factors, the entire cell suspension may be subject to complete cell lysis. However, to further ensure that the native skf and sdp genes were not expressed and expression could only be induced after a desired level of protein production, the native chromosomal skf and sdp clusters needed to be replaced by an inducible version. This could be achieved by either deleting the native skf and sdp gene clusters and expressing the relevant toxins from a construct integrated at the locus or elsewhere in the chromosome or from a plasmid. The system has the advantage of being programmable as any selection of proteins (including but not restricted to one or more of the Sdp or Skf) can be expressed in a tightly regulated manner. Alternatively, a promoter exchange with an inducible non-native promoter can be done to just express the toxin gene clusters.

b) Deletion of One or More Skf and Sdp Cluster Genes from B. subtilis BMV9

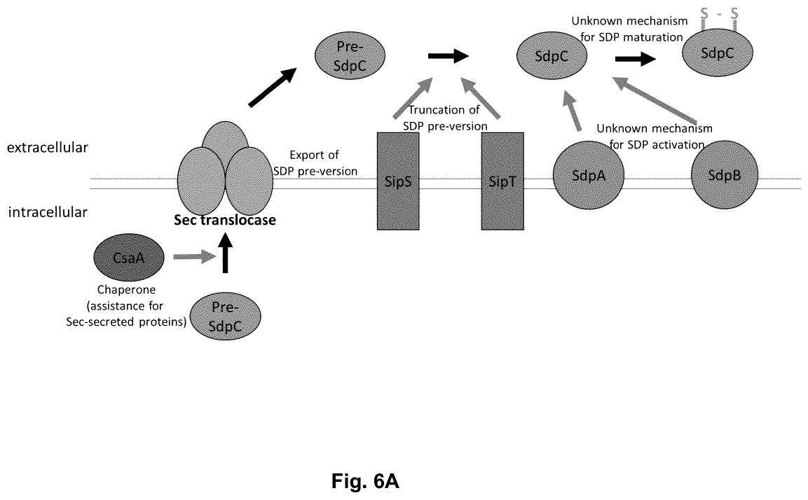

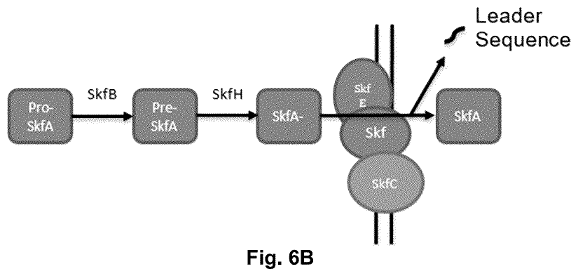

Multiple strains were constructed (see Table 1, FIGS. 6A and 6B) with deletion in the skf and sdp gene clusters using homologous recombination. Deletion was conducted essentially as in Warth L and Altenbuchner J. 2013 using the chimeric loxP system via the BKE33780 strain. The gene deletions were confirmed by sequencing.

TABLE 1
Deletion strains engineered for use in Bacillus Autolysis system

| Strain | Property of the gene |
|---|---|
| B. subtilis BMV9 ΔsdpI | Immunity protein, protection |
| | against SdpC |
| B. subtilis BMV9 ΔsdpR | Regulatory protein |
| B. subtilis BMV9 ΔsdpIR | Immunity protein and regulator |
| B. subtilis BMV9 ΔsdpABC-IR | Deletion of the operon |
| B. subtilis BMV9 ΔskfABCEFGH | Deletion of the operon |
| B. subtilis BMV9 ΔskfE | ABC transporter, export of SkfA |
| B. subtilis BMV9 ΔskfF | ABC transporter, export of SkfA |
| B. subtilis BMV9 ΔskfG | unknown |
| B. subtilis BMV9 ΔsigW | sigW - stress response in |
| | presence of SdpC |
| B. subtilis BMV9 | Deletion of the operon; stress |
| ΔsdpABC-IR ΔsigW | response in |
| | presence of SdpC |

c) Inducible Expression System for Selective Expression of SKF and SDP Killing Factors

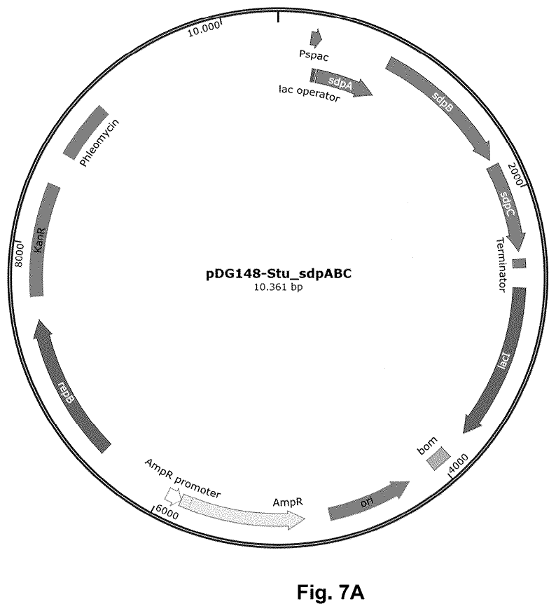

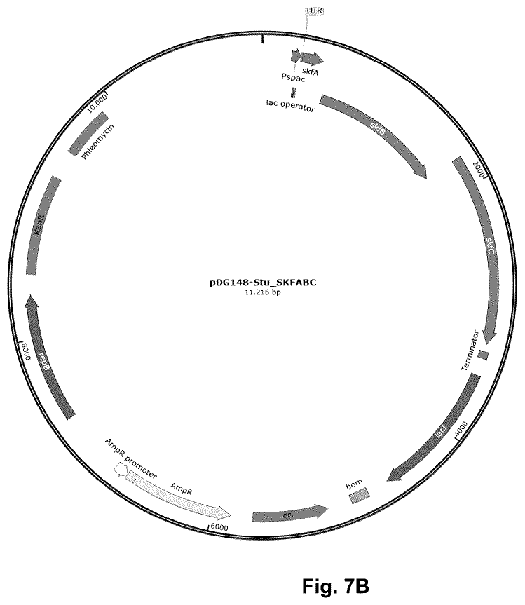

Multiple plasmid systems were tested for inducible expression of selected genes. For example, the gene encoding the Sdp killing factor and its maturation were cloned with or without the 3′ untranslated region (UTR) into the pDG148-Stu plasmid under the Pspac that allows for IPTG inducible expression (SEQ ID NOS. 27, FIG. 7A). Similarly, the skfABC genes (SEQ ID NO: 28) were cloned into the pDG148-Stu vector (FIG. 7B). Additionally, skfABCFGH genes were cloned into pMutin plasmid (SEQ ID NO: 29, FIG. 7C). The plasmids were transformed into the parent BMV9 strain and BMV9 ΔsdpI::erm and tested for inducible lysis as provided in Example 3.

d) ClpE Promoter Exchange

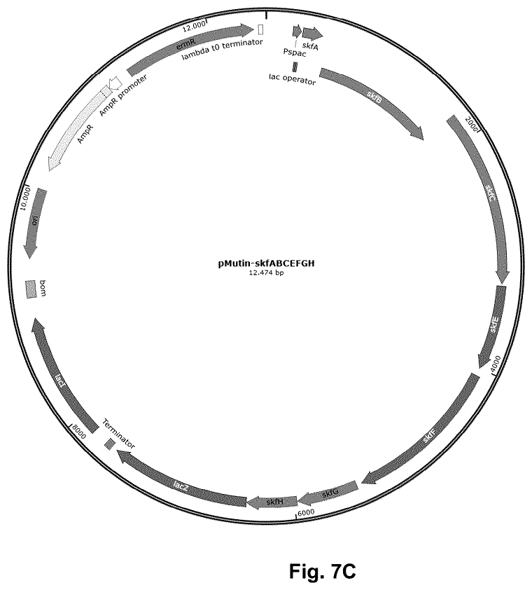

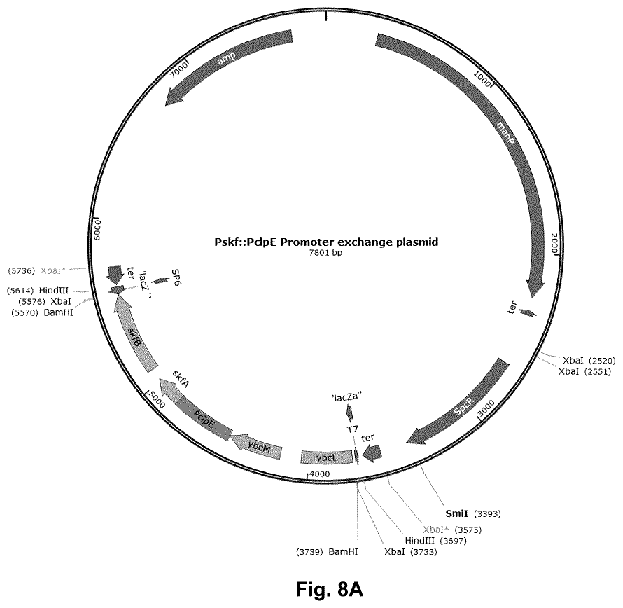

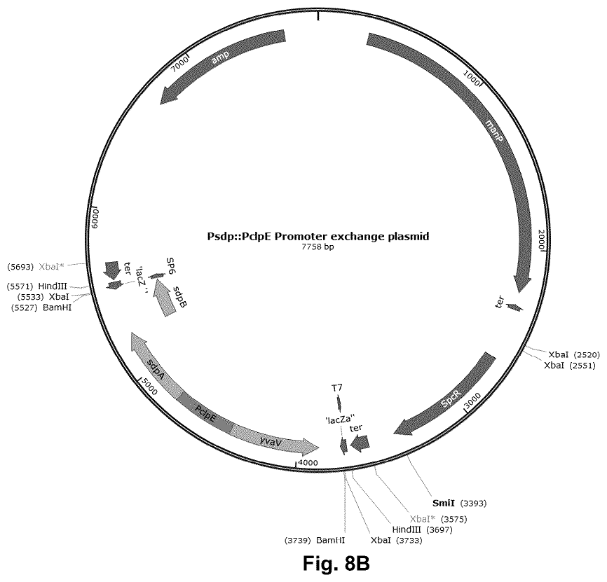

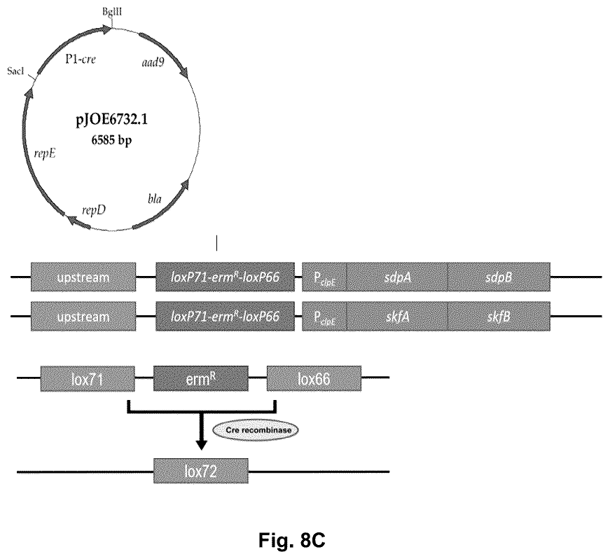

An example is the promoter of clpE gene, which is strictly repressed by thermosensitive CtsR repressor. An increase in temperature inactivates CtsR resulting in a strong induction of clpE gene expression. In order to achieve a promoter exchange between the native sdp/skf promoters and P_{clpE}, the P_{clpE} promoter was exchanged against the P_{skf} or P_{sdp} promoter. The fragments were inserted by Gibson cloning into pJOE6743.i plasmid, that allows for recombination using a Cre recombinase system and mannose counter-selection (see FIG. 8A for a schematic of the Gibson cloning for P_{clpE}::P_{skf} (SEQ ID NO: 30) and 8B for P_{clpE}::P_{sdp} (SEQ ID NO: 31). FIG. 8C provides a schematic of the Cre-recombinase system used for chromosomal integration of the chimeric fragment. The plasm id was transformed into B. subtilis BMV9 strain and colonies were picked using mannose counterselection. Promoter exchange was confirmed by sequencing. Three separate sets of strains were created by this method: 1) BMV9 P_{clpE}::P_{sdp} 2) BMV9 P_{clpE}::P_{skf}, and 3) BMV9 P_{clpE}::P_{sdp}; P_{clpE}::P_{skf}. Inducible cell lysis was tested as provided in Example 3.

### Example 3: Autolysis Plate Assay

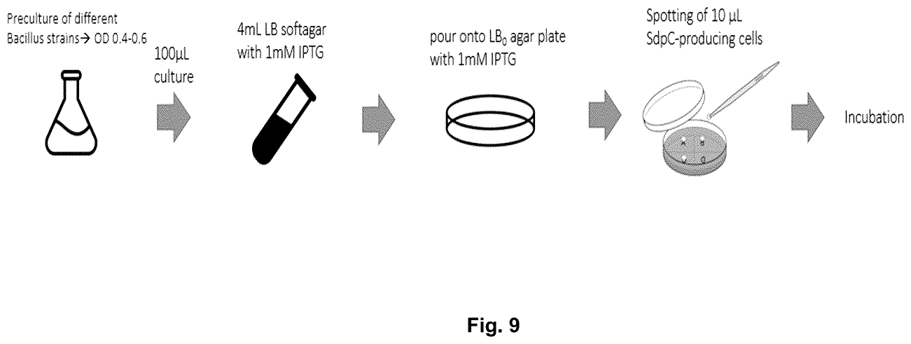

Since detection of lysis in dense cultures can be challenging, a plate assay was developed to check for induction of killing factors. For this WT B. subtilis was grown to an optical density (OD₆₀₀) of between 0.4-0.6. A volume of 100 μL of the culture was used in 4 mL of warm LB soft agar and mixed. The soft agar was poured onto an LB agar plate containing 1 mM IPTG. The engineered and control strains were inoculated from cultures and allowed to grow to an OD₆₀₀ of 1. About 20 μL of the culture was spotted on a sterile disc and placed on the soft agar and the plates were incubated at 37° C. O/N (see FIG. 9 for a schematic of the method used).

### Example 4: Effects of Deletion of SigW on the Cell Lysis System to Improve Cell Lysis

SigW induces a secondary resistance mechanism against the SDP toxin. Therefore, in order to further test the impact of SigW on lysis, strains were made as provided in Table 1 of Example 2, with the sigW gene deleted in the context of BMV9 (control) and BMV9 ΔsdpABC-IR strains. Strains were constructed using the same protocol as provided in Example 2.

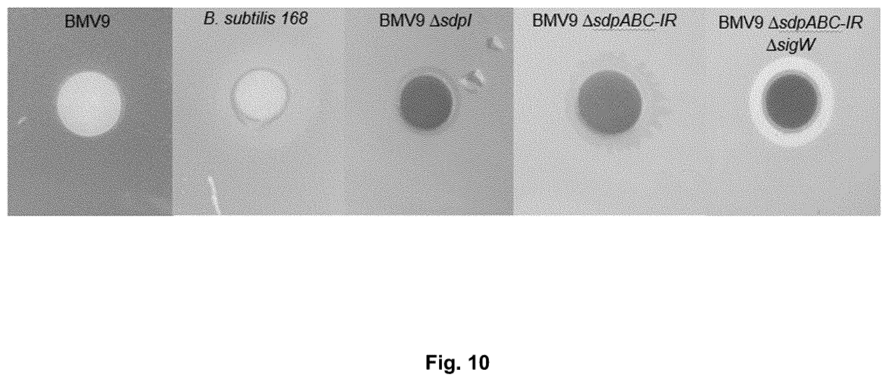

Plate lysis assays were conducted as in Example 3 and results are shown in FIG. 10. Results show a zone of clearance that is enhanced with respect to BMV9 ΔsdpABC-IR alone.

### Example 5: Deletion of Additional Factors to Improve Cell Lysis

One or more additional genes including but not limited to yfhL, yknW, yknX, yknY, yknZ, clpE will be deleted as provided in Example 2(b) and tested for impact on cell lysis post induction. Deletions will be made in context of strains generated in Example 2b and WT Bacillus subtilis strains. Strains will then be tested using the plate lysis assay as described in Example 3.

### Example 6: Overexpression of Additional Factors to Improve Cell Lysis

One or more additional genes affecting lysis including but not limited to SipT/SipS, CsaA will be overexpressed either constitutively or under inducible conditions and tested for impact on cell lysis post. Overexpressing strains will be made in context of strains generated in Example 2b and WT Bacillus subtilis strains.

Strains will be tested using the plate lysis assay described in Example 3.

### Example 7: Plate Lysis Assay to Characterize Cell Lysis Influencing Factor abrB

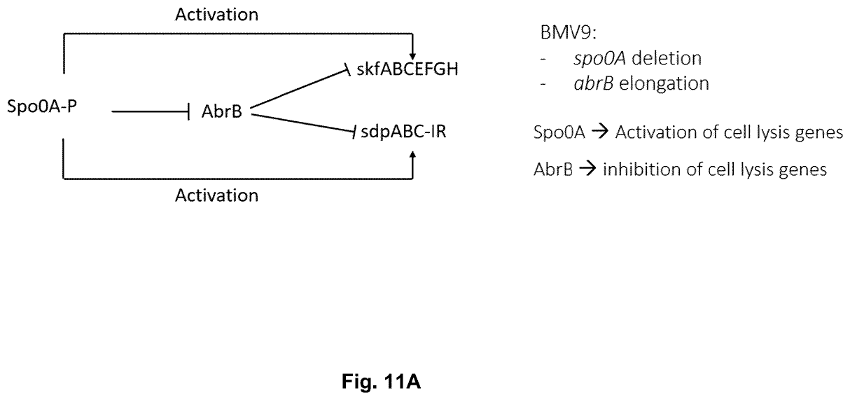

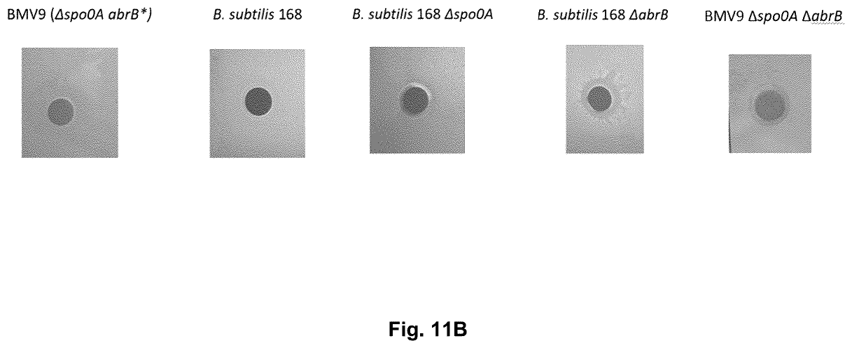

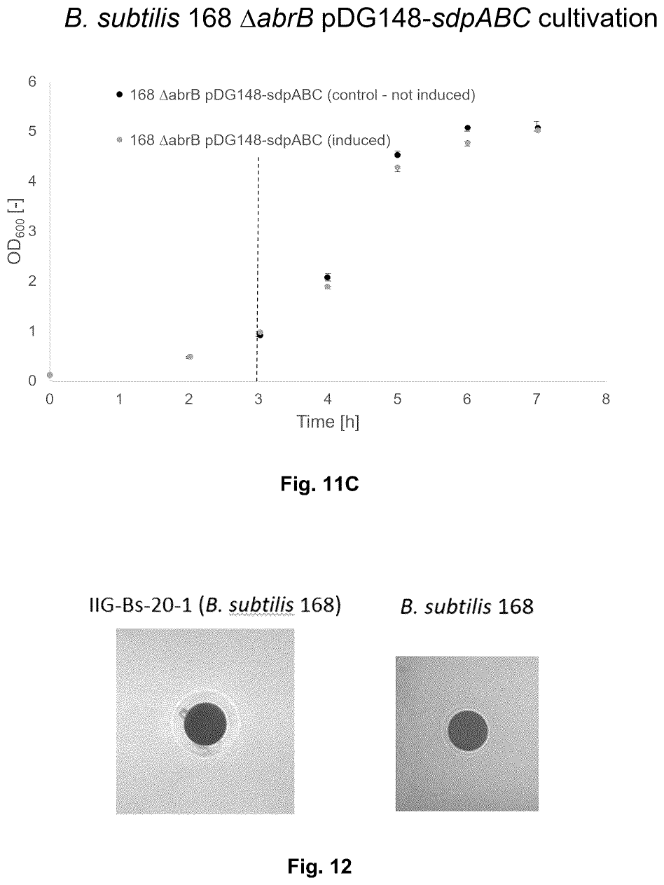

Plate lysis assay were conducted essentially as provided in Example 3, to further characterize factors that influence cell lysis. The zone of inhibition for the following strains were studied: control strains: B. subtilis 168 and BMV9, deletion strains in the context of B. subtilis 168 including Δspo0A, ΔabrB and Δspo0A ΔabrB, and deletion strain in the context of BMV9: Δspo0A abrB*. “abrB*” refers to a mutation of the stop codon in abrB gene, which results in an eleven amino acids C-terminal elongation of the AbrB regulator. Spo0A protein negatively regulates the regulatory locus of abrB, which controls the expression of many genes associated with the onset of sporulation (see FIG. 11A). Results shows that ΔabrB cells are effective in inducing enhanced cell lysis (see FIG. 11B).

This was further confirmed using the liquid cell lysis system. B. subtilis 168 ΔabrB pDG148-sdpABC cells were grown for 3 hrs. One set of cells were induced with IPTG. Absorbance of the cultures at A600 was measured at regular intervals. Results are provided in FIG. 11C.

### Example 8: Impact of a Reduced Genome on Cell Lysis

B. subtilis 168 genome is home to a number of prophages and gene clusters that do not impact the growth rate or cell morphology. For instance the genome comprises about nine prophages, and seven antibiotic biosynthesis gene clusters. Prophages of Bacillus subtilis include φ29; SP-15; SP50; φ1, φ2, φ14; β22; SPP1; SP3; SPO1; TSP-1; SPα (=PBSX); SPβ (=PBSY). Silent prophages and other genes responsible for lysis can be used to induce targeted cell lysis.

In order to study the impact of deleting these genes from the genome on cell lysis, a genome reduced version of B. subtilis 168 was generated which lacked the nine prophages, seven antibiotic biosynthesis gene clusters and two sigma factors for sporulation. The resulting strain genome was reduced from 4215 to 3640 kb and has a genotype Bacillus subtilis 168 P_{mtlA}-comKS trp⁺ Δ[SPβ] Δ[skin] Δ[PBSX] Δ[proϕ1)] Δ[proϕ2] Δ[proϕ3] Δ[proϕ4] Δ[proϕ5] Δ[proϕ6] Δ[proϕ7] Δ[pks] Δ[manPA-yjdF-yjdGHI-yjzHJ] Δ[sboAX-albABCDEFG] ΔppsABCDE ΔbacABCDEF Δ[ytpAB-ytoA] Δ[sdpABCIR] Δ[bpr-spoIIGA-sigEG] Δ[ntdABC-glcP]. The strain exhibited growth rate and cell morphology similar to the B. subtilis 168 parental strain.

Plate lysis experiments were conducted similar to Example 3. The reduced strain heretofore referred to as IIG-Bs-20-1 (B. subtilis 168) was able to affect cell lysis similar to B. subtilis 168 ΔabrB (see FIG. 12).

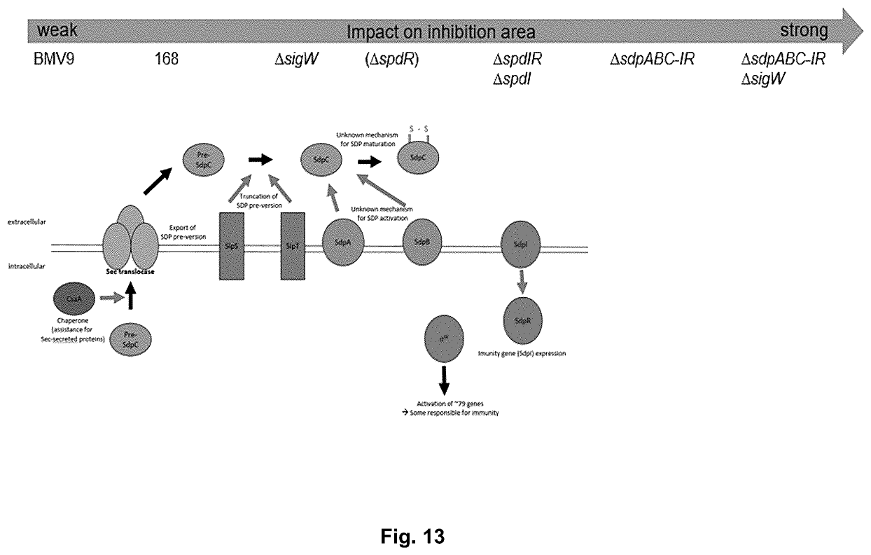

Taken together the data provided in Examples 4-8, suggested that multiple deletion strains are effective in influencing cell lysis, ranging from weak effect to strong effect (see FIG. 13). These deletions and mutants of these genes can individually or in combination can be used to effect cell lysis.

### Example 9: Proteomic Analysis

In order to further determine proteins that may impact cell lysis a proteomics approach was taken. The underlying motive for proteome analysis was to identify proteins expressed during cell lysis on agar plates. This analysis could give insights into the expression of proteins/enzymes that actively lyse the cells or are responsible for immunity against them. These results can be used to improve lysis and to understand the lysis system.

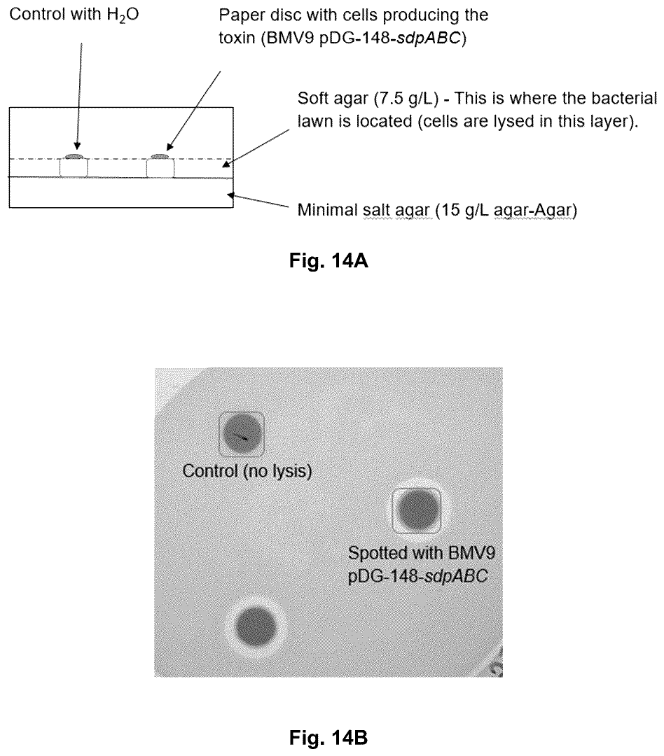

On this basis, plate lysis assays (see FIG. 14A) were conducted on BMV9 ΔsdpABC-IR ΔsigW, BMV9 ΔsdpABC-IR and control strains as provided in FIG. 14B. Areas as marked in FIG. 14B were sent for proteomic analysis. Analysis is undergoing but data showed small amounts of SdpC in the test samples compared to the control.

Further proteomic study is conducted using BMV9 ΔsdpABC-IR ΔsigW vs. BMV9 ΔsdpABC-IR strains.

### Example 10: SdpC Purification and Analysis

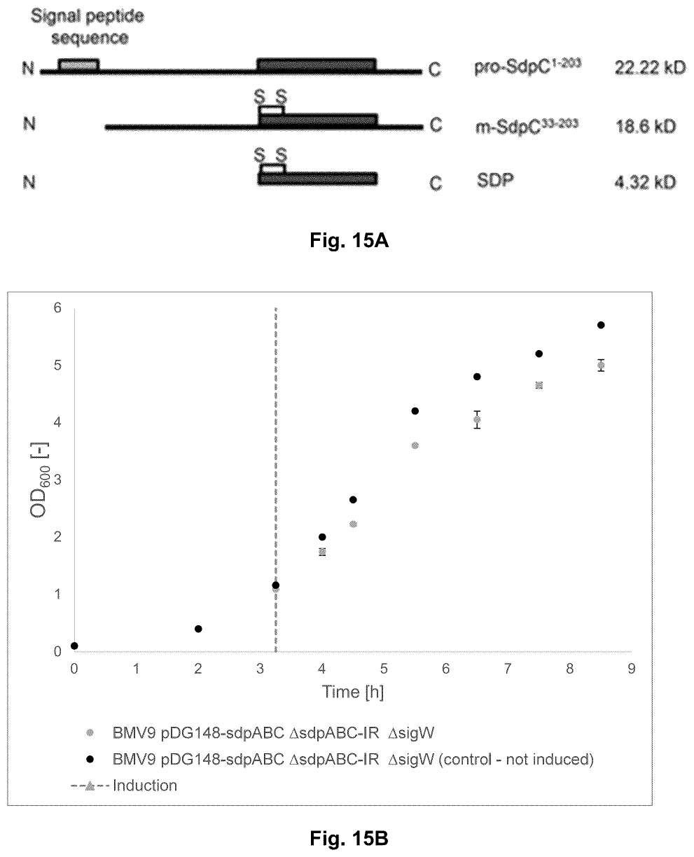

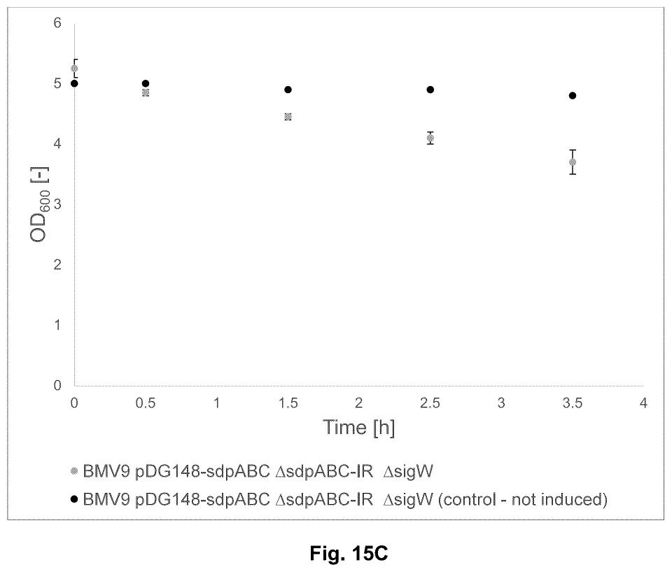

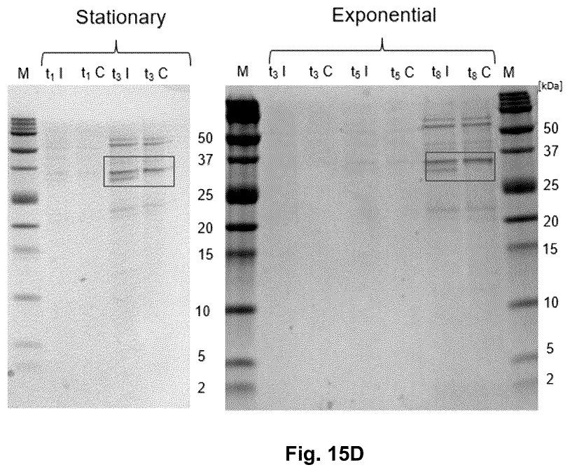

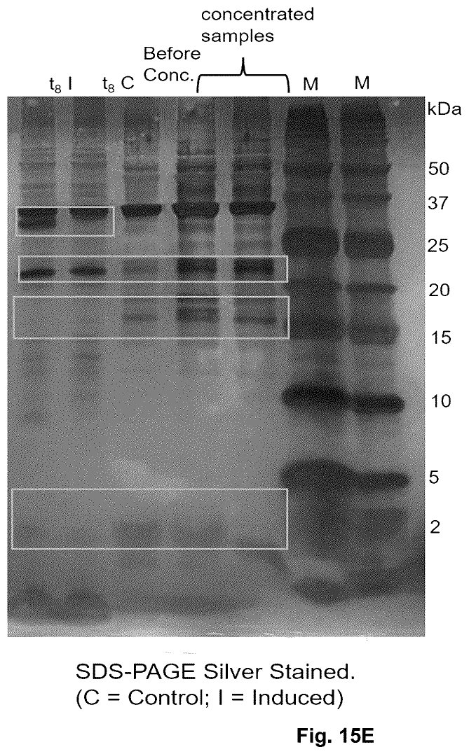

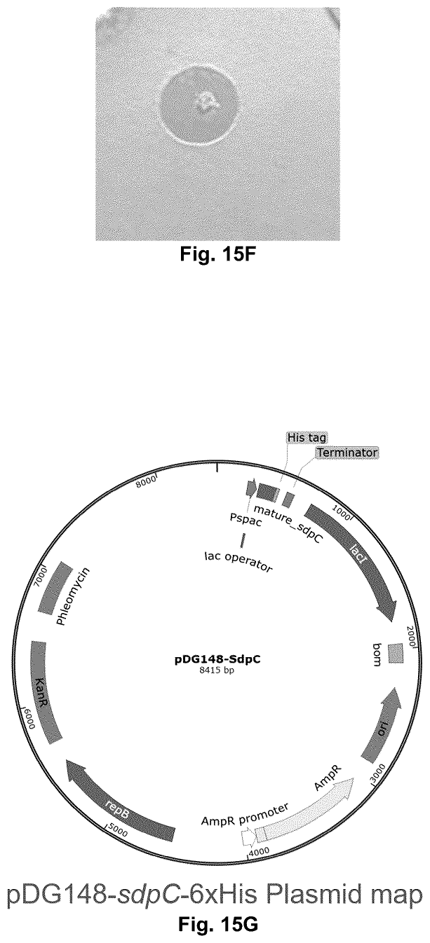

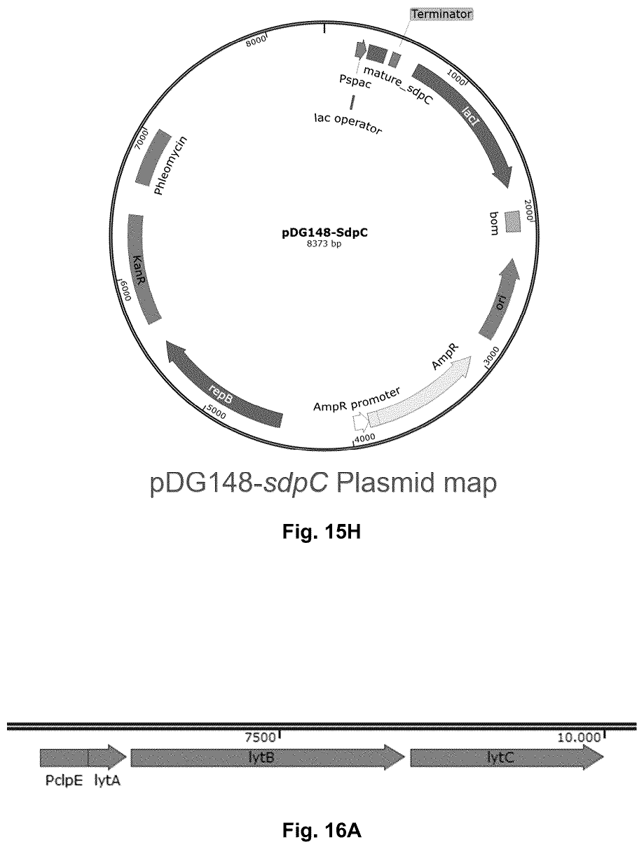

In order to further characterize the role of SdpC on cell lysis, attempts were made to purify the cell lysis protein SdpC. Using the purified SpdC toxin, the amount of protein required for activating lysis can be identified. FIG. 15A provides a schematic of the different stages in protein expression of SdpC.

Cells for use for purification were grown using shake flask cultivation. Growth curves of relevant strains are provided in FIGS. 15B and 15C, and the corresponding SDS-PAGE analysis is provided in FIG. 15D. The supernatant from these cultures was concentrated (600m L reduced to 30 mL) and run on an SDS-PAGE and silver stained (FIG. 15E). The concentrated supernatant was used for plate lysis spot test. Potential bands corresponding to SdpC were cut-out for confirmation with mass spectrometry.

The concentrated supernatant was also used for plate lysis analysis. For this Spotted 2 μL of concentrated supernatant was spotted on BMV9 ΔsdpABC-IR ΔsigW (softagar). A clear zone of inhibition was visible (see FIG. 15F), confirming that SdpC is involved in enhanced lysis.

Attempts were also made do purify SdpC using a His-tag followed by purification using a His-Trap column. Plasm id constructs used for this attempt are provided in FIG. 15G. Expression was also tested using a non-His tagged version (plasmid in FIG. 15H) transformed into various mutant strain backgrounds. However, these attempts have heretofore been unsuccessful.

### Example 11: Additional Genes being Tested for Cell Lysis

Additional genes that may impact or promote cell lysis are being explored. FIGS. 16A and 16B provide additional genes of the lytABC operon that may be involved in cell lysis, and promoter exchange constructs that may be used to express these gene. Of these genes only lytC has been demonstrated to mediate cell lysis, as deletion of the gene drastically reduces cell lysis. lytD and lytG encode for minor peptidoglycan hydrolases, while lytC may be a critical peptidoglycan hydrolase that mediates autolysis of vegetative cells—see Table 2. These genes are tested using the P_{clpE} heat inducible lysis system or the P_{mtlA}—Mannitol inducible promoter 0.5% (w/v) system as provided earlier.

Additionally, holin-like proteins, for example BhlA and BhlB and autolysins like CwlA are also being explored for their impact on cell lysis.

TABLE 2
List of lysis genes to be tested for their role in cellular lysis.

| Gene | Enzyme/Protein | Function |
|---|---|---|
| lytA | | secretion of major autolysin LytC |
| lytB | | modifier protein of major autolysin LytC |
| lytC | N-acetylmuramoyl- | major autolysin, cell separation, |
| | L-alanine amidase | wall turnover |
| bhlA | holin-like protein | regulation of cell lysis |
| bhlB | holin-like protein | regulation of cell lysis |
| cwlA | N-acetylmuramoyl- | Minor autolysin |
| | L-alanine amidase | |

### Example 12: Exploring Stronger Promoter Systems to Promote Lysis

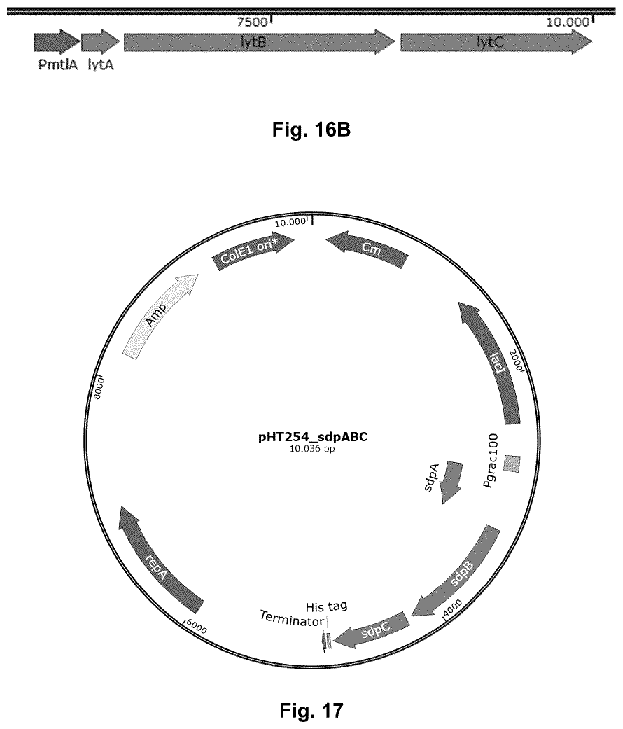

Additional plasmid-based expression systems with stronger expression rates (different promoters) are being explored. One such system is provided in FIG. 17. Stronger promoter systems provide a means to generate higher concentrations of signal molecules, increasing their chances of reaching their intended targets and achieving the desired biological responses. In the previous experiments in liquid culture none or very low cell lysis was observed. The concentration of toxins is probably a limiting factor for cell lysis. With a stronger expression, this hurdle can be overcome and may lead to stronger effects on the cells to be lysed.

SEQUENCES

| | | Protein/ | | |
|---|---|---|---|---|
| # | Source | DNA | Sequence | |
| 1 | Bacillus | DNA | ATGACTATATGTTTCCTATTATTTTCTTCTTATTACTTTAGCAATATTTCACCTCAGAATCCACTGTTCA | sdpA |
| | subtilis | | AAAAAAATTTTTTGCAACAATTGTCTCCCCAAGGCTTTGGCTTTTATAGTAAAAGCCCTACAGAAGAAAA | |
| | | | CATTTCATTTCACACAAAAGAAAATTTAAAGTTACCTAATGCACTTCCCAATAATTTTTTTGGGATAAAA | |
| | | | AGAGAAGGAAGAGTTCAGGCAATAGAATTAGGCAAAATTGTAGAGAATATCGATCCAAAGAATTGGAAAA | |
| | | | CTTGTGAAAACAACAACTCCTGCACAAATTTAGAGAAACAAATAAAGCCTATTAAGGTTATAAAAAATGA | |
| | | | AGATTATATACATCTTAGCAAAGGAGAATACCTAATATATCGCCAAAAACCACTCTCATGGTATTGGATA | |
| | | | GACTTTAAGCAAACTACCTCTTTTGAAAGAAAGGTGCTAAAAATAAAAATAGTATGA | |
| 2 | Bacillus | DNA | ATGAAGATATTAAATAGTTTAGAAGGTTATATTGACACCTATAATCCATGGAAAAATACATATGCACTTT | sdpB |
| | subtilis | | TTAGAAGTTTACTTGGTTTCTCAACATTACTAGTACTATTATTCAATAGTACTGATATTTTATTTAGTTA | |
| | | | TAGTGCAAATAATGTCACATGTGAAAATGTCTATATCCCTACCGCTTTTTGTTTTGCTAAAGAATATAGT | |
| | | | ATCAATTTTGAGATTATAAGATACTTAATGATTTTTATATTAACCTTAGTGGTTATAGGGTGGAGACCTA | |
| | | | GATTTACCGGTTTATTTCACTGGTATATTTGCTATAGTATTCAAACTTCAGCTTTAACTATCGATGGTGG | |
| | | | AGAGCAAATTGCAACTGTTCTTTCTTTTCTTATATTACCTGTTACATTATTAGATTCAAGGCGAAATCAT | |
| | | | TGGAATATAAAGAAAAACAATAATGAATCTTTCACAAAGAAGACAGTATTGTTTTATATAATGACAATAA | |
| | | | TTAAAATTCAAGTTTTTATCATTTATTTAAACGCAGCTTTAGAGCGATTGAAAAATAAAGAGTGGGCAGA | |
| | | | AGGAACAGCAATTTACTATTTCTTTTCTGATCCGGTGTTTGGATTACCTGAATATCAACTTAACTTAATG | |
| | | | AATCCACTACTTGAAAGCAATTTTATTGTTGTCATCACTTGGTTAGTAACTATTTTTGAGTTGTTCTTAG | |
| | | | CAGCAAGCATAATTTCAAATATCAGAATAAAGAGAATTGCCCTTGTTTTGGGAATATTATTTCATATTGG | |
| | | | GATAATATTCAGCATTGGTATTGTAAGTTTTGGCTTGATCATGATATCAGCATTAATTATATATCTGCAT | |
| | | | CCTGTACAACAAAATATCACTATGAATTGGTGTTCTCCTTTATTTAAATATATATATGTAAAAGGAAAGA | |
| | | | GAAATTTCAAAAGAATAGGAGGTGAATCAGTCAAGTTTCTTACAAAATTGTTTCATAGCTAA | |
| 3 | Bacillus | DNA | TTGAAAAGTAAATTACTTAGGCTATTGATTGTTTCCATGGTAACGATATTGGTTTTTTCATTAGTAGGAC | sdpC |
| | subtilis | | TCTCTAAGGAGTCAAGTACATCTGCTAAAGAAAACCATACATTTTCTGGAGAAGATTACTTTAGAGGACT | |
| | | | TTTATTTGGACAAGGGGAAGTTGGTAAATTAATTTCAAACGATTTGGACCCTAAACTCGTAAAAGAGGCA | |
| | | | AATAGTACAGAAGGTAAAAAGTTAGTAAATGATGTAGTCAAATTTATAAAAAAAGATCAACCACAATATA | |
| | | | TGGATGAATTGAAACAATCGATTGACAGCAAAGACCCTAAAAAACTCATTGAAAATATGACCAAAGCAGA | |
| | | | CCAACTTATCCAAAAATATGCTAAGAAAAATGAAAACGTAAAATACTCTTCTAATAAAGTTACTCCATCT | |
| | | | TGTGGGCTTTATGCCGTCTGTGTAGCAGCTGGATATTTATATGTTGTGGGCGTTAACGCAGTTGCATTAC | |
| | | | AAACGGCTGCCGCAGTAACAACTGCAGTGTGGAAATACGTTGCCAAATATTCCTCTTCAGCTTCTAATAA | |
| | | | TTCTGATTTAGAAGCGGCTGCTGCAAAAACCCTAAAATTGATTCATCAATAA | |
| 4 | Bacillus | DNA | ATGAATAATGTTTTCAAAGCTATATCAGATCCAACAAGGAGAAAAATTTTGGATTTGTTAAAAGGAGGGG | sdpR |
| | subtilis | | ATATGACTGCGGGAGATATTGCTGAGCATTTTAATATCAGCAAACCGAGTATTTCGCACCACTTGAATAT | |
| | | | TTTAAAGCAAGCGGAAGTCATAAGTGACCATCGCAAAGGACAATTTATTTATTACTCTCTAAATACAACA | |
| | | | GTACTTCAAGACTCAATAAATTGGATGCTTAACTTTATAAATAAAGGGGATAACGATTTATGA | |
| 5 | Bacillus | DNA | ATGAAGAAAAATATAATTTCCATAATTATTGTATGTTTGAGTTTCTTGACTTCAATTATATTATATCAAT | sdpl |
| | subtilis | | ACCTTCCAGAAGAAATACCGATACAATGGTCAGGAAATAAACCGGCTGCAATTGTATCTAAACCTTTAAC | |
| | | | AATATTTATTATACCTGTTGTTATGCTTATTTATTATCTTACTTTTTATATGTTAACTATTAAGTCTACA | |
| | | | CAAAAAAATAAAGCATTGCTTTTCCTAGCTTCTAATAATATGTTAATCCTTTTATATATTTTACAACTCT | |
| | | | CAACTCTATTGATAAGTTTAGGATACGAAGTGAATATTGATTTAATAATAGGGCTTGGTGTTGGTATCTT | |
| | | | TTTAATTATTGGTGGAAATTCTATGCAGCTAGCAGAACAAAACCATCTTATTGGATTGAGGACACCTTGG | |
| | | | ACATTAAAAGATGAGACTGTTTGGAAACTGGGAAATCGCTTTGCCTCTAAAGTACTTGTAGTGTGTGGTT | |
| | | | TTATAATAGCTGTTCTTTCATTTTTTACAGGAGAATATATAATTCTTATTATGATTGTGCTAGTTCTTCT | |
| | | | AGCATTAGTTATTTCAACACTAGCTTCATATCATTATTACAAAAAATTAAACGGTTCACGTTGA | |
| 6 | Bacillus | DNA | ATGAAAAGAAACCAAAAAGAATGGGAATCTGTGAGTAAAAAAGGACTTATGAAGCCGGGAGGTACTTCGA | skfA |
| | subtilis | | TTGTGAAAGCTGCTGGCTGCATGGGCTGTTGGGCCTCGAAGAGTATTGCTATGACACGTGTTTGTGCACT | |
| | | | TCCGCATCCTGCTATGAGAGCTATTTAA | |
| 7 | Bacillus | DNA | ATGTCATATGATCGTGTTAAAGATTTTGATTTGCCAGAGTTAGCGGTTCATTTACAGCCTCATGGTGCTG | skfB |
| | subtilis | | TAATGATTGATAGAAAAAGTATGTTTTATTTCAGACTCAGTGGACGTGGAGCACAGTTGGCGTTTCTGTT | |
| | | | ATCAAAAAACAAAAATCTTCATAAAACGGCACGTATTTGGGAGATTATGAAAAAAGAAGAGATGAGTGCT | |
| | | | GACCAATTAAAAGAAGAGCTTAGTGCCCATCCATTTACGGAGGCTTGGACGGAAGGGCTGTTAGATCAGC | |
| | | | CTTTACACGTTTCGGGTTCGCTAGATTCATATTTACCTATTAGTTGTACCTTACAGTTGACAAATGCTTG | |
| | | | TAATTTAAGCTGTTCGTTTTGCTATGCCAGCTCAGGTAAACCATATCCTGAGGAATTATCTAGTGAACAA | |
| | | | TGGATATTGGTTATGCAAAAGCTAGCAGCCCATGGAGTTGCTGATATTACGCTGACCGGGGGTGAAGCAA | |
| | | | AGCTGATCAAAGGGTTTAAAGAATTAGTCGTTGTTGCAAGTTCGTTGTTTACCAATGTGAATGTATTTAG | |
| | | | TAATGGATTGAACTGGCGAGATGAAGAAGTTGAATTACTTAGTCACTTAGGCAATGTTTCTGTACAGATT | |
| | | | AGTATTGATGGTATGGATAATACACATGACCAATTAAGAGGCAGAAAAGGCGGCTTTAAAGAAAGTATGA | |
| | | | ATACCATTAAAAAATTATCAGAAGCAAACATTCCAGTGATTGTCGCTATGACCATTAATGAGTCCAATGC | |
| | | | TGATGAAGTATCAGATGTGGTGGAGCAATGTGCCAATGCGGGTGCTTTTATCTTTCGTGCAGGAAAAACG | |
| | | | TTATCTGTTGGACGTGCGACAGAAGGCTTTAAGGCTTTGGATATTGATTTTGAAGAAATGGTTCAAATAC | |
| | | | AGCTTAGGGAAGCACGTCATAAATGGGGAGATCGCCTGAACATAATTGATTGGGAGCACGAAGAGAGTTC | |
| | | | GTTCACAACAGATTTTTGTACACCAGGATATCTTGCTTGGTATATAAGAGCAGATGGATACGTAACTCCT | |
| | | | TGCCAATTAGAAGATTTGCCGCTAGGGCATATTTTAGAAGATAGCATGGCTGACATTGGTTCACCTGCTC | |
| | | | GTTTGCTTCAGTTAAAATGTGAAGCAAAAAATTGCAAATGTATAGGGAAAATTGAGCTATCTGAACCGGA | |
| | | | CTTACCTTTTCAAAAAGAAGTCAAGGCAGGGATTCAGGAATGA | |
| 8 | Bacillus | DNA | ATGAATAGTCTATCATTGGTGTTCTGGAGTATTTTAGCAGTTGTTGGATTACTGTTATTTATTAAATTCA | skfC |
| | subtilis | | AACCCCCAACAATTGCTTCACTACTCTTAAGCAAAGATGAGGCAAAAGAAATAAGCATTCAATTTATAAA | |
| | | | AGAGTTTGTTGGGATAGATGTAGAGAACTGGGATTTTTATTCAGTATATTGGTATGACCACGATACAGTA | |
| | | | AATAAACTTCATCACTTAGGCATACTTAAGAAAAATAGAAAGGTTTTATATGATGTTGGGTTGGTCGAAT | |
| | | | CATGGAGAGTCCGTTTCGTTCACCAGAATCAATCATTTGTAGTTGGTGTCAATGCCAATCGAGAAATCAC | |
| | | | TTTTTTTTATGCGGATGTTCCGAAAAAAACCCTTTCGGGGAAGTTTGAACAAGTTTCTCCAGAGACACTC | |
| | | | AAGCAGAGGTTAATGGCTTCACCTGATGGACTTTGGTCTAGAGCAAATATGACTGGTACTGGTAAAAAAG | |
| | | | AGGAGGATTTTCGCGAGGTCAGTACTTATTGGTACATAGCGGAAGCGGGAGATATTCGGCTCAAAGTGAC | |
| | | | TGTTGAATTACAGGGCGGCCGAATTTCTTATATTGGTACTGAACAAGAAATACTAACAGATCAAATGAGT | |
| | | | AAAGTCATTCGAGATGAACAAGTGGAATCGACATTCGGAGTATCTGGTATGCTGGGTTCAGCTTTAGCGA | |
| | | | TGATCCTTGCGATTCTCATCCTTGTATTTATGGATGTGCAAACAAGCATAATCTTCAGTCTTGTTCTGGG | |
| | | | TTTGTTGATTATAATATGCCAGTCATTGACGCTGAAAGAAGATATTCAATTAACAATTGTAAATGCTTAT | |
| | | | GATGCAAGAATGAGTGTCAAAACGGTCAGTTTATTAGGTATTTTGTCTACACTTCTTACAGGATTATTAA | |
| | | | CAGGATTTGTAGTATTTATATGTTCATTGGCAGGAAATGCGCTTGCTGGTGATTTTGGATGGAAAACGTT | |
| | | | TGAACAACCAATAGTTCAGATTTTCTATGGAATAGGAGCAGGGCTCATTAGTTTAGGAGTGACTTCTCTG | |
| | | | CTGTTTAACTTATTGGAGAAAAAGCAATATTTACGAATTTCACCTGAGCTTTCTAACCGAACTGTCTTTC | |
| | | | TATCAGGTTTTACCTTTAGGCAAGGATTGAATATGAGCATACAAAGTTCAATTGGAGAAGAGGTCATCTA | |
| | | | TCGGCTATTAATGATTCCAGTCATTTGGTGGATGAGTGGAAATATCCTCATCTCCATTATTGTATCTTCC | |
| | | | TTTTTATGGGCGGTGATGCACCAAGTAACTGGATATGACCCAAGGTGGATACGTTGGCTGCATCTATTTA | |
| | | | TATTCGGTTGCTTTCTGGGAGTTCTCTTCATCAAATTTGGTTTTATTTGTGTATTAGTAGCTCATTTCAT | |
| | | | TCATAATTTAGTACTCGTCTGTATGCCGCTGTGGCAGTTCAAGCTTCAGAAACATATGCATCATGATCAG | |
| | | | CCAAAGCATACTTCACTCTAA | |
| 9 | Bacillus | DNA | ATGCAATTGATGCAAGTACAAAATTTAAGCAAATGCTATCGAAATGGTGATGGGGTTGAACATTTGTCTT | skfE |
| | subtilis | | TCTCAATTCAACGAGGAGAAATCGTGGCGCTATTGGGGCCAAATGGAGCTGGAAAAACAACGACAATTCG | |
| | | | ATGTTTGACAGGTCTCTATAAGCCGGATAAAGGAGATATCCTCATTGAGGGTTCTCCTCCAGGAGATATA | |
| | | | AATGTTCAAAAAAAAGTCGCGCTTATTCCTGATCAGCCATATTTGTATCCCGCTTTAACTGCTGCTGAAC | |
| | | | ATATTCAGTTTAGGGCAAGAGGATATCATCCAGGTAAAAAAGATGTAAAAGAAAGGGTTTATCATGCATT | |
| | | | GAAGGAAGTGCACTTGGAGGAGAAAGCAAACCAATTGTGTGGTCAGCTGTCAAGAGGCCAAAAGCAGCGG | |
| | | | GTTGTCCTGGCGGGGGCGATTGTTCAAGACGCTTTGCTGTATATATTAGACGAACCGACTGTGGGCCTTG | |
| | | | ATATCCCATCAAAACAGTGGCTCTCTAACTGGTTAAAAACTAAAACCGATCAAGGGTGTTCAGCATTTGT | |
| | | | ATCAACTCATAGTCTAGAGTTTGTTATAGAGACAGCAGACAGGGTGATATTAATTAGAGACGGCAAACTC | |
| | | | ATGCAGGATTTGTATGTTCCTCAATTCGAAGAACAAGCGGAATGGAGAAAAGAAGTGATTCGTCTTCTAG | |
| | | | GGGAATGGTCAGATGAATAA | |
| 10 | Bacillus | DNA | ATGCCTTTTTTGATCATGCTCCTTTTTGTTGGGGCCATCGGATTTCAAGTAAGTTTTGTTTCTAGATCTA | skfF |
| | subtilis | | CGACATGGGATATGAGTATTGCTGGTTGGGTACTTACAGGTGTTTTTATCCTTTATACGGCATTTGGACT | |
| | | | TTTTTCAAATCGATTACCAAGTCAAATGGCAGATATTATATGGCTTTATGGCACTGCCACATCTTTTTCA | |
| | | | AAAGTTGTGTATAGTGTTTTATTTTTCAGCGTCACTTGGAAGGCGTTGCTTTGGATCATCTCAGCCATAT | |
| | | | TCGGTGATGTATTAATTGTGCTTCTGTCTGGTGATCATATCAATTTATTAGGCCGATCCATAATTTTTGT | |
| | | | AGGGCTCTTTTTTATCGCTGAAGTGTGGTTGATGTCGGTCTCTTGTGCCAGAACAGTGAAGAAAATGAAA | |
| | | | AGGGTATACGTTTTAGTCTTTCTTCTAATGTTAGGCATTTACTCCATATGTCTTTATCGCTTCTTCTTTC | |
| | | | TCCAACATTCATCTGGGATATGGGAGAGCATTGCCCGTTTTATTAGTGGCGTTGGATTAGTGTTTGATAC | |
| | | | ATTGTCACCGTTGTATGTGGTTGTATTTATTGGGATTATCACAGTCTCTTTTATGACAATTGCTTTTACG | |
| | | | AGCCGTCAGGTAGAAATGAAGGAATCGTTGGTGAAAGAAGCTGAATTCTGGGAGGAATTTCAAGAACGTC | |
| | | | AATTTGGTTCAGGTCAAATTATACAGAAACCAAAAACGACTTGGTGGGGCTTGCAAGGTCTAAATGGCAT | |
| | | | TTGGTCTTTTCTGTGGTTGGAACTGTTGCTTTTTAAAAAATATTTATTTTTTCATAGCATTCATACGGTC | |
| | | | ATGCTCAGTGGCGTCTTTTATGTCGTCATTTTCATGTATCCAGAATGGTTTTATCTTCTATTCTTTCTTA | |
| | | | TCGTCTCGGCAGTCATGTTAAGTTCCTATTATTCAGGAATTGTCAGACATTCTCAATCAGGCACCCTTCA | |
| | | | TTTATTCCCCGGTGCCCTTTGGAAAAAAATCATTATTCTAGAGCTGACGAATACAGTCTGGTTGTATATT | |
| | | | CTTTACTGTGTTTCTATTACTTTTATGGCAGTTGGGAATTTAGTTTATTGGTATATATATGGCTTAGGGA | |
| | | | TATATATATGGTTCATGACAATAAGGCTTTTTGCTTTTACCCATACAAACCGAAACGATATTAAGCTTTC | |
| | | | ATTGCCTCAATACTACAAGTCATTTTTTATGGCATTAGGCCTGAGCGGCATTTGTTTGTATGTCATTCAT | |
| | | | TTATTGACTGCTGACTGGTATACATTAGTGGTGGTCGTCTGCATAGGGAGCCTAAGTTGGTGCCTGTTTT | |
| | | | ATCGTTTCAGATAG | |
| 11 | Bacillus | DNA | ATGAATTCAAATGGTGATAAATTGTCGTTATCTGTTCAGAATTTAGCGAATACAAATGAGATCACAATTG | skfG |
| | subtilis | | TTCAAGCGATAGGTGAGCTAAAGAAATCGGGAAAAGATGCAATACCAGTTTTGGTTGAGGCCTTAAAAGA | |
| | | | GGAAGGCTCTTTGTGTAATATTGCTGCAGCTGTTTTAGGTGAGTTTGGGGAAGACGCGAGTGAAGCAGCG | |
| | | | GAGGAGCTATCATGTTTATTAAAAAGTCATGCGGAAGATACAAGAATGGCAGCGGCGATTTCATTAATGA | |
| | | | GAATCGGGAAGCCCAGTCTGCCCTTTGTCATCAAAATCGCTCAAGAAAGTGAAGGGCAATCGTGCTTTTG | |
| | | | GGCATCTTGGTGTATTGCATGGATTGATCCGTCTTGCATTGAACCTAAGATGTATAAATGTCTAAAATAC | |
| | | | GAGCATGAACATCCTTCAGGAATAGTTGCTCCGTTTGCGGCTGAAGAGGCATTAGGAAAGCTGATTGCTT | |
| | | | TTCAGCTGAAAGATAAGGAGGACTGA | |
| 12 | Bacillus | DNA | ATGAAAGATGAACAAATGTTGACTGAATGGCCAAGTCATTTACCATGGTTGAATCAATCACAGAATGATT | skfH |
| | subtilis | | TTACATTTCCAAGTGACACATATCTTCTTCTTTATTTTTGGTCAATGAGCTGTCCAAACTGTCACCAATT | |
| | | | AACAGACAAAGTCCTTCAAGATATAAAGGATATGAATGTGAAAGTAATCGGAGTACATGTCCCCTATATA | |
| | | | GAAGAAGAGAAATCTATGGAGGTTGTCTTGACGTATGCTCTTGATAGGGGACTAGCTATTCCGATTGTAT | |
| | | | TAGACCAAAACTATGAGATCGTCACAACTTGTCACGTACAAGGCATCCCCAGCTTTTGTCTATTAAGTCA | |
| | | | ATACGGTCAGATCATTACTAAAACGATGGGAGATGTTGGTTGGGATAAGATGTTAAAAAAGATTGCAGGC | |
| | | | TTGTGA | |
| 13 | Bacillus | protein | MTICFLLFSSYYFSNISPQNPLFKKNFLQQLSPQGFGFYSKSPTEENISFHTKENLKLPNALPNNFFGIK | SdpA |
| | subtilis | | REGRVQAIELGKIVENIDPKNWKTCENNNSCTNLEKQIKPIKVIKNEDYIHLSKGEYLIYRQKPLSWYWI | |
| | | | DFKQTTSFERKVLKIKIV | |
| 14 | Bacillus | protein | MKILNSLEGYIDTYNPWKNTYALFRSLLGFSTLLVLLENSTDILFSYSANNVTCENVYIPTAFCFAKEYS | SdpB |
| | subtilis | | INFEIIRYLMIFILTLVVIGWRPRFTGLFHWYICYSIQTSALTIDGGEQIATVLSFLILPVTLLDSRRNH | |
| | | | WNIKKNNNESFTKKTVLFYIMTIIKIQVFIIYLNAALERLKNKEWAEGTAIYYFFSDPVFGLPEYQLNLM | |
| | | | NPLLESNFIVVITWLVTIFELFLAASIISNIRIKRIALVLGILFHIGIIFSIGIVSFGLIMISALIIYLH | |
| | | | PVQQNITMNWCSPLFKYIYVKGKRNFKRIGGESVKFLTKLFHS | |
| 15 | Bacillus | protein | MKSKLLRLLIVSMVTILVFSLVGLSKESSTSAKENHTFSGEDYFRGLLFGQGEVGKLISNDLDPKLVKEA | SdpC |
| | subtilis | | NSTEGKKLVNDVVKFIKKDQPQYMDELKQSIDSKDPKKLIENMTKADQLIQKYAKKNENVKYSSNKVTPS | |
| | | | CGLYAVCVAAGYLYVVGVNAVALQTAAAVTTAVWKYVAKYSSSASNNSDLEAAAAKTLKLIHQ | |
| 16 | Bacillus | protein | MNNVFKAISDPTRRKILDLLKGGDMTAGDIAEHFNISKPSISHHLNILKQAEVISDHRKGQFIYYSLNTT | sdpR |
| | subtilis | | VLQDSINWMLNFINKGDNDL | |
| 17 | Bacillus | protein | MKKNIISIIIVCLSFLTSIILYQYLPEEIPIQWSGNKPAAIVSKPLTIFIIPVVMLIYYLTFYMLTIKST | SdpI |
| | subtilis | | QKNKALLFLASNNMLILLYILQLSTLLISLGYEVNIDLIIGLGVGIFLIIGGNSMQLAEQNHLIGLRTPW | |
| | | | TLKDETVWKLGNRFASKVLVVCGFIIAVLSFFTGEYIILIMIVLVLLALVISTLASYHYYKKLNGSR | |
| 18 | Bacillus | protein | MKRNQKEWESVSKKGLMKPGGTSIVKAAGCMGCWASKSIAMTRVCALPHPAMRAI | SkfA |
| | subtilis | | | |
| 19 | Bacillus | protein | MSYDRVKDFDLPELAVHLQPHGAVMIDRKSMFYFRLSGRGAQLAFLLSKNKNLHKTARIWEIMKKEEMSA | SkfB |
| | subtilis | | DQLKEELSAHPFTEAWTEGLLDQPLHVSGSLDSYLPISCTLQLTNACNLSCSFCYASSGKPYPEELSSEQ | |
| | | | WILVMQKLAAHGVADITLTGGEAKLIKGFKELVVVASSLFTNVNVFSNGLNWRDEEVELLSHLGNVSVQI | |
| | | | SIDGMDNTHDQLRGRKGGFKESMNTIKKLSEANIPVIVAMTINESNADEVSDVVEQCANAGAFIFRAGKT | |
| | | | LSVGRATEGFKALDIDFEEMVQIQLREARHKWGDRLNIIDWEHEESSFTTDFCTPGYLAWYIRADGYVTP | |
| | | | CQLEDLPLGHILEDSMADIGSPARLLQLKCEAKNCKCIGKIELSEPDLPFQKEVKAGIQE | |
| 20 | Bacillus | protein | MNSLSLVFWSILAVVGLLLFIKFKPPTIASLLLSKDEAKEISIQFIKEFVGIDVENWDFYSVYWYDHDTV | SkfC |
| | subtilis | | NKLHHLGILKKNRKVLYDVGLVESWRVRFVHQNQSFVVGVNANREITFFYADVPKKTLSGKFEQVSPETL | |
| | | | KQRLMASPDGLWSRANMTGTGKKEEDFREVSTYWYIAEAGDIRLKVTVELQGGRISYIGTEQEILTDQMS | |
| | | | KVIRDEQVESTFGVSGMLGSALAMILAILILVFMDVQTSIIFSLVLGLLIIICQSLTLKEDIQLTIVNAY | |
| | | | DARMSVKTVSLLGILSTLLTGLLTGFVVFICSLAGNALAGDFGWKTFEQPIVQIFYGIGAGLISLGVTSL | |
| | | | LFNLLEKKQYLRISPELSNRTVFLSGFTFRQGLNMSIQSSIGEEVIYRLLMIPVIWWMSGNILISIIVSS | |
| | | | FLWAVMHQVTGYDPRWIRWLHLFIFGCFLGVLFIKFGFICVLVAHFIHNLVLVCMPLWQFKLQKHMHHDQ | |
| | | | PKHTSL | |
| 21 | Bacillus | protein | MQLMQVQNLSKCYRNGDGVEHLSFSIQRGEIVALLGPNGAGKTTTIRCLTGLYKPDKGDILIEGSPPGDI | SkfE |
| | subtilis | | NVQKKVALIPDQPYLYPALTAAEHIQFRARGYHPGKKDVKERVYHALKEVHLEEKANQLCGQLSRGQKQR | |
| | | | VVLAGAIVQDALLYILDEPTVGLDIPSKQWLSNWLKTKTDQGCSAFVSTHSLEFVIETADRVILIRDGKL | |
| | | | MQDLYVPQFEEQAEWRKEVIRLLGEWSDE | |
| 22 | Bacillus | protein | MPFLIMLLFVGAIGFQVSFVSRSTTWDMSIAGWVLTGVFILYTAFGLFSNRLPSQMADIIWLYGTATSFS | SkfF |
| | subtilis | | KVVYSVLFFSVTWKALLWIISAIFGDVLIVLLSGDHINLLGRSIIFVGLFFIAEVWLMSVSCARTVKKMK | |
| | | | RVYVLVFLLMLGIYSICLYRFFFLQHSSGIWESIARFISGVGLVFDTLSPLYVVVFIGIITVSFMTIAFT | |
| | | | SRQVEMKESLVKEAEFWEEFQERQFGSGQIIQKPKTTWWGLQGLNGIWSFLWLELLLFKKYLFFHSIHTV | |
| | | | MLSGVFYVVIFMYPEWFYLLFFLIVSAVMLSSYYSGIVRHSQSGTLHLFPGALWKKIIILELTNTVWLYI | |
| | | | LYCVSITFMAVGNLVYWYIYGLGIYIWFMTIRLFAFTHTNRNDIKLSLPQYYKSFFMALGLSGICLYVIH | |
| | | | LLTADWYTLVVVVCIGSLSWCLFYRER | |
| 23 | Bacillus | protein | MNSNGDKLSLSVQNLANTNEITIVQAIGELKKSGKDAIPVLVEALKEEGSLCNIAAAVLGEFGEDASEAA | SkfG |
| | subtilis | | EELSCLLKSHAEDTRMAAAISLMRIGKPSLPFVIKIAQESEGQSCFWASWCIAWIDPSCIEPKMYKCLKY | |
| | | | EHEHPSGIVAPFAAEEALGKLIAFQLKDKED | |
| 24 | Bacillus | protein | MKDEQMLTEWPSHLPWLNQSQNDFTFPSDTYLLLYFWSMSCPNCHQLTDKVLQDIKDMNVKVIGVHVPYI | SkfH |
| | subtilis | | EEEKSMEVVLTYALDRGLAIPIVLDQNYEIVTTCHVQGIPSFCLLSQYGQIITKTMGDVGWDKMLKKIAG | |
| | | | L | |
| 25 | Synthetic | DNA | ATGAGACCTAAGCATCCGATTAAACATCAAGGCCTGCCTCAAGAAGTTCTGAATGAAAATCTGCTGAGAT | Alpha |
| | | | TCTTTGTGGCACCGTTTCCTGAGGTGTTCGGCAAAGAAAAAGTTAATGAACTGTCAAAAGATATTGGCTC | caesin |
| | | | AGAATCAACAGAAGATCAAGCAATGGAAGATATTAAACAAATGGAAGCTGAAAGCATTAGCTCATCAGAA | |
| | | | GAGATTGTTCCGAATAGCGTGGAACAAAAACATATTCAAAAGGAGGATGTTCCGAGCGAAAGATATCTGG | |
| | | | GCTATCTTGAACAACTTCTGAGACTTAAAAAGTATAAGGTTCCGCAACTTGAAATTGTTCCGAATAGCGC | |
| | | | AGAAGAGAGACTTCATAGCATGAAAGAAGGCATTCATGCGCAACAGAAAGAACCGATGATTGGAGTTAAT | |
| | | | CAAGAACTTGCGTATTTTTATCCGGAACTGTTTAGACAATTCTATCAACTGGATGCGTATCCGTCAGGCG | |
| | | | CTTGGTATTACGTTCCGCTGGGAACACAATATACAGATGCTCCGTCTTTTAGCGATATTCCGAATCCGAT | |
| | | | TGGCTCTGAAAACTCTGAAAAGACAACGATGCCGCTGTGGTAA | |
| 26 | Bos | Protein | MRPKHPIKHQGLPQEVLNENLLRFFVAPFPEVFGKEKVNELSKDIGSESTEDQAMEDIKQMEAESISSSE | Alpha |
| | Taurus | | EIVPNSVEQKHIQKEDVPSERYLGYLEQLLRLKKYKVPQLEIVPNSAEERLHSMKEGIHAQQKEPMIGVN | casein |
| | | | QELAYFYPELFRQFYQLDAYPSGAWYYVPLGTQYTDAPSFSDIPNPIGSENSEKTTMPLW | |
| 27 | Syn- | DNA | ATGACTATATGTTTCCTATTATTTTCTTCTTATTACTTTAGCAATATTTCACCTCAGAATCCACTGTTCA | sdpAB |
| | thetic | | AAAAAAATTTTTTGCAACAATTGTCTCCCCAAGGCTTTGGCTTTTATAGTAAAAGCCCTACAGAAGAAAA | C |
| | | | CATTTCATTTCACACAAAAGAAAATTTAAAGTTACCTAATGCACTTCCCAATAATTTTTTTGGGATAAAA | |
| | | | AGAGAAGGAAGAGTTCAGGCAATAGAATTAGGCAAAATTGTAGAGAATATCGATCCAAAGAATTGGAAAA | |
| | | | CTTGTGAAAACAACAACTCCTGCACAAATTTAGAGAAACAAATAAAGCCTATTAAGGTTATAAAAAATGA | |
| | | | AGATTATATACATCTTAGCAAAGGAGAATACCTAATATATCGCCAAAAACCACTCTCATGGTATTGGATA | |
| | | | GACTTTAAGCAAACTACCTCTTTTGAAAGAAAGGTGCTAAAAATAAAAATAGTATGAAGATATTAAATAG | |
| | | | TTTAGAAGGTTATATTGACACCTATAATCCATGGAAAAATACATATGCACTTTTTAGAAGTTTACTTGGT | |
| | | | TTCTCAACATTACTAGTACTATTATTCAATAGTACTGATATTTTATTTAGTTATAGTGCAAATAATGTCA | |
| | | | CATGTGAAAATGTCTATATCCCTACCGCTTTTTGTTTTGCTAAAGAATATAGTATCAATTTTGAGATTAT | |
| | | | AAGATACTTAATGATTTTTATATTAACCTTAGTGGTTATAGGGTGGAGACCTAGATTTACCGGTTTATTT | |
| | | | CACTGGTATATTTGCTATAGTATTCAAACTTCAGCTTTAACTATCGATGGTGGAGAGCAAATTGCAACTG | |
| | | | TTCTTTCTTTTCTTATATTACCTGTTACATTATTAGATTCAAGGCGAAATCATTGGAATATAAAGAAAAA | |
| | | | CAATAATGAATCTTTCACAAAGAAGACAGTATTGTTTTATATAATGACAATAATTAAAATTCAAGTTTTT | |
| | | | ATCATTTATTTAAACGCAGCTTTAGAGCGATTGAAAAATAAAGAGTGGGCAGAAGGAACAGCAATTTACT | |
| | | | ATTTCTTTTCTGATCCGGTGTTTGGATTACCTGAATATCAACTTAACTTAATGAATCCACTACTTGAAAG | |
| | | | CAATTTTATTGTTGTCATCACTTGGTTAGTAACTATTTTTGAGTTGTTCTTAGCAGCAAGCATAATTTCA | |
| | | | AATATCAGAATAAAGAGAATTGCCCTTGTTTTGGGAATATTATTTCATATTGGGATAATATTCAGCATTG | |
| | | | GTATTGTAAGTTTTGGCTTGATCATGATATCAGCATTAATTATATATCTGCATCCTGTACAACAAAATAT | |
| | | | CACTATGAATTGGTGTTCTCCTTTATTTAAATATATATATGTAAAAGGAAAGAGAAATTTCAAAAGAATA | |
| | | | GGAGGTGAATCAGTCAAGTTTCTTACAAAATTGTTTCATAGCTAACATTTAGATAATGGAGAAATAACTT | |
| | | | AATGGAGGTATAATAATTTGAAAAGTAAATTACTTAGGCTATTGATTGTTTCCATGGTAACGATATTGGT | |
| | | | TTTTTCATTAGTAGGACTCTCTAAGGAGTCAAGTACATCTGCTAAAGAAAACCATACATTTTCTGGAGAA | |
| | | | GATTACTTTAGAGGACTTTTATTTGGACAAGGGGAAGTTGGTAAATTAATTTCAAACGATTTGGACCCTA | |
| | | | AACTCGTAAAAGAGGCAAATAGTACAGAAGGTAAAAAGTTAGTAAATGATGTAGTCAAATTTATAAAAAA | |
| | | | AGATCAACCACAATATATGGATGAATTGAAACAATCGATTGACAGCAAAGACCCTAAAAAACTCATTGAA | |
| | | | AATATGACCAAAGCAGACCAACTTATCCAAAAATATGCTAAGAAAAATGAAAACGTAAAATACTCTTCTA | |
| | | | ATAAAGTTACTCCATCTTGTGGGCTTTATGCCGTCTGTGTAGCAGCTGGATATTTATATGTTGTGGGCGT | |
| | | | TAACGCAGTTGCATTACAAACGGCTGCCGCAGTAACAACTGCAGTGTGGAAATACGTTGCCAAATATTCC | |
| | | | TCTTCAGCTTCTAATAATTCTGATTTAGAAGCGGCTGCTGCAAAAACCCTAAAATTGATTCATCAATAA | |
| 28 | Art- | DNA | ATGAAAAGAAACCAAAAAGAATGGGAATCTGTGAGTAAAAAAGGACTTATGAAGCCGGGAGGTACTTCGA | skfABC |
| | ificial | | TTGTGAAAGCTGCTGGCTGCATGGGCTGTTGGGCCTCGAAGAGTATTGCTATGACACGTGTTTGTGCACT | |
| | | | TCCGCATCCTGCTATGAGAGCTATTTAACATTTGAGAATAGGGAGTTGAGCGTATTTGCTTATACTCCTT | |
| | | | ATTTTCTCTTAAGGGGGATTTTATATGTCATATGATCGTGTTAAAGATTTTGATTTGCCAGAGTTAGCGG | |
| | | | TTCATTTACAGCCTCATGGTGCTGTAATGATTGATAGAAAAAGTATGTTTTATTTCAGACTCAGTGGACG | |
| | | | TGGAGCACAGTTGGCGTTTCTGTTATCAAAAAACAAAAATCTTCATAAAACGGCACGTATTTGGGAGATT | |
| | | | ATGAAAAAAGAAGAGATGAGTGCTGACCAATTAAAAGAAGAGCTTAGTGCCCATCCATTTACGGAGGCTT | |
| | | | GGACGGAAGGGCTGTTAGATCAGCCTTTACACGTTTCGGGTTCGCTAGATTCATATTTACCTATTAGTTG | |
| | | | TACCTTACAGTTGACAAATGCTTGTAATTTAAGCTGTTCGTTTTGCTATGCCAGCTCAGGTAAACCATAT | |
| | | | CCTGAGGAATTATCTAGTGAACAATGGATATTGGTTATGCAAAAGCTAGCAGCCCATGGAGTTGCTGATA | |
| | | | TTACGCTGACCGGGGGTGAAGCAAAGCTGATCAAAGGGTTTAAAGAATTAGTCGTTGTTGCAAGTTCGTT | |
| | | | GTTTACCAATGTGAATGTATTTAGTAATGGATTGAACTGGCGAGATGAAGAAGTTGAATTACTTAGTCAC | |
| | | | TTAGGCAATGTTTCTGTACAGATTAGTATTGATGGTATGGATAATACACATGACCAATTAAGAGGCAGAA | |
| | | | AAGGCGGCTTTAAAGAAAGTATGAATACCATTAAAAAATTATCAGAAGCAAACATTCCAGTGATTGTCGC | |
| | | | TATGACCATTAATGAGTCCAATGCTGATGAAGTATCAGATGTGGTGGAGCAATGTGCCAATGCGGGTGCT | |
| | | | TTTATCTTTCGTGCAGGAAAAACGTTATCTGTTGGACGTGCGACAGAAGGCTTTAAGGCTTTGGATATTG | |
| | | | ATTTTGAAGAAATGGTTCAAATACAGCTTAGGGAAGCACGTCATAAATGGGGAGATCGCCTGAACATAAT | |
| | | | TGATTGGGAGCACGAAGAGAGTTCGTTCACAACAGATTTTTGTACACCAGGATATCTTGCTTGGTATATA | |
| | | | AGAGCAGATGGATACGTAACTCCTTGCCAATTAGAAGATTTGCCGCTAGGGCATATTTTAGAAGATAGCA | |
| | | | TGGCTGACATTGGTTCACCTGCTCGTTTGCTTCAGTTAAAATGTGAAGCAAAAAATTGCAAATGTATAGG | |
| | | | GAAAATTGAGCTATCTGAACCGGACTTACCTTTTCAAAAAGAAGTCAAGGCAGGGATTCAGGAATGAATA | |
| | | | GTCTATCATTGGTGTTCTGGAGTATTTTAGCAGTTGTTGGATTACTGTTATTTATTAAATTCAAACCCCC | |
| | | | AACAATTGCTTCACTACTCTTAAGCAAAGATGAGGCAAAAGAAATAAGCATTCAATTTATAAAAGAGTTT | |
| | | | GTTGGGATAGATGTAGAGAACTGGGATTTTTATTCAGTATATTGGTATGACCACGATACAGTAAATAAAC | |
| | | | TTCATCACTTAGGCATACTTAAGAAAAATAGAAAGGTTTTATATGATGTTGGGTTGGTCGAATCATGGAG | |
| | | | AGTCCGTTTCGTTCACCAGAATCAATCATTTGTAGTTGGTGTCAATGCCAATCGAGAAATCACTTTTTTT | |
| | | | TATGCGGATGTTCCGAAAAAAACCCTTTCGGGGAAGTTTGAACAAGTTTCTCCAGAGACACTCAAGCAGA | |
| | | | GGTTAATGGCTTCACCTGATGGACTTTGGTCTAGAGCAAATATGACTGGTACTGGTAAAAAAGAGGAGGA | |
| | | | TTTTCGCGAGGTCAGTACTTATTGGTACATAGCGGAAGCGGGAGATATTCGGCTCAAAGTGACTGTTGAA | |
| | | | TTACAGGGCGGCCGAATTTCTTATATTGGTACTGAACAAGAAATACTAACAGATCAAATGAGTAAAGTCA | |
| | | | TTCGAGATGAACAAGTGGAATCGACATTCGGAGTATCTGGTATGCTGGGTTCAGCTTTAGCGATGATCCT | |
| | | | TGCGATTCTCATCCTTGTATTTATGGATGTGCAAACAAGCATAATCTTCAGTCTTGTTCTGGGTTTGTTG | |
| | | | ATTATAATATGCCAGTCATTGACGCTGAAAGAAGATATTCAATTAACAATTGTAAATGCTTATGATGCAA | |
| | | | GAATGAGTGTCAAAACGGTCAGTTTATTAGGTATTTTGTCTACACTTCTTACAGGATTATTAACAGGATT | |
| | | | TGTAGTATTTATATGTTCATTGGCAGGAAATGCGCTTGCTGGTGATTTTGGATGGAAAACGTTTGAACAA | |
| | | | CCAATAGTTCAGATTTTCTATGGAATAGGAGCAGGGCTCATTAGTTTAGGAGTGACTTCTCTGCTGTTTA | |
| | | | ACTTATTGGAGAAAAAGCAATATTTACGAATTTCACCTGAGCTTTCTAACCGAACTGTCTTTCTATCAGG | |
| | | | TTTTACCTTTAGGCAAGGATTGAATATGAGCATACAAAGTTCAATTGGAGAAGAGGTCATCTATCGGCTA | |
| | | | TTAATGATTCCAGTCATTTGGTGGATGAGTGGAAATATCCTCATCTCCATTATTGTATCTTCCTTTTTAT | |
| | | | GGGCGGTGATGCACCAAGTAACTGGATATGACCCAAGGTGGATACGTTGGCTGCATCTATTTATATTCGG | |
| | | | TTGCTTTCTGGGAGTTCTCTTCATCAAATTTGGTTTTATTTGTGTATTAGTAGCTCATTTCATTCATAAT | |
| | | | TTAGTACTCGTCTGTATGCCGCTGTGGCAGTTCAAGCTTCAGAAACATATGCATCATGATCAGCCAAAGC | |
| | | | ATACTTCACTCTAA | |
| 29 | Art- | DNA | ATGAAAAGAAACCAAAAAGAATGGGAATCTGTGAGTAAAAAAGGACTTATGAAGCCGGGAGGTACTTCGA | Seq. |
| | ificial | | TTGTGAAAGCTGCTGGCTGCATGGGCTGTTGGGCCTCGAAGAGTATTGCTATGACACGTGTTTGTGCACT | skfABC |
| | | | TCCGCATCCTGCTATGAGAGCTATTTAACATTTGAGAATAGGGAGTTGAGCGTATTTGCTTATACTCCTT | EFGH |
| | | | ATTTTCTCTTAAGGGGGATTTTATATGTCATATGATCGTGTTAAAGATTTTGATTTGCCAGAGTTAGCGG | |
| | | | TTCATTTACAGCCTCATGGTGCTGTAATGATTGATAGAAAAAGTATGTTTTATTTCAGACTCAGTGGACG | |
| | | | TGGAGCACAGTTGGCGTTTCTGTTATCAAAAAACAAAAATCTTCATAAAACGGCACGTATTTGGGAGATT | |
| | | | ATGAAAAAAGAAGAGATGAGTGCTGACCAATTAAAAGAAGAGCTTAGTGCCCATCCATTTACGGAGGCTT | |
| | | | GGACGGAAGGGCTGTTAGATCAGCCTTTACACGTTTCGGGTTCGCTAGATTCATATTTACCTATTAGTTG | |
| | | | TACCTTACAGTTGACAAATGCTTGTAATTTAAGCTGTTCGTTTTGCTATGCCAGCTCAGGTAAACCATAT | |
| | | | CCTGAGGAATTATCTAGTGAACAATGGATATTGGTTATGCAAAAGCTAGCAGCCCATGGAGTTGCTGATA | |
| | | | TTACGCTGACCGGGGGTGAAGCAAAGCTGATCAAAGGGTTTAAAGAATTAGTCGTTGTTGCAAGTTCGTT | |
| | | | GTTTACCAATGTGAATGTATTTAGTAATGGATTGAACTGGCGAGATGAAGAAGTTGAATTACTTAGTCAC | |
| | | | TTAGGCAATGTTTCTGTACAGATTAGTATTGATGGTATGGATAATACACATGACCAATTAAGAGGCAGAA | |
| | | | AAGGCGGCTTTAAAGAAAGTATGAATACCATTAAAAAATTATCAGAAGCAAACATTCCAGTGATTGTCGC | |
| | | | TATGACCATTAATGAGTCCAATGCTGATGAAGTATCAGATGTGGTGGAGCAATGTGCCAATGCGGGTGCT | |
| | | | TTTATCTTTCGTGCAGGAAAAACGTTATCTGTTGGACGTGCGACAGAAGGCTTTAAGGCTTTGGATATTG | |
| | | | ATTTTGAAGAAATGGTTCAAATACAGCTTAGGGAAGCACGTCATAAATGGGGAGATCGCCTGAACATAAT | |
| | | | TGATTGGGAGCACGAAGAGAGTTCGTTCACAACAGATTTTTGTACACCAGGATATCTTGCTTGGTATATA | |
| | | | AGAGCAGATGGATACGTAACTCCTTGCCAATTAGAAGATTTGCCGCTAGGGCATATTTTAGAAGATAGCA | |
| | | | TGGCTGACATTGGTTCACCTGCTCGTTTGCTTCAGTTAAAATGTGAAGCAAAAAATTGCAAATGTATAGG | |
| | | | GAAAATTGAGCTATCTGAACCGGACTTACCTTTTCAAAAAGAAGTCAAGGCAGGGATTCAGGAATGAATA | |
| | | | GTCTATCATTGGTGTTCTGGAGTATTTTAGCAGTTGTTGGATTACTGTTATTTATTAAATTCAAACCCCC | |
| | | | AACAATTGCTTCACTACTCTTAAGCAAAGATGAGGCAAAAGAAATAAGCATTCAATTTATAAAAGAGTTT | |
| | | | GTTGGGATAGATGTAGAGAACTGGGATTTTTATTCAGTATATTGGTATGACCACGATACAGTAAATAAAC | |
| | | | TTCATCACTTAGGCATACTTAAGAAAAATAGAAAGGTTTTATATGATGTTGGGTTGGTCGAATCATGGAG | |
| | | | AGTCCGTTTCGTTCACCAGAATCAATCATTTGTAGTTGGTGTCAATGCCAATCGAGAAATCACTTTTTTT | |
| | | | TATGCGGATGTTCCGAAAAAAACCCTTTCGGGGAAGTTTGAACAAGTTTCTCCAGAGACACTCAAGCAGA | |
| | | | GGTTAATGGCTTCACCTGATGGACTTTGGTCTAGAGCAAATATGACTGGTACTGGTAAAAAAGAGGAGGA | |
| | | | TTTTCGCGAGGTCAGTACTTATTGGTACATAGCGGAAGCGGGAGATATTCGGCTCAAAGTGACTGTTGAA | |
| | | | TTACAGGGCGGCCGAATTTCTTATATTGGTACTGAACAAGAAATACTAACAGATCAAATGAGTAAAGTCA | |
| | | | TTCGAGATGAACAAGTGGAATCGACATTCGGAGTATCTGGTATGCTGGGTTCAGCTTTAGCGATGATCCT | |
| | | | TGCGATTCTCATCCTTGTATTTATGGATGTGCAAACAAGCATAATCTTCAGTCTTGTTCTGGGTTTGTTG | |
| | | | ATTATAATATGCCAGTCATTGACGCTGAAAGAAGATATTCAATTAACAATTGTAAATGCTTATGATGCAA | |
| | | | GAATGAGTGTCAAAACGGTCAGTTTATTAGGTATTTTGTCTACACTTCTTACAGGATTATTAACAGGATT | |
| | | | TGTAGTATTTATATGTTCATTGGCAGGAAATGCGCTTGCTGGTGATTTTGGATGGAAAACGTTTGAACAA | |
| | | | CCAATAGTTCAGATTTTCTATGGAATAGGAGCAGGGCTCATTAGTTTAGGAGTGACTTCTCTGCTGTTTA | |
| | | | ACTTATTGGAGAAAAAGCAATATTTACGAATTTCACCTGAGCTTTCTAACCGAACTGTCTTTCTATCAGG | |
| | | | TTTTACCTTTAGGCAAGGATTGAATATGAGCATACAAAGTTCAATTGGAGAAGAGGTCATCTATCGGCTA | |
| | | | TTAATGATTCCAGTCATTTGGTGGATGAGTGGAAATATCCTCATCTCCATTATTGTATCTTCCTTTTTAT | |
| | | | GGGCGGTGATGCACCAAGTAACTGGATATGACCCAAGGTGGATACGTTGGCTGCATCTATTTATATTCGG | |
| | | | TTGCTTTCTGGGAGTTCTCTTCATCAAATTTGGTTTTATTTGTGTATTAGTAGCTCATTTCATTCATAAT | |
| | | | TTAGTACTCGTCTGTATGCCGCTGTGGCAGTTCAAGCTTCAGAAACATATGCATCATGATCAGCCAAAGC | |
| | | | ATACTTCACTCTAATGATTAGGAGAAGATTTGATGCAATTGATGCAAGTACAAAATTTAAGCAAATGCTA | |
| | | | TCGAAATGGTGATGGGGTTGAACATTTGTCTTTCTCAATTCAACGAGGAGAAATCGTGGCGCTATTGGGG | |
| | | | CCAAATGGAGCTGGAAAAACAACGACAATTCGATGTTTGACAGGTCTCTATAAGCCGGATAAAGGAGATA | |
| | | | TCCTCATTGAGGGTTCTCCTCCAGGAGATATAAATGTTCAAAAAAAAGTCGCGCTTATTCCTGATCAGCC | |
| | | | ATATTTGTATCCCGCTTTAACTGCTGCTGAACATATTCAGTTTAGGGCAAGAGGATATCATCCAGGTAAA | |
| | | | AAAGATGTAAAAGAAAGGGTTTATCATGCATTGAAGGAAGTGCACTTGGAGGAGAAAGCAAACCAATTGT | |
| | | | GTGGTCAGCTGTCAAGAGGCCAAAAGCAGCGGGTTGTCCTGGCGGGGGCGATTGTTCAAGACGCTTTGCT | |
| | | | GTATATATTAGACGAACCGACTGTGGGCCTTGATATCCCATCAAAACAGTGGCTCTCTAACTGGTTAAAA | |
| | | | ACTAAAACCGATCAAGGGTGTTCAGCATTTGTATCAACTCATAGTCTAGAGTTTGTTATAGAGACAGCAG | |
| | | | ACAGGGTGATATTAATTAGAGACGGCAAACTCATGCAGGATTTGTATGTTCCTCAATTCGAAGAACAAGC | |
| | | | GGAATGGAGAAAAGAAGTGATTCGTCTTCTAGGGGAATGGTCAGATGAATAATATCATTTCTTTTTTATG | |
| | | | GCTTCAGTCAAAAAGGCGTTTTGTAAGTCAAGGTCAGGAAAAAAAAATGCCTTTTTTGATCATGCTCCTT | |
| | | | TTTGTTGGGGCCATCGGATTTCAAGTAAGTTTTGTTTCTAGATCTACGACATGGGATATGAGTATTGCTG | |
| | | | GTTGGGTACTTACAGGTGTTTTTATCCTTTATACGGCATTTGGACTTTTTTCAAATCGATTACCAAGTCA | |
| | | | AATGGCAGATATTATATGGCTTTATGGCACTGCCACATCTTTTTCAAAAGTTGTGTATAGTGTTTTATTT | |
| | | | TTCAGCGTCACTTGGAAGGCGTTGCTTTGGATCATCTCAGCCATATTCGGTGATGTATTAATTGTGCTTC | |
| | | | TGTCTGGTGATCATATCAATTTATTAGGCCGATCCATAATTTTTGTAGGGCTCTTTTTTATCGCTGAAGT | |
| | | | GTGGTTGATGTCGGTCTCTTGTGCCAGAACAGTGAAGAAAATGAAAAGGGTATACGTTTTAGTCTTTCTT | |
| | | | CTAATGTTAGGCATTTACTCCATATGTCTTTATCGCTTCTTCTTTCTCCAACATTCATCTGGGATATGGG | |
| | | | AGAGCATTGCCCGTTTTATTAGTGGCGTTGGATTAGTGTTTGATACATTGTCACCGTTGTATGTGGTTGT | |
| | | | ATTTATTGGGATTATCACAGTCTCTTTTATGACAATTGCTTTTACGAGCCGTCAGGTAGAAATGAAGGAA | |
| | | | TCGTTGGTGAAAGAAGCTGAATTCTGGGAGGAATTTCAAGAACGTCAATTTGGTTCAGGTCAAATTATAC | |
| | | | AGAAACCAAAAACGACTTGGTGGGGCTTGCAAGGTCTAAATGGCATTTGGTCTTTTCTGTGGTTGGAACT | |
| | | | GTTGCTTTTTAAAAAATATTTATTTTTTCATAGCATTCATACGGTCATGCTCAGTGGCGTCTTTTATGTC | |
| | | | GTCATTTTCATGTATCCAGAATGGTTTTATCTTCTATTCTTTCTTATCGTCTCGGCAGTCATGTTAAGTT | |
| | | | CCTATTATTCAGGAATTGTCAGACATTCTCAATCAGGCACCCTTCATTTATTCCCCGGTGCCCTTTGGAA | |
| | | | AAAAATCATTATTCTAGAGCTGACGAATACAGTCTGGTTGTATATTCTTTACTGTGTTTCTATTACTTTT | |
| | | | ATGGCAGTTGGGAATTTAGTTTATTGGTATATATATGGCTTAGGGATATATATATGGTTCATGACAATAA | |
| | | | GGCTTTTTGCTTTTACCCATACAAACCGAAACGATATTAAGCTTTCATTGCCTCAATACTACAAGTCATT | |
| | | | TTTTATGGCATTAGGCCTGAGCGGCATTTGTTTGTATGTCATTCATTTATTGACTGCTGACTGGTATACA | |
| | | | TTAGTGGTGGTCGTCTGCATAGGGAGCCTAAGTTGGTGCCTGTTTTATCGTTTCAGATAGTGGTTATGTA | |
| | | | GAGTTATTGTTTTAAAACGAGGAGAGGGGCTCTTTTATGAATTCAAATGGTGATAAATTGTCGTTATCTG | |
| | | | TTCAGAATTTAGCGAATACAAATGAGATCACAATTGTTCAAGCGATAGGTGAGCTAAAGAAATCGGGAAA | |
| | | | AGATGCAATACCAGTTTTGGTTGAGGCCTTAAAAGAGGAAGGCTCTTTGTGTAATATTGCTGCAGCTGTT | |
| | | | TTAGGTGAGTTTGGGGAAGACGCGAGTGAAGCAGCGGAGGAGCTATCATGTTTATTAAAAAGTCATGCGG | |
| | | | AAGATACAAGAATGGCAGCGGCGATTTCATTAATGAGAATCGGGAAGCCCAGTCTGCCCTTTGTCATCAA | |
| | | | AATCGCTCAAGAAAGTGAAGGGCAATCGTGCTTTTGGGCATCTTGGTGTATTGCATGGATTGATCCGTCT | |
| | | | TGCATTGAACCTAAGATGTATAAATGTCTAAAATACGAGCATGAACATCCTTCAGGAATAGTTGCTCCGT | |
| | | | TTGCGGCTGAAGAGGCATTAGGAAAGCTGATTGCTTTTCAGCTGAAAGATAAGGAGGACTGAAAGGATGA | |
| | | | AAGATGAACAAATGTTGACTGAATGGCCAAGTCATTTACCATGGTTGAATCAATCACAGAATGATTTTAC | |
| | | | ATTTCCAAGTGACACATATCTTCTTCTTTATTTTTGGTCAATGAGCTGTCCAAACTGTCACCAATTAACA | |
| | | | GACAAAGTCCTTCAAGATATAAAGGATATGAATGTGAAAGTAATCGGAGTACATGTCCCCTATATAGAAG | |
| | | | AAGAGAAATCTATGGAGGTTGTCTTGACGTATGCTCTTGATAGGGGACTAGCTATTCCGATTGTATTAGA | |
| | | | CCAAAACTATGAGATCGTCACAACTTGTCACGTACAAGGCATCCCCAGCTTTTGTCTATTAAGTCAATAC | |
| | | | GGTCAGATCATTACTAAAACGATGGGAGATGTTGGTTGGGATAAGATGTTAAAAAAGATTGCAGGCTTGT | |
| | | | GA | |
| 30 | Art- | DNA | TTCTTCCCTCCGAACGAACCGCAGTTTTGTCCATATATGCCTTTTTATAACCTATGAGACAAGTTCCTTG | Seq. |
| | ificial | | AAAAGACGAAGAAAACATGTTTTACTTTGTAACAAATCAAAAATTTTTGTGCATAAGACTTGAAAGTCAA | PclpE- |
| | | | AGATAGTCAGAGTATACTATTAATCAAAGTTGGTCAAACAAACCGGCCTTTTTTAAAAATCAATTGGTCA | skfABC |
| | | | AAGATAGTCAAATATTCAGTCTGCTTTGAGCATATTGGTTTGAATGCCGTTAAGTTTGCCGTATACTAAT | EFGH |
| | | | AGTCAAAGAAGGTCAAACCCAATAGACCTTTTTATTCACTTTCATTGGTCAAAGATGATCAAATTATTAA | 30 |
| | | | GGAGGTTTTGGCAAATGAAAAGAAACCAAAAAGAATGGGAATCTGTGAGTAAAAAAGGACTTATGAAGCC | |
| | | | GGGAGGTACTTCGATTGTGAAAGCTGCTGGCTGCATGGGCTGTTGGGCCTCGAAGAGTATTGCTATGACA | |
| | | | CGTGTTTGTGCACTTCCGCATCCTGCTATGAGAGCTATTTAACATTTGAGAATAGGGAGTTGAGCGTATT | |
| | | | TGCTTATACTCCTTATTTTCTCTTAAGGGGGATTTTATATGTCATATGATCGTGTTAAAGATTTTGATTT | |
| | | | GCCAGAGTTAGCGGTTCATTTACAGCCTCATGGTGCTGTAATGATTGATAGAAAAAGTATGTTTTATTTC | |
| | | | AGACTCAGTGGACGTGGAGCACAGTTGGCGTTTCTGTTATCAAAAAACAAAAATCTTCATAAAACGGCAC | |
| | | | GTATTTGGGAGATTATGAAAAAAGAAGAGATGAGTGCTGACCAATTAAAAGAAGAGCTTAGTGCCCATCC | |
| | | | ATTTACGGAGGCTTGGACGGAAGGGCTGTTAGATCAGCCTTTACACGTTTCGGGTTCGCTAGATTCATAT | |
| | | | TTACCTATTAGTTGTACCTTACAGTTGACAAATGCTTGTAATTTAAGCTGTTCGTTTTGCTATGCCAGCT | |
| | | | CAGGTAAACCATATCCTGAGGAATTATCTAGTGAACAATGGATATTGGTTATGCAAAAGCTAGCAGCCCA | |
| | | | TGGAGTTGCTGATATTACGCTGACCGGGGGTGAAGCAAAGCTGATCAAAGGGTTTAAAGAATTAGTCGTT | |
| | | | GTTGCAAGTTCGTTGTTTACCAATGTGAATGTATTTAGTAATGGATTGAACTGGCGAGATGAAGAAGTTG | |
| | | | AATTACTTAGTCACTTAGGCAATGTTTCTGTACAGATTAGTATTGATGGTATGGATAATACACATGACCA | |
| | | | ATTAAGAGGCAGAAAAGGCGGCTTTAAAGAAAGTATGAATACCATTAAAAAATTATCAGAAGCAAACATT | |
| | | | CCAGTGATTGTCGCTATGACCATTAATGAGTCCAATGCTGATGAAGTATCAGATGTGGTGGAGCAATGTG | |
| | | | CCAATGCGGGTGCTTTTATCTTTCGTGCAGGAAAAACGTTATCTGTTGGACGTGCGACAGAAGGCTTTAA | |
| | | | GGCTTTGGATATTGATTTTGAAGAAATGGTTCAAATACAGCTTAGGGAAGCACGTCATAAATGGGGAGAT | |
| | | | CGCCTGAACATAATTGATTGGGAGCACGAAGAGAGTTCGTTCACAACAGATTTTTGTACACCAGGATATC | |
| | | | TTGCTTGGTATATAAGAGCAGATGGATACGTAACTCCTTGCCAATTAGAAGATTTGCCGCTAGGGCATAT | |
| | | | TTTAGAAGATAGCATGGCTGACATTGGTTCACCTGCTCGTTTGCTTCAGTTAAAATGTGAAGCAAAAAAT | |
| | | | TGCAAATGTATAGGGAAAATTGAGCTATCTGAACCGGACTTACCTTTTCAAAAAGAAGTCAAGGCAGGGA | |
| | | | TTCAGGAATGAATAGTCTATCATTGGTGTTCTGGAGTATTTTAGCAGTTGTTGGATTACTGTTATTTATT | |
| | | | AAATTCAAACCCCCAACAATTGCTTCACTACTCTTAAGCAAAGATGAGGCAAAAGAAATAAGCATTCAAT | |
| | | | TTATAAAAGAGTTTGTTGGGATAGATGTAGAGAACTGGGATTTTTATTCAGTATATTGGTATGACCACGA | |
| | | | TACAGTAAATAAACTTCATCACTTAGGCATACTTAAGAAAAATAGAAAGGTTTTATATGATGTTGGGTTG | |
| | | | GTCGAATCATGGAGAGTCCGTTTCGTTCACCAGAATCAATCATTTGTAGTTGGTGTCAATGCCAATCGAG | |
| | | | AAATCACTTTTTTTTATGCGGATGTTCCGAAAAAAACCCTTTCGGGGAAGTTTGAACAAGTTTCTCCAGA | |
| | | | GACACTCAAGCAGAGGTTAATGGCTTCACCTGATGGACTTTGGTCTAGAGCAAATATGACTGGTACTGGT | |
| | | | AAAAAAGAGGAGGATTTTCGCGAGGTCAGTACTTATTGGTACATAGCGGAAGCGGGAGATATTCGGCTCA | |
| | | | AAGTGACTGTTGAATTACAGGGCGGCCGAATTTCTTATATTGGTACTGAACAAGAAATACTAACAGATCA | |
| | | | AATGAGTAAAGTCATTCGAGATGAACAAGTGGAATCGACATTCGGAGTATCTGGTATGCTGGGTTCAGCT | |
| | | | TTAGCGATGATCCTTGCGATTCTCATCCTTGTATTTATGGATGTGCAAACAAGCATAATCTTCAGTCTTG | |
| | | | TTCTGGGTTTGTTGATTATAATATGCCAGTCATTGACGCTGAAAGAAGATATTCAATTAACAATTGTAAA | |
| | | | TGCTTATGATGCAAGAATGAGTGTCAAAACGGTCAGTTTATTAGGTATTTTGTCTACACTTCTTACAGGA | |
| | | | TTATTAACAGGATTTGTAGTATTTATATGTTCATTGGCAGGAAATGCGCTTGCTGGTGATTTTGGATGGA | |
| | | | AAACGTTTGAACAACCAATAGTTCAGATTTTCTATGGAATAGGAGCAGGGCTCATTAGTTTAGGAGTGAC | |
| | | | TTCTCTGCTGTTTAACTTATTGGAGAAAAAGCAATATTTACGAATTTCACCTGAGCTTTCTAACCGAACT | |
| | | | GTCTTTCTATCAGGTTTTACCTTTAGGCAAGGATTGAATATGAGCATACAAAGTTCAATTGGAGAAGAGG | |
| | | | TCATCTATCGGCTATTAATGATTCCAGTCATTTGGTGGATGAGTGGAAATATCCTCATCTCCATTATTGT | |
| | | | ATCTTCCTTTTTATGGGCGGTGATGCACCAAGTAACTGGATATGACCCAAGGTGGATACGTTGGCTGCAT | |
| | | | CTATTTATATTCGGTTGCTTTCTGGGAGTTCTCTTCATCAAATTTGGTTTTATTTGTGTATTAGTAGCTC | |
| | | | ATTTCATTCATAATTTAGTACTCGTCTGTATGCCGCTGTGGCAGTTCAAGCTTCAGAAACATATGCATCA | |
| | | | TGATCAGCCAAAGCATACTTCACTCTAATGATTAGGAGAAGATTTGATGCAATTGATGCAAGTACAAAAT | |
| | | | TTAAGCAAATGCTATCGAAATGGTGATGGGGTTGAACATTTGTCTTTCTCAATTCAACGAGGAGAAATCG | |
| | | | TGGCGCTATTGGGGCCAAATGGAGCTGGAAAAACAACGACAATTCGATGTTTGACAGGTCTCTATAAGCC | |
| | | | GGATAAAGGAGATATCCTCATTGAGGGTTCTCCTCCAGGAGATATAAATGTTCAAAAAAAAGTCGCGCTT | |
| | | | ATTCCTGATCAGCCATATTTGTATCCCGCTTTAACTGCTGCTGAACATATTCAGTTTAGGGCAAGAGGAT | |
| | | | ATCATCCAGGTAAAAAAGATGTAAAAGAAAGGGTTTATCATGCATTGAAGGAAGTGCACTTGGAGGAGAA | |
| | | | AGCAAACCAATTGTGTGGTCAGCTGTCAAGAGGCCAAAAGCAGCGGGTTGTCCTGGCGGGGGCGATTGTT | |
| | | | CAAGACGCTTTGCTGTATATATTAGACGAACCGACTGTGGGCCTTGATATCCCATCAAAACAGTGGCTCT | |
| | | | CTAACTGGTTAAAAACTAAAACCGATCAAGGGTGTTCAGCATTTGTATCAACTCATAGTCTAGAGTTTGT | |
| | | | TATAGAGACAGCAGACAGGGTGATATTAATTAGAGACGGCAAACTCATGCAGGATTTGTATGTTCCTCAA | |
| | | | TTCGAAGAACAAGCGGAATGGAGAAAAGAAGTGATTCGTCTTCTAGGGGAATGGTCAGATGAATAATATC | |
| | | | ATTTCTTTTTTATGGCTTCAGTCAAAAAGGCGTTTTGTAAGTCAAGGTCAGGAAAAAAAAATGCCTTTTT | |
| | | | TGATCATGCTCCTTTTTGTTGGGGCCATCGGATTTCAAGTAAGTTTTGTTTCTAGATCTACGACATGGGA | |
| | | | TATGAGTATTGCTGGTTGGGTACTTACAGGTGTTTTTATCCTTTATACGGCATTTGGACTTTTTTCAAAT | |
| | | | CGATTACCAAGTCAAATGGCAGATATTATATGGCTTTATGGCACTGCCACATCTTTTTCAAAAGTTGTGT | |
| | | | ATAGTGTTTTATTTTTCAGCGTCACTTGGAAGGCGTTGCTTTGGATCATCTCAGCCATATTCGGTGATGT | |
| | | | ATTAATTGTGCTTCTGTCTGGTGATCATATCAATTTATTAGGCCGATCCATAATTTTTGTAGGGCTCTTT | |
| | | | TTTATCGCTGAAGTGTGGTTGATGTCGGTCTCTTGTGCCAGAACAGTGAAGAAAATGAAAAGGGTATACG | |
| | | | TTTTAGTCTTTCTTCTAATGTTAGGCATTTACTCCATATGTCTTTATCGCTTCTTCTTTCTCCAACATTC | |
| | | | ATCTGGGATATGGGAGAGCATTGCCCGTTTTATTAGTGGCGTTGGATTAGTGTTTGATACATTGTCACCG | |
| | | | TTGTATGTGGTTGTATTTATTGGGATTATCACAGTCTCTTTTATGACAATTGCTTTTACGAGCCGTCAGG | |
| | | | TAGAAATGAAGGAATCGTTGGTGAAAGAAGCTGAATTCTGGGAGGAATTTCAAGAACGTCAATTTGGTTC | |
| | | | AGGTCAAATTATACAGAAACCAAAAACGACTTGGTGGGGCTTGCAAGGTCTAAATGGCATTTGGTCTTTT | |
| | | | CTGTGGTTGGAACTGTTGCTTTTTAAAAAATATTTATTTTTTCATAGCATTCATACGGTCATGCTCAGTG | |
| | | | GCGTCTTTTATGTCGTCATTTTCATGTATCCAGAATGGTTTTATCTTCTATTCTTTCTTATCGTCTCGGC | |
| | | | AGTCATGTTAAGTTCCTATTATTCAGGAATTGTCAGACATTCTCAATCAGGCACCCTTCATTTATTCCCC | |
| | | | GGTGCCCTTTGGAAAAAAATCATTATTCTAGAGCTGACGAATACAGTCTGGTTGTATATTCTTTACTGTG | |
| | | | TTTCTATTACTTTTATGGCAGTTGGGAATTTAGTTTATTGGTATATATATGGCTTAGGGATATATATATG | |
| | | | GTTCATGACAATAAGGCTTTTTGCTTTTACCCATACAAACCGAAACGATATTAAGCTTTCATTGCCTCAA | |
| | | | TACTACAAGTCATTTTTTATGGCATTAGGCCTGAGCGGCATTTGTTTGTATGTCATTCATTTATTGACTG | |
| | | | CTGACTGGTATACATTAGTGGTGGTCGTCTGCATAGGGAGCCTAAGTTGGTGCCTGTTTTATCGTTTCAG | |
| | | | ATAGTGGTTATGTAGAGTTATTGTTTTAAAACGAGGAGAGGGGCTCTTTTATGAATTCAAATGGTGATAA | |
| | | | ATTGTCGTTATCTGTTCAGAATTTAGCGAATACAAATGAGATCACAATTGTTCAAGCGATAGGTGAGCTA | |
| | | | AAGAAATCGGGAAAAGATGCAATACCAGTTTTGGTTGAGGCCTTAAAAGAGGAAGGCTCTTTGTGTAATA | |
| | | | TTGCTGCAGCTGTTTTAGGTGAGTTTGGGGAAGACGCGAGTGAAGCAGCGGAGGAGCTATCATGTTTATT | |
| | | | AAAAAGTCATGCGGAAGATACAAGAATGGCAGCGGCGATTTCATTAATGAGAATCGGGAAGCCCAGTCTG | |
| | | | CCCTTTGTCATCAAAATCGCTCAAGAAAGTGAAGGGCAATCGTGCTTTTGGGCATCTTGGTGTATTGCAT | |
| | | | GGATTGATCCGTCTTGCATTGAACCTAAGATGTATAAATGTCTAAAATACGAGCATGAACATCCTTCAGG | |
| | | | AATAGTTGCTCCGTTTGCGGCTGAAGAGGCATTAGGAAAGCTGATTGCTTTTCAGCTGAAAGATAAGGAG | |
| | | | GACTGAAAGGATGAAAGATGAACAAATGTTGACTGAATGGCCAAGTCATTTACCATGGTTGAATCAATCA | |
| | | | CAGAATGATTTTACATTTCCAAGTGACACATATCTTCTTCTTTATTTTTGGTCAATGAGCTGTCCAAACT | |
| | | | GTCACCAATTAACAGACAAAGTCCTTCAAGATATAAAGGATATGAATGTGAAAGTAATCGGAGTACATGT | |
| | | | CCCCTATATAGAAGAAGAGAAATCTATGGAGGTTGTCTTGACGTATGCTCTTGATAGGGGACTAGCTATT | |
| | | | CCGATTGTATTAGACCAAAACTATGAGATCGTCACAACTTGTCACGTACAAGGCATCCCCAGCTTTTGTC | |
| | | | TATTAAGTCAATACGGTCAGATCATTACTAAAACGATGGGAGATGTTGGTTGGGATAAGATGTTAAAAAA | |
| | | | GATTGCAGGCTTGTGA | |
| 31 | Art- | DNA | TTCTTCCCTCCGAACGAACCGCAGTTTTGTCCATATATGCCTTTTTATAACCTATGAGACAAGTTCCTTG | Seq. |
| | ificial | | AAAAGACGAAGAAAACATGTTTTACTTTGTAACAAATCAAAAATTTTTGTGCATAAGACTTGAAAGTCAA | PclpE- |
| | | | AGATAGTCAGAGTATACTATTAATCAAAGTTGGTCAAACAAACCGGCCTTTTTTAAAAATCAATTGGTCA | skfABC |
| | | | AAGATAGTCAAATATTCAGTCTGCTTTGAGCATATTGGTTTGAATGCCGTTAAGTTTGCCGTATACTAAT | EFGH |
| | | | AGTCAAAGAAGGTCAAACCCAATAGACCTTTTTATTCACTTTCATTGGTCAAAGATGATCAAATTATTAA | |
| | | | GGAGGTTTTGGCAAATGACTATATGTTTCCTATTATTTTCTTCTTATTACTTTAGCAATATTTCACCTCA | |
| | | | GAATCCACTGTTCAAAAAAAATTTTTTGCAACAATTGTCTCCCCAAGGCTTTGGCTTTTATAGTAAAAGC | |
| | | | CCTACAGAAGAAAACATTTCATTTCACACAAAAGAAAATTTAAAGTTACCTAATGCACTTCCCAATAATT | |
| | | | TTTTTGGGATAAAAAGAGAAGGAAGAGTTCAGGCAATAGAATTAGGCAAAATTGTAGAGAATATCGATCC | |
| | | | AAAGAATTGGAAAACTTGTGAAAACAACAACTCCTGCACAAATTTAGAGAAACAAATAAAGCCTATTAAG | |
| | | | GTTATAAAAAATGAAGATTATATACATCTTAGCAAAGGAGAATACCTAATATATCGCCAAAAACCACTCT | |
| | | | CATGGTATTGGATAGACTTTAAGCAAACTACCTCTTTTGAAAGAAAGGTGCTAAAAATAAAAATAGTATG | |
| | | | AAGATATTAAATAGTTTAGAAGGTTATATTGACACCTATAATCCATGGAAAAATACATATGCACTTTTTA | |
| | | | GAAGTTTACTTGGTTTCTCAACATTACTAGTACTATTATTCAATAGTACTGATATTTTATTTAGTTATAG | |
| | | | TGCAAATAATGTCACATGTGAAAATGTCTATATCCCTACCGCTTTTTGTTTTGCTAAAGAATATAGTATC | |
| | | | AATTTTGAGATTATAAGATACTTAATGATTTTTATATTAACCTTAGTGGTTATAGGGTGGAGACCTAGAT | |
| | | | TTACCGGTTTATTTCACTGGTATATTTGCTATAGTATTCAAACTTCAGCTTTAACTATCGATGGTGGAGA | |
| | | | GCAAATTGCAACTGTTCTTTCTTTTCTTATATTACCTGTTACATTATTAGATTCAAGGCGAAATCATTGG | |
| | | | AATATAAAGAAAAACAATAATGAATCTTTCACAAAGAAGACAGTATTGTTTTATATAATGACAATAATTA | |
| | | | AAATTCAAGTTTTTATCATTTATTTAAACGCAGCTTTAGAGCGATTGAAAAATAAAGAGTGGGCAGAAGG | |
| | | | AACAGCAATTTACTATTTCTTTTCTGATCCGGTGTTTGGATTACCTGAATATCAACTTAACTTAATGAAT | |
| | | | CCACTACTTGAAAGCAATTTTATTGTTGTCATCACTTGGTTAGTAACTATTTTTGAGTTGTTCTTAGCAG | |
| | | | CAAGCATAATTTCAAATATCAGAATAAAGAGAATTGCCCTTGTTTTGGGAATATTATTTCATATTGGGAT | |
| | | | AATATTCAGCATTGGTATTGTAAGTTTTGGCTTGATCATGATATCAGCATTAATTATATATCTGCATCCT | |
| | | | GTACAACAAAATATCACTATGAATTGGTGTTCTCCTTTATTTAAATATATATATGTAAAAGGAAAGAGAA | |
| | | | ATTTCAAAAGAATAGGAGGTGAATCAGTCAAGTTTCTTACAAAATTGTTTCATAGCTAACATTTAGATAA | |
| | | | TGGAGAAATAACTTAATGGAGGTATAATAATTTGAAAAGTAAATTACTTAGGCTATTGATTGTTTCCATG | |
| | | | GTAACGATATTGGTTTTTTCATTAGTAGGACTCTCTAAGGAGTCAAGTACATCTGCTAAAGAAAACCATA | |
| | | | CATTTTCTGGAGAAGATTACTTTAGAGGACTTTTATTTGGACAAGGGGAAGTTGGTAAATTAATTTCAAA | |
| | | | CGATTTGGACCCTAAACTCGTAAAAGAGGCAAATAGTACAGAAGGTAAAAAGTTAGTAAATGATGTAGTC | |
| | | | AAATTTATAAAAAAAGATCAACCACAATATATGGATGAATTGAAACAATCGATTGACAGCAAAGACCCTA | |
| | | | AAAAACTCATTGAAAATATGACCAAAGCAGACCAACTTATCCAAAAATATGCTAAGAAAAATGAAAACGT | |
| | | | AAAATACTCTTCTAATAAAGTTACTCCATCTTGTGGGCTTTATGCCGTCTGTGTAGCAGCTGGATATTTA | |
| | | | TATGTTGTGGGCGTTAACGCAGTTGCATTACAAACGGCTGCCGCAGTAACAACTGCAGTGTGGAAATACG | |
| | | | TTGCCAAATATTCCTCTTCAGCTTCTAATAATTCTGATTTAGAAGCGGCTGCTGCAAAAACCCTAAAATT | |
| | | | GATTCATCAATAA | |
| 32 | Bacillus | DNA | ATGAAAAAATTTATTGCTTTACTGTTCTTTATATTGCTTCTTTCGGGTTGCGGGGT | lytA |
| | subtilis | | TAATAGTCAAAAGAGTCAAGGTGAAGATGTATCGCCAGACAGTAACATTGAAACAA | |
| | | | AAGAAGGTACTTATGTAGGGTTAGCTGATACTCATACAATAGAAGTAACAGTAGAT | |
| | | | AATGAGCCGGTTAGTCTTGATATCACTGAAGAATCGACAAGTGATCTTGATAAGTT | |
| | | | TAACAGTGGAGATAAGGTCACGATTACATATGAAAAAAATGATGAGGGTCAGCTTC | |
| | | | TGTTAAAAGATATTGAACGTGCCAACTAA | |
| 33 | Bacillus | DNA | TCAATAACTTGTCAGAGTTGTACCTGGATAGTAAAATTTCAAAATTGAACTATATG | lytB |
| | subtilis | | AATCTCCAGCTTCTGCTCTTGCTTTTGCTCCATATTGACTCATTCCGATCCCGTGA | |
| | | | CCATATCCTTTACCGCTGATTGTGTACTTTGAAGTATCTTTTTTTACAGTCACATA | |
| | | | AGTACTTTTAAATACAGTCGCTCCAATCATTGTTCTTAACTCACTTGTTGGTACAC | |
| | | | TAATGGTTGTAATTTTGCTCAAGTTATAGGAGCCAGTACTGCTTTTAACAAAATAC | |
| | | | TTCACTTTCATCGATGCTGTTTTTGCACGCTGCCCTTGTGTCGTTCCACTAAAGCT | |
| | | | TAAATCGTCAATACTTGCAATTTTGACAGAATCAGCGCTTGTTTCTTTATTTTTCA | |
| | | | AGATCCAGTTCTTAACTCCGGAAAGTCTTGCAGAATCAGTTTCAGTCGCTGAAGAC | |
| | | | CACCAAGAAGAAGGCTTCGTTAAGTCCAAAGATTTCGTATCTAGCTGTTGTTTTGA | |
| | | | CAAAGTAAGCGTCCAGCCTATTTGAGGATCCTTTGTATCTTTTTTTGCAATCAAGT | |
| | | | AAGGTACACTTGACGACCACACTTCATTACTAGCTTCTGTGTAACCACCATTACTG | |
| | | | GAAGAATAAGCTGCAGTTATAAGAGATCCATTATATTTCAGAACTTTCCCCTTCGT | |
| | | | TTGCTCTACCGCTTTATTGGTATTCGAATTCCAACTGTAGCCACCATAAACTTGAA | |
| | | | AAGCGGTTGTATCAGGTACAGTAGTTCCTGTTTTTGTTATTGAGTAGGTTCTTGCA | |
| | | | GCGACAGTCTGTGCTTTAAGAGCCTCAAGTGACCAGCTAGCAGGCATTTCATTCGG | |
| | | | AATTACTCCTTTTAAGTAATCCTCAAATGGAATATTCTCATTTACCGGTCGGATAT | |
| | | | ATTTAGTAGACTCAATAGAGAAATTAACCGTGCCAAGATACTGCTTTCCATCAAGA | |
| | | | CTAATTCTGTTTGAAGTAGAATAGTTTTCTGGTTTAATTCTTAGGGAGTTTCCGTA | |
| | | | AGTTTTGATGTTTTCAAGGTTTAATTTTCCGCTGTTGATTTTTAGATTATAGGTGC | |
| | | | CACCGTCAGCTAAATAAAATTCATCAGCAAGTGAATTCTCAACTTCAGCAGAAATT | |
| | | | GAACTGGTACTTCCAATAAAGTCGTATGCATATGTGGCTTTATCTTTCGTTATACT | |
| | | | TTTAGCCGCAGCCGGAACGCTATCTTGTTTAACAAATAAAATTTGGCTGTTTTTCT | |
| | | | TCGAGGCAAGCGAAGCGCCTATCAATACGTCTGCATATTTTGTCCCGTTCGTCATT | |
| | | | ACGACTTTATCAGCCTTCAACTTTAGTTGCTTAATTATATTTGCCGTAAGTTCATA | |
| | | | TCTGGTTGATCCAGGAATCCTTTGCACAGTTGAAGTTTTTTTAATTTGGTTTTCTA | |
| | | | CACTATCACTGACGCTAAATGAACTGCCTATAATGATGACTTGTTTTGGCAAATCA | |
| | | | TAATCCGGAAGCTTGTCTTTCTCTGTAAGCAAAATAGGGTATCCGTGTGCTGCTGC | |
| | | | ATATGGAGCAATGGCTAAAGCGTCTTGAAATACTCTTCCGGTTACAACGATCGCTT | |
| | | | TATCATAGCTGCCCATTTGCTTTGCAATATTTTCTGATAAGACATATCTATTTTTT | |
| | | | CCGCTTATCCTTTTCACAGCGCCATATGATTTCAGCTTGTTTTCTACGTCTTTTGA | |
| | | | AATGCTTCGCGCTCCCCCAATGATTAAAATATTATCAGGGTTAAACTTAGCAATTT | |
| | | | GTCTTTCAGTAGTTTTTGTTAACGTGTCAGGCTGAGTAAATAAAACTGGTGCGTTC | |
| | | | AGTTTTTTCGCCAATGGAATAACAGGCAATGCATCAATAAAAATATCTCGGTTCAC | |
| | | | CAATATCACTGTATTAGGATTTTTCCACTGGCTGTTGGATGCAAGAGTCGCAGTTT | |
| | | | CGTATCGTGAAGCGCCTGCAAAACGATCCGTAACAGCAACATTATCTCCAGTTACT | |
| | | | TTATAAAATCCTGTTGGCGATAGACTGATGCTAGATTTATTTCCAATATAATTTAA | |
| | | | CAATTTAACTGAGATGTTTGAGTCTGCTGCAAAAGAAACTGATGGAATCAATAATA | |
| | | | AGATTGCAGCAAGTGAACACACTATCAATTGTTTGCAAGATTTCAA | |
| 34 | Bacillus | DNA | TTATCTGTAATAAGATACTGTGCCGTCATGAATAGCTTGTGCAGCTTTATCTTTAT | lytC |
| | subtilis | | AAACCGCTTGCTTCAATTTACTTGCATCTGATGCATTAGTGATAAAGGCAGTTTCA | |
| | | | ACTAAAACACTCGGCATTTTAGAATATTTAATAACATAGAAAGCAGCTGTTTTTAC | |
| | | | TCCCCGGTCTCTCGTTCCAAGATTAGCCGCTAACTTTGGTTGAATTTGTTCAGCCA | |
| | | | GTCTCTTGCTATTTGCAGCTTGATATGTTGTATCGTAGTACGTCTCACTTCCATTT | |
| | | | GGTGATGAGCTATCATTAGCATTTGCATGTATACTGAGAAATAAATCTGCTTGTGC | |
| | | | AGAAGCTGCTTTATTTACTCTCTCCTGTAAAGAATAAAAAGTATCATTAGATCTTG | |
| | | | ACAGTACTGGAAGAGCACCTGAAGCATTTAGCTTTGTATTGACTCTTTTCGCTATA | |
| | | | TCAAGGTTGACTTCTTTCTCAAGGAGTCCATTGCCGATTGCTCCTGAATCTTGATC | |
| | | | ACCGTGACCCGGATCAATAAAGATTGTTTCACCTACAACTGGATTCTTTAGCTGAT | |
| | | | TAGCAACCTTTGTGCTTACGGCAGGAGTGTTTCCGATAATCATAAAGTTTGACATG | |
| | | | TTTTTACTTCCAATAATTTTACGGGCTCCTGTAGATAAATTTTCACCATTTGTAAG | |
| | | | AATAAGAGATTGCTTCTTCTTAGCTGCCAGTGTAGCTCCTGCAATAGAGTCAGGGT | |
| | | | AGCTGAATCCATTGCTTACATATACGGTGCTTGTTGATAAATTAAGTTTTTGTACG | |
| | | | ATATTTGCAGCAAGCTCATATCTGTTTGAACCGCTAATTCTTGTAGGAGAAGGTAA | |
| | | | CTTGTTGTATACCGTATTGCTGATACTTCCAGTGCCTCCTACAACAACGGTACTCG | |
| | | | AAATTCCCTTATCTTTTATCACAGACGTAGTCGCACTATTTATAGATGTTTTATTT | |
| | | | GTAAAAAGAATTGGATACCCGTTTTTCGCTGCATAAGGGATGACGGCCGGAGCGTC | |
| | | | TGCATATAAGAAGCCGTTCAAAATAACAGCTTTTGAAGTCGCACCCATCGCTTTTG | |
| | | | CCACCCGTGCAGCCGTATCATAACGGTTGCTTCCTGCAATTCGTTTAATACTTATC | |
| | | | CCCAAGCTTTTAATCTGGTTAGCAGTGTTAGAAGAAACAGCAGGTGTTCCGCCTAC | |
| | | | AATAATTACATTTTTAGTCTGCATCTCTTTCAATCTTGTTTTCGTTTCATATGAAA | |
| | | | GCTTATCAGAATTAGTGTAAAGCAATGGCGCATTCTTCTGGTAAGCAAGAGGTGCT | |
| | | | GCTGAAATAGCATCTGCATAGGAACTCCCACCAACAATTACAGCTGTACTTGCTGT | |
| | | | TGAATACATTTGCTTTGATATTTGTACAGCAGTGCCGTATCTATTGCTTCCCCCAA | |
| | | | CTCTTTTCACTGAGTTATCGGCCAAAGCTGTTGGCACAAAAAGTATGAGCCCCAGA | |
| | | | AAACACATTGTTAGGACTTTTATATAAGAACGCAA | |
| 35 | Bacillus | DNA | ATGAAAAAGCAAATCATTACAGCTACGACAGCAGTTGTTTTAGGATCGACGTTATT | lytE |
| | subtilis | | TGCAGGAGCGGCATCTGCACAAAGCATTAAGGTGAAAAAAGGCGACACGTTATGGG | |
| | | | ATCTTTCAAGAAAATACGACACAACGATCAGTAAAATTAAATCAGAGAACCACCTT | |
| | | | CGTTCAGACATTATTTATGTGGGACAAACTTTATCGATTAACGGCAAATCTACAAG | |
| | | | TTCAAAAAGCAGCAGTTCTTCTTCCTCTTCTTCTACATACAAAGTAAAGAGCGGGG | |
| | | | ACAGCCTTTGGAAAATTTCAAAAAAATACGGCATGACAATCAATGAACTGAAGAAG | |
| | | | CTGAATGGCTTAAAATCAGATTTGCTTCGTGTTGGACAAGTCCTGAAACTGAAAGG | |
| | | | TTCAACTAGTTCAAGCAGCTCCAGCTCATCAAAAGTGTCATCGTCTTCAACTTCTA | |
| | | | CTTATAAAGTGAAGAGCGGAGACAGCCTTTCTAAAATTGCGAGCAAATACGGCACT | |
| | | | ACGGTTAGCAAATTAAAAAGCTTAAACGGCTTAAAATCAGATGTAATCTATGTCAA | |
| | | | CCAAGTATTGAAGGTGAAAGGAACAAGCACAAGCAGCTCAAAACCTGCTTCATCTT | |
| | | | CATCGTCTTCAAGCAGCAAAACGTCATCTACATCACTTAATGTGAGCAAGCTGGTT | |
| | | | TCTGATGCAAAAGCGTTAGTCGGAACGCCATATAAATGGGGCGGAACGACAACTTC | |
| | | | AGGCTTTGACTGCAGCGGATTCATTTGGTACGTACTGAATAAACAAACAAGTGTGG | |
| | | | GCAGAACAAGCACTGCAGGATACTGGAGTTCTATGAAGAGCATTGCCAGCCCGTCT | |
| | | | GTTGGTGATTTCGTCTTCTTCACAACATATAAATCCGGCCCTTCTCACATGGGGAT | |
| | | | TTACATTGGAAACAACAGTTTCATTCATGCAGGATCTGACGGCGTACAAATCAGCA | |
| | | | GCCTGAACAACAGCTACTGGAAGCCTCGTTACCTCGGTGCGAAAAGATTCTAA | |
| 36 | Bacillus | DNA | ATGTCAGTTTTCACTAATAGCTACATTCCAGTCAATAAGTATACTAGACCAGGTTT | blyA |
| | subtilis | | GAAATTACAGGGTGTGAAAAAATGCGTCCTACACTATACAGCCAATCCGGGTGCAG | |
| | | | GTGCAGACAATCATCGAAGATACTTCAGTAATGCACAAGTTTATGCATCAGCTCAC | |
| | | | ATTTTCGTAGATAAGGCTGAAGCAATTTGTATCATTCCATTAAATGAAGTAGCTTA | |
| | | | CCATGCAAATGATATTCAGCAAAGAGATAGTGCCGGAAATCCTTATCGAGGAGTTG | |
| | | | CTGCGCTGAAACCTAACGCTAACTTTCTTTCTATTGGAGTTGAAATGTGCCTTGAA | |
| | | | AAAGACGGTTCATTCCATTCAGATACAGTTGAAAGAACTGAGGATGTGTTCGTTGA | |
| | | | ATTATGTAATAAGTTTGGTTTAGATCCTATTGATGATATTGTTCGTCATTATGACA | |
| | | | TCACCCATAAGAATTGCCCTGCACCATGGGTATCTAACAGCCAAAAATTTGTAGAC | |
| | | | TTTAAAAATCGAGTAAAGGCAAAAATGTCAGGCAAATCTGTTTCAAAAGCTTCTCC | |
| | | | AACTAAACCAACAACCTCCTCCCCTTCCTCTTCATCAGCAGTAAGTGGTTCACTAA | |
| | | | AATCAAAAGTTGACGGACTTCGCTTCTATTCAAAACCATCTTGGGAAGATAAAGAT | |
| | | | GTTGTCGGCACAGTAAATAAAGGCATCGGATTTCCTACAGTTGTAGAGAAAGTTAA | |
| | | | AGTTGGATCTGCCTATCAATACAAAGTTAAGAACTCAAAAGGCACTACATATTACA | |
| | | | TCACTGCTTCTGACAAATATGTTGATGTTACAGGATCAGTTAAAACCTCTTCCTCT | |
| | | | GCCCCAAAAACAACATCAACTTCTTCAAGTTCCTCATCTATTAAATCCGTAGGAAA | |
| | | | AATCAAAATTGTCGGTGTATCAAGCGCTGCAATCGTAATGGACAAGCCTGATCGAA | |
| | | | ATAGTTCTAAAAATATTGGCACAGTTAAGCTTGGAAGCACTATTTCAATTTCTGGT | |
| | | | TCAGTTAAAGGTAAAAACAATTCCAATGGCTACTGGGAAGTTATTTATAAAGGTAA | |
| | | | ACGTGGATATATCTCGGGACAGTTTGGATCAACAATCTAA | |
| 37 | Bacillus | DNA | ATGGAAATGGATATAACACAATATTTAAGTACCCAGGGGCCATTTGCTGTTTTATT | bhlA |
| | subtilis | | TTGTTGGCTACTTTTCTACGTAATGAAAACTAGTAAGGAAAGAGAGTCGAAACTTT | |
| | | | ATAATCAAATCGATTCTCAAAACGAAGTACTGGGTAAATTCAGTGAAAAGTACGAT | |
| | | | GTTGTAATTGAAAAGCTAGATAAAATCGAACAAAATTTTAAGTAG | |
| 38 | Bacillus | DNA | ATGTTTGAGAATATTGATAAAGGCACAATTGTTAGGACTCTTTTGCTCGCAATAGC | bhlB |
| | subtilis | | TTTACTCAATCAAATAATGGTGATGCTGGGTAAAGCAGCATTCATCATTAACGAAG | |
| | | | AGGACATAAATCATTTATATGATTGTTTATATACAATTTTCACTATCGTCTTCACA | |
| | | | ACCAGTACTACTACCGCAGCATGGTTCAAAAACAATTACATAACTGCAAAAGGAAA | |
| | | | AAAACAAAAACAAGTTCTAAAAAAAGAGAACTTGTTTAAATAG | |
| 39 | Bacillus | DNA | ATGGCCATTAAAGTTGTAAAGAATCTAGTCTCTAAATCAAAGTATGGATTGAAATG | cwlA |
| | subtilis | | TCCTAATCCAATGAAAGCTGAATATATCACTATTCATAACACTGCGAATGATGCTT | |
| | | | CAGCAGCCAATGAGATTTCTTACATGAAGAATAACTCTAGCTCAACAAGTTTTCAC | |
| | | | TTTGCAGTAGACGATAAACAAGTCATTCAAGGTATTCCAACGAATCGTAACGCTTG | |
| | | | GCACACAGGAGATGGAACAAACGGTACAGGGAATCGCAAGTCTATTGGTGTCGAAA | |
| | | | TTTGTTATAGCAAGTCAGGAGGGGTACGATATAAGGCAGCGGAAAAGCTTGCTATT | |
| | | | AAGTTTGTGGCTCAGCTACTTAAAGAACGTGGATGGGGTATTGATCGAGTCCGCAA | |
| | | | ACATCAAGACTGGAATGGTAAGTATTGCCCGCACCGCATTTTGTCAGAGGGAAGAT | |
| | | | GGATTCAAGTTAAGACTGCAATTGAAGCAGAATTGAAAAAGTTGGGCGGAAAAACA | |
| | | | AACTCAAGCAAAGCAAGTGTAGCTAAAAAGAAAACAACAAACACAAGCAGCAAAAA | |
| | | | AACGTCATATGCGCTACCATCCGGTATTTTTAAAGTGAAGAGCCCAATGATGAGAG | |
| | | | GGGAAAAGGTAACACAAATTCAAAAAGCACTGGCTGCACTATACTTTTACCCGGAT | |
| | | | AAAGGAGCAAAAAACAACGGCATTGACGGCGTGTATGGTCCGAAAACAGCAGATGC | |
| | | | AATTAGACGATTCCAGTCTATGTACGGGCTTACTCAAGACGGTATTTACGGACCAA | |
| | | | AAACGAAAGCGAAACTTGAAGCTCTCTTGAAGTAA | |
| 40 | Bacillus | Protein | MKKFIALLFFILLLSGCGVNSQKSQGEDVSPDSNIETKEGTYVGLADTHTIEVTVD | lytA |
| | subtilis | | NEPVSLDITEESTSDLDKFNSGDKVTITYEKNDEGQLLLKDIERAN | |
| 41 | Bacillus | | MKSCKQLIVCSLAAILLLIPSVSFAADSNISVKLLNYIGNKSSISLSPTGFYKVTG | lytB |
| | subtilis | | DNVAVTDRFAGASRYETATLASNSQWKNPNTVILVNRDIFIDALPVIPLAKKLNAP | |
| | | | VLFTQPDTLTKTTERQIAKFNPDNILIIGGARSISKDVENKLKSYGAVKRISGKNR | |
| | | | YVLSENIAKQMGSYDKAIVVTGRVFQDALAIAPYAAAHGYPILLTEKDKLPDYDLP | |
| | | | KQVIIIGSSFSVSDSVENQIKKTSTVQRIPGSTRYELTANIIKQLKLKADKVVMTN | |
| | | | GTKYADVLIGASLASKKNSQILFVKQDSVPAAAKSITKDKATYAYDFIGSTSSISA | |
| | | | EVENSLADEFYLADGGTYNLKINSGKLNLENIKTYGNSLRIKPENYSTSNRISLDG | |
| | | | KQYLGTVNFSIESTKYIRPVNENIPFEDYLKGVIPNEMPASWSLEALKAQTVAART | |
| | | | YSITKTGTTVPDTTAFQVYGGYSWNSNTNKAVEQTKGKVLKYNGSLITAAYSSSNG | |
| | | | GYTEASNEVWSSSVPYLIAKKDTKDPQIGWTLTLSKQQLDTKSLDLTKPSSWWSSA | |
| | | | TETDSARLSGVKNWILKNKETSADSVKIASIDDLSFSGTTQGQRAKTASMKVKYFV | |
| | | | KSSTGSYNLSKITTISVPTSELRTMIGATVFKSTYVTVKKDTSKYTISGKGYGHGI | |
| | | | GMSQYGAKARAEAGDSYSSILKFYYPGTTLTSY | |
| 42 | Bacillus | Protein | MRSYIKVLTMCFLGLILFVPTALADNSVKRVGGSNRYGTAVQISKQMYSTASTAVI | lytC |
| | subtilis | | VGGSSYADAISAAPLAYQKNAPLLYTNSDKLSYETKTRLKEMQTKNVIIVGGTPAV | |
| | | | SSNTANQIKSLGISIKRIAGSNRYDTAARVAKAMGATSKAVILNGFLYADAPAVIP | |
| | | | YAAKNGYPILFTNKTSINSATTSVIKDKGISSTVVVGGTGSISNTVYNKLPSPTRI | |
| | | | SGSNRYELAANIVQKLNLSTSTVYVSNGFSYPDSIAGATLAAKKKQSLILTNGENL | |
| | | | STGARKIIGSKNMSNFMIIGNTPAVSTKVANQLKNPVVGETIFIDPGHGDQDSGAI | |
| | | | GNGLLEKEVNLDIAKRVNTKLNASGALPVLSRSNDTFYSLQERVNKAASAQADLFL | |
| | | | SIHANANDSSSPNGSETYYDTTYQAANSKRLAEQIQPKLAANLGTRDRGVKTAAFY | |
| | | | VIKYSKMPSVLVETAFITNASDASKLKQAVYKDKAAQAIHDGTVSYYR | |
| 43 | Bacillus | Protein | MKKQIITATTAVVLGSTLFAGAASAQSIKVKKGDTLWDLSRKYDTTISKIKSENHL | lytE |
| | subtilis | | RSDIIYVGQTLSINGKSTSSKSSSSSSSSSTYKVKSGDSLWKISKKYGMTINELKK | |
| | | | LNGLKSDLLRVGQVLKLKGSTSSSSSSSSKVSSSSTSTYKVKSGDSLSKIASKYGT | |
| | | | TVSKLKSLNGLKSDVIYVNQVLKVKGTSTSSSKPASSSSSSSSKTSSTSLNVSKLV | |
| | | | SDAKALVGTPYKWGGTTTSGFDCSGFIWYVLNKQTSVGRTSTAGYWSSMKSIASPS | |
| | | | VGDFVFFTTYKSGPSHMGIYIGNNSFIHAGSDGVQISSLNNSYWKPRYLGAKRF | |
| 44 | Bacillus | Protein | MSVFTNSYIPVNKYTRPGLKLQGVKKCVLHYTANPGAGADNHRRYFSNAQVYASAH | blyA |
| | subtilis | | IFVDKAEAICIIPLNEVAYHANDIQQRDSAGNPYRGVAALKPNANFLSIGVEMCLE | |
| | | | KDGSFHSDTVERTEDVFVELCNKFGLDPIDDIVRHYDITHKNCPAPWVSNSQKFVD | |
| | | | FKNRVKAKMSGKSVSKASPTKPTTSSPSSSSAVSGSLKSKVDGLRFYSKPSWEDKD | |
| | | | VVGTVNKGIGFPTVVEKVKVGSAYQYKVKNSKGTTYYITASDKYVDVTGSVKTSSS | |
| | | | APKTTSTSSSSSSIKSVGKIKIVGVSSAAIVMDKPDRNSSKNIGTVKLGSTISISG | |
| | | | SVKGKNNSNGYWEVIYKGKRGYISGQFGSTI | |
| 45 | Bacillus | Protein | MKKFIALLFFILLLSGCGVNSQKSQGEDVSPDSNIETKEGTYVGLADTHTIEVTVD | bhlA |
| | subtilis | | NEPVSLDITEESTSDLDKFNSGDKVTITYEKNDEGQLLLKDIERAN | |
| 46 | Bacillus | Protein | MEMDITQYLSTQGPFAVLFCWLLFYVMKTSKERESKLYNQIDSQNEVLGKFSEKYD | bhlB |
| | subtilis | | VVIEKLDKIEQNEK | |
| 47 | Bacillus | Protein | MFENIDKGTIVRTLLLAIALLNQIMVMLGKAAFIINEEDINHLYDCLYTIFTIVFT | cwlA |
| | subtilis | | TSTTTAAWFKNNYITAKGKKQKQVLKKENLFK | |

### REFERENCES

Conrad, B., Savchenko, R. S., Breves, R., & Hofemeister, J. (1996). A T7 promoter-specific, inducible protein expression system for Bacillus subtilis. Molecular and General Genetics MGG, 250(2), 230-236.
Derré, I., Rapoport, G., Devine, K., Rose, M., & Msadek, T. (1999). ClpE, a novel type of HSP100 ATPase, is part of the CtsR heat shock regulon of Bacillus subtilis. Molecular microbiology, 32(3), 581-593.
González-Pastor, J. E. (2011). Cannibalism: a social behavior in sporulating Bacillus subtilis. FEMS microbiology reviews, 35(3), 415-424.
González-Pastor, J. E., Hobbs, E. C., & Losick, R. (2003). Cannibalism by sporulating bacteria. Science, 301(5632), 510-513.
Kovács, Á. T. (2019). Bacillus subtilis. Trends in microbiology, 27(8), 724-725.
Miethke, M., Hecker, M., & Gerth, U. (2006). Involvement of Bacillus subtilis ClpE in CtsR degradation and protein quality control. Journal of bacteriology, 188(13), 4610-4619.
Reuß, D. R., Schuldes, J., Daniel, R., & Altenbuchner, J. (2015). Complete genome sequence of Bacillus subtilis subsp. subtilis strain 3NA. Genome announcements, 3(2).
Wenzel, M., Müller, A., Siemann-Herzberg, M., & Altenbuchner, J. (2011). Self-inducible Bacillus subtilis expression system for reliable and inexpensive protein production by high-cell-density fermentation. Applied and environmental microbiology, 77(18), 6419-6425.
Warth L, Altenbuchner J. 2013. A new site-specific recombinase-mediated system for targeted multiple genomic deletions employing chimeric loxP and mrpS sites. Appl Microbiol Biotechnol 97:6845-6856. doi:10.1007/s00253-013-4827-8.
Phan et al. Novel plasmid-based expression vectors for intra- and extracellular production of recombinant proteins in Bacillus subtilis Protein Expr Purif: 2006, 46(2); 189-95
Vagner et al. 1998; Microbiology, 144(Pt11):3097-3104
Pérez Morales T G, Ho T D, Liu W T, Dorrestein P C, Ellermeier C D. Production of the cannibalism toxin SDP is a multistep process that requires SdpA and SdpB. J Bacteriol. 2013 July; 195(14):3244-51. doi: 10.1128/JB.00407-13. Epub 2013 May 17. PMID: 23687264; PMCID: PMC3697648.

1. An engineered host cell, wherein the host cell comprises:
a. modification of at least one nucleic acid sequence encoding a sporulation-promoting polypeptide, a cell lysis inhibitor polypeptide or a combination thereof; and optionally,
b. a first polynucleotide sequence encoding at least one cell lysis promoting polypeptide.

2. (canceled)

3. The engineered host cell of claim 1, wherein the host cell is derived from a Bacillus subtilis 168, 3NA, BMV9, or IIG-Bs-20-1 strain.

4. The engineered host cell of claim 1, wherein the at least one nucleic acid sequence encoding the sporulation-promoting polypeptide, or the cell lysis inhibitor polypeptide is selected from the polynucleotide sequence of sdpR, sdpI, spo0A, sigW, yfhL, yknW, yknX, yknY, yknZ, clpP, arbB or any combination thereof, and wherein the cell lysis promoting polypeptide is selected from SkfA, SkfB, SkfC, SkfE, SkfF, SkfG, SkfH, SdpA, SdpB, SdpC, SdpI, SdpR, LytA, LytB, LytC, LytE, BlyA, BhlA, BhlB, CwlA, or a derivative or fragment thereof.

5. (canceled)

6. The engineered host cell of claim 1, wherein the first polynucleotide sequence encoding the cell lysis promoting polypeptide comprises one or more of a nucleic acid sequence having at least 85%, 90%, 95% or 99% sequence identity with a sequence as provided in SEQ ID NOs: 1-12, 32-39 or a fragment or functional derivative thereof.

7. (canceled)

8. The engineered host cell of claim 1, wherein the first polynucleotide sequence comprises the nucleic acid sequence encoding a polypeptide having at least 85%, 90%, 95% or 99% sequence identity with SEQ ID NOs: 13-24, 40-47 or a fragment thereof.

9. (canceled)

10. The engineered host cell of claim 1, wherein the first polynucleotide sequence of (b) is operably linked to a first promoter, wherein the first promoter comprises a constitutive or an inducible promoter, and wherein the inducible promoter comprises a thermosensitive, a chemosensitive, or a photosensitive promoter.

11. (canceled)

12. (canceled)

13. The engineered host cell of claim 1, further comprising a second polynucleotide sequence comprising a second nucleic acid sequence encoding a heterologous polypeptide operably linked to a second promoter.

14. The engineered host cell of claim 13, wherein the heterologous polypeptide comprises a nutritive, therapeutic, enzymatic, and/or a food preservative protein.

15. The engineered host cell of claim 14, wherein the heterologous polypeptide is selected from casein, a casein subunit selected from α-casein, β-casein, or κ-casein or a variant, or derivative thereof, a heme protein selected from a hemoglobin, a soy leghemoglobin, or a myoglobulin or a variant or derivative thereof, a lactalbumin, a lactoglobulin, an ovalbumin, an ovotransferrin, an ovomucoid, an ovomucin, and a lysozyme.

16. (canceled)

17. (canceled)

18. (canceled)

19. The engineered host cell of claim 13, wherein the second promoter comprises a constitutive or an inducible promoter system, wherein the inducible promoter is a thermosensitive, a chemosensitive promoter.

20. (canceled)

21. The engineered host cell of claim 20, wherein the chemosensitive promoter is selected from P_{grac}100, P_{spac}, P_{xylA}, P_{licB}, PT7, PT7_{lac}, P_{manP}, and P_{manR}.

22. The engineered host cell of claim 19, wherein the constitutive promoter comprises a Pveg promoter.

23. The engineered host cell of claim 1, further comprising an expression vector, a plasmid vector, a bacteriophage, a transposon, or genomic DNA comprising the first polynucleotide sequence and/or the second polynucleotide sequence.

24. The engineered host cell of claim 23, comprising a plasmid vector wherein the plasmid vector is a dual expression vector comprising the first polynucleotide sequence and the second polynucleotide sequence.

25. A method of producing a heterologous polypeptide, the method comprising:
a. culturing an engineered host cell, wherein the engineered host cell comprises:
i. modification of at least one nucleic acid sequence encoding a sporulation-promoting polypeptide, a cell lysis inhibitor polypeptide or a combination thereof;
ii. an optional first polynucleotide sequence encoding at least one cell lysis promoting polypeptide operably linked to a first promoter; and
iii. a second polynucleotide sequence comprising a second nucleic acid sequence encoding a heterologous polypeptide operably linked to a second promoter;
b. expressing the heterologous polypeptide in the host cell;
c. inducing cell lysis by maintaining the host cell for a time and under conditions sufficient for expression of at least one cell lysis promoting polypeptide;
d. harvesting the heterologous polypeptide from culture supernatant.

26. (canceled)

27. (canceled)

28. (canceled)

29. (canceled)

30. (canceled)

31. (canceled)

32. The method of claim 25, wherein the heterologous polypeptide is selected from casein, a casein subunit selected from α-casein, β-casein, or κ-casein, a heme protein selected from a hemoglobin, a soy leghemoglobin, or a myoglobulin, a lactalbumin, a lactoglobulin, an ovalbumin, an ovotransferrin, an ovomucoid, an ovomucin, and a lysozyme.

33. (canceled)

34. (canceled)

35. (canceled)

36. An engineered cell lysis system for production of a heterologous polypeptide, the system comprising an engineered host cell, wherein the engineered host cell comprises:
i. modification of at least one nucleic acid sequence encoding a sporulation-promoting polypeptide, a cell lysis inhibitor polypeptide or a combination thereof;
ii. an optional first polynucleotide sequence encoding at least one cell lysis promoting polypeptide operably linked to a first promoter; and
iii. a second polynucleotide sequence comprising a second nucleic acid sequence encoding a heterologous polypeptide operably linked to a second promoter.

37. (canceled)

38. (canceled)

39. (canceled)

40. (canceled)
